(19) United States
(12) Patent Application Publication
(10) Pub. No.: US 2006/0121455 A1
(43) Pub. Date: Jun. 8, 2006

(54) COP protein design tool

(75) Inventors: William A. Goddard, Pasadena, CA (US); Nagarajan Vaidehi, Arcadia, CA (US); Deqiang Zhang, San Diego, CA (US)

Correspondence Address: FISH & NEAVE IP GROUP;ROPES & GRAY LLP, ONE INTERNATIONAL PLACE, BOSTON, MA, 02110-2624, US

(73) Assignee: California Institute of Technology, Pasadena, CA (US)

(21) Appl. No.: 10/414,452

(22) Filed: Apr. 14, 2003

(51) Int. Cl. C12Q 1/68; C40B 40/08; G06F 19/00

(52) U.S. Cl. 435/6; 702/20

(57) ABSTRACT

The instant invention provides methods and implementing computer software for designing mutant proteins (or “Target Protein or TP”) that will preferentially bind one list of prespecified ligands (Active Ligands or AL) with respect to another list of ligands (The Inactive Ligands or IL).

### REFERENCE TO RELATED APPLICATION

This application claims the benefit of U.S. Provisional Application No. 60/372,074, filed on Apr. 12, 2002, the entire content of which is incorporated herein by reference.

### BACKGROUND OF THE INVENTION

Many proteins, such as enzymes and transcription factors, have a preferred list of natural substrates, cofactors (such as vitamins, metal ions, etc.), natural binding partners (including other macromolecules such as nucleic acids or protein, steroid, lipids, mono- and poly-saccharides, etc.). Under certain circumstances, it might be desirable to replace these natural substrates, co-factors, or binding partners with analogs that are not usually used by these proteins, thus changing the specificity and/or activity of these proteins. It may be even possible, through protein engineering, to create novel activity/specificity for these proteins and expand the functionality of these proteins.

Protein engineering is a powerful tool for modification of the structural catalytic and binding properties of natural proteins and for the de novo design of artificial proteins. Protein engineering relies on an efficient recognition mechanism for incorporating mutant amino acids in the desired protein sequences. This process has been very useful for designing new macromolecules with precise control of composition and architecture. However, a major limitation is that the mutagenesis is restricted to the 20 naturally occurring amino acids. Interestingly, this limitation can be overcome by protein engineering itself, by using the product of a particular protein engineering process—engineered Aminoacyl tRNA Synthetase (AARS).

Proteins are synthesized with precise control over nucleotide sequences encoding such proteins, leading to the vast range of specific structures and functional properties observed in nature. Even so the monomer pool for proteins is limited to the 20 natural amino acids. Increasing the monomer pool by incorporating new amino acid analogs would allow development of fascinating new bioderived polymers exhibiting novel but well-controlled architectures [1, 2]. The ability to incorporate amino acid analogs into proteins would greatly expand our ability to rationally and systematically manipulate the structures of proteins, both to probe protein function and create proteins with new properties. For example, the ability to synthesize large quantities of proteins containing heavy atoms would facilitate protein structure determination; incorporating selenium-substituted serine can facilitate crystallization processes in proteins [4]; and the ability to site specifically substitute fluorophores or photo-cleavable groups into proteins in living cells would provide powerful tools for studying protein structure and functions in vivo [3]. One might also be able to enhance the properties of proteins by providing building blocks with new functional groups, such as an amino acid containing a keto-group.

Incorporation of novel amino acids in macromolecules has been successful to an extent. Biosynthetic assimilation of non-canonical amino acids into proteins has been achieved largely by exploiting the capacity of the wild type synthesis apparatus to utilize analogs of naturally occurring amino acids (Budisa 1995, Eur. J. Biochem 230: 788-796; Deming 1997, J. Macromol. Sci. Pure Appl. Chem A34; 2143-2150; Duewel 1997, Biochemistry 36: 3404-3416; van Hest and Tirrell 1998, FEBS Lett 428(1-2): 68-70; Sharma et al., 2000, FEBS Lett 467(1): 37-40). Nevertheless, the number of amino acids shown conclusively to exhibit translational activity in vivo is small, and the chemical functionality that has been accessed by this method remains modest. In designing macromolecules with desired properties, this poses a limitation since such designs may require incorporation of complex analogs that differ significantly from the natural substrates in terms of both size and chemical properties and hence, are unable to circumvent the specificity of the synthetases. Thus, to further expand the range of non-natural amino acids that can be incorporated, there is a need to manipulate the activity of the aminoacyl tRNA synthetases (AARS).

The in vivo incorporation of amino acid analogs into proteins is controlled in large measure by aminoacyl-tRNA synthetases (AARS), the class of enzymes that safeguards the fidelity of amino acid incorporation into proteins. Specifically, protein translation from an mRNA template is carried out by ribosomes. During the translation process, each tRNA is matched with its amino acid long before it reaches the ribosome. The match is made by a collection of enzymes known as the aminoacyl-tRNA synthetases (AARS). These enzymes charge each tRNA with the proper amino acid, thus allowing each tRNA to make the proper translation from the genetic code of DNA (and the mRNA transcribed from the DNA) into the amino acid code of proteins.

Most cells make twenty different aminoacyl-tRNA synthetases, one for each type of amino acid. These twenty enzymes are each optimized for function with its own particular amino acid and the set of tRNA molecules appropriate to that amino acid. Aminoacyl-tRNA synthetases must perform their tasks with high accuracy. Many of these enzymes recognize their tRNA molecules using the anticodon. These enzymes make about one mistake in 10,000. For most amino acids, this level of accuracy is not too difficult to achieve, since most of the amino acids are quite different from one another.

It has been demonstrated that the wild-type translational apparatus can be used to incorporate some amino acid analogs into protein [5-11]. However, the number of amino acid analogs incorporated in proteins in vivo is small, and the functionalities of these analogs have been limited. To expand the range of amino acid analogs that can be incorporated in vivo, it is desirable to manipulate the activity of the AARS [12, 13]. There has been steady progress in developing the twenty-first AARS-suppressor tRNA pairs in vivo [14, 15]. The biggest success is the design of a novel orthogonal tRNA and tyrosyl-tRNA synthetase {TyrRS) [from Methanococcus jannaschii tyrosyl tRNA synthetase (hereafter denoted as M. jann-TyrRS)] that incorporates O-methyl L-tyrosine (OMe-Tyr) site-specifically into protein in response to an amber nonsense codon [16]. Such procedures have tremendous potential to expand the genetic codes in living cells, but the current combinatorial experiments, which considered, in theory, 5²⁰ mutation trials on five residues expected to be at the binding site of the tyrosine ligand, can become cumbersome. In addition, it is likely that not all of the possible 5²⁰ mutation trials will be synthesized and screened due to practical limitations. Thus, there is a need to develop a method of designing mutant proteins of desired property (preferentially bind certain ligands over other ligands).

### SUMMARY OF THE INVENTION

This invention, Clash Opportunity Progressive Design (COP) is a method that can computationally design a mutant protein that would preferentially bind an analog ligand over the natural ligand occurring in the wild type protein binding. The method has been applied to design a mutant tyrosyl-tRNA synthetase that would selectively bind O-Methyl-L-Tyrosine rather than any natural amino acids.

One aspect of the invention provides a method for designing a mutant of a polypeptide, said mutant preferentially binds an analog ligand (AL) over at least one inactive ligand (IL) of said polypeptide, comprising: (a) providing (i) an atomic coordinate model for said polypeptide, which model includes coordinates for at least the binding pocket residues for said IL; (ii) an IL rotamer of the most favorable solution conformation, and (iii) one or more AL rotamers, each more stable in solution than a pre-determined level of solution conformation energy; (b) docking said IL rotamer and each said AL rotamers into said binding pocket, wherein the backbone of said IL rotamer and said AL rotamers remain unchanged with respect to one another; (c) for each said AL rotamers docked into said binding pocket, identifying, in said binding pocket, varying residues which have less favorable interactions with said AL rotamer than with said IL rotamer; (d) identifying a subset of mutations for each of said varying residues identified in (c), wherein each of said mutations alone allows said polypeptide with fixed backbone to form a more favorable interaction with said AL rotamer than with said IL rotamer; (e) for each said AL rotamer, generating candidate combinatorial mutations including mutations in at least two of said varying residues, and selecting combinatorial mutations which form a more favorable interaction with said AL rotamer than with said IL rotamer; (f) for each said AL rotamer, selecting stable combinatorial mutations by optimizing each combinatorial mutations selected in (e) with no ligand in said binding pocket.

In one embodiment, the method further comprises: (g) for each stable combinatorial mutation selected in (f), identifying one or more specific combinatorial mutations that preferentially bind said AL rotamer over a pool of ILs structurally similar to said IL.

In one embodiment, any of the above methods further comprises: (h) generating each combinatorial mutation finally selected in (f) or (g) and testing in vivo and/or in vitro for selectively incorporating said AL into proteins over said IL, or said pools of ILs structurally similar to said IL.

The following embodiments, especially when directed to different features/elements of the invention, can be combined in all possible permutations when appropriate.

In one embodiment, said polypeptide is an Aminoacyl tRNA Synthetase (AARS), said IL is a natural ligand of said AARS, and said AL is an analog of said natural ligand. In a preferred embodiment, said natural ligand is one of the twenty amino acids usually found in natural proteins.

In one embodiment, said AARS is a phenoalanyl-tRNA Synthetase (PheRS) or a tyrosyl-tRNA Synthetase (TyrRS).

In one embodiment, said AARS is from a bacteria. In a preferred embodiment, said AARS is from a eukaryote.

In one embodiment, said AL is a derivative of at least one of the 20 natural amino acids, with one or more functional groups not present in natural amino acids. For example, said functional group can be selected from the group consisting of: bromo-, iodo-, ethynyl-, cyano-, azido-, aceytyl, aryl ketone, a photolabile group, a fluorescent group, and a heavy metal.

In one embodiment, said AL is a derivative of Phe or Tyr.

In one embodiment, said polypeptide is a G-Protein Coupled Receptor (GPCR) selected from: an adrenergic receptor (AR), an endothelial differential gene (EDG), an Olfactory Receptor (OR), or a Sweet Receptor (SR).

In one embodiment, said binding pocket residues comprise residues having at least one atom within a pre-defined distance from any atom of said AL or said IL. For example, said pre-defined distance may be 6 \acute{Å}.

In one embodiment, said AL rotamers are provided in (iii) by generating various candidate rotamers of said AL over a grid of dihedral angles, and calculating stability of all candidate rotamers.

In one embodiment, the stability of said one or more AL rotamers are calculated using quantum mechanics, or a suitable force field with molecular mechanics, or both.

In one embodiment, said IL rotamer or said AL rotamers is/are docked into said binding pocket based on a known three-dimensional complex structure of said IL and said polypeptide.

In one embodiment, said IL rotamer or said AL rotamers is/are docked into said binding pocket based on a docking algorithm.

In one embodiment, said docking algorithm is HIERDOCK.

In one embodiment, step (c) is effectuated by calculating and comparing non-bond energy contribution towards said AL rotamer and said IL rotamer for each binding pocket residues. In a preferred embodiment, said non-bond energy contribution is calculated using a force field. For example, said force field can be selected from: AMBER, AMBER94, AMBER/OPLS, OPLS, OPLS-AA, CHARMM, CHARMM22, Discover, ECEPP/2, GROMOS, MM2, MM3, MM4, MMFF, MMFFs, MMFF94, or UFF.

In one embodiment, said force field is a DREIDING force field.

In one embodiment, said DREIDING force field considers function forms for Coulomb, van der Waals, and hydrogen bond interactions.

In one embodiment, the dielectric constant for said Coulomb functional form is distance dependent or distance independent.

In one embodiment, the charge for either said binding pocket residues, or said AL/IL rotamer, or both can be varied in said Coulomb functional form.

In one embodiment, said charge includes charge from experiment, or charge based on a model selected from: QEq, Del Re, MPEOE, or Gasteiger/PEOE.

In one embodiment, the functional form of said van der Waals interaction uses a Leonard-Jones potential.

In one embodiment, said Leonard-Jones potential is a 6-12 or 6-10 Leonard-Jones potential.

In one embodiment, the functional form of said van der Waals interaction uses a Morse potential.

In one embodiment, the functional form of said hydrogen bond interaction uses a three-body form.

In one embodiment, the functional form of said hydrogen bond interaction uses a two-body form or a four-body form.

In one embodiment, said non-bond energy contribution is calculated using quantum mechanics (QM).

In one embodiment, in step (c), said AL rotamer docked into said binding pocket has less than a threshold level of clash with the backbone of said polypeptide.

In one embodiment, said threshold is 50%.

In one embodiment, said varying residues are clash residues.

In one embodiment, for each said varying residue, step (d) is effectuated by substituting the wild-type amino acid at said varying residue with all 19 natural amino acids, one at a time, and selecting all substitutions that favor binding to said AL rotamer over said IL rotamer.

In one embodiment, the side-chain conformation of each of the 19 substituted natural amino acids is generated over a grid of dihedral angles.

In one embodiment, the side chain conformation of each of the 19 substituted natural amino acids is generated from a backbone-dependent rotamer library.

In one embodiment, the wild-type amino acid at said varying residue is substituted with all 19 natural amino acids, one at a time, using SCWRL.

In one embodiment, the wild-type amino acid at said varying residue is substituted with all 19 natural amino acids, one at a time, using SCAP.

In one embodiment, the wild-type amino acid at said varying residue is substituted with all 19 natural amino acids, one at a time, using a side-chain modeling method based on branch-and-bound or dead-end-elimination algorithm.

In one embodiment, the method further comprises optimizing the side-chain of each of the 19 substituted natural amino acids after substitution but before selection.

In one embodiment, said optimization is carried out by energy minimization.

In one embodiment, said energy minimization utilizes a force field.

In one embodiment, said force field is a DREIDING force field.

In one embodiment, said optimization is carried out by Molecular Dynamics or by using Monte Carlo techniques.

In one embodiment, substitutions that favor binding to said AL rotamer are selected based on a score for each substitution, said score comprising a weighed sum of (I) the differential non-bond interaction energy of the substituted varying residue with said IL rotamer and said AL rotamer, and (II) the differential non-bond interaction energy of the substituted varying residue with the remaining residues of said polypeptide.

In one embodiment, said differential non-bond interaction energy of (I) and said differential non-bond interaction energy of (II) are independently calculated using a force field or quantum mechanics.

In one embodiment, said weighed sum comprises 75-100% of said differential non-bond interaction energy of (I).

In one embodiment, said score includes desolvation penalty for said AL rotamer and said IL rotamer.

In one embodiment, said desolvation penalty is calculated using a continuum implicit solvent model.

In one embodiment, said continuum implicit solvent model is a Surface Generalized Born (SGB) model, a Solvent Accessible Surface Area (SASA)/Analytical Volume Generalized Born (AVGB) model, or a Poisson-Boltzmann (PB) model.

In one embodiment, said desolvation penalty calculation further includes adding explicit solvent molecules.

In one embodiment, said varying residues are opportunity residues.

In one embodiment, said opportunity residues are identified after identification of clash residues.

In one embodiment, said opportunity residues stabilizes said AL rotamer or destabilizes said IL rotamer or both.

In one embodiment, said opportunity residues takes advantage of hydrogen bond donor or acceptor atoms that are different between said AL rotamer and said IL rotamer.

In one embodiment, said opportunity residues are identified by their proximity to a large void space in said binding pocket after identifying and mutating clash residues.

In one embodiment, for each said varying residue, step (d) is effectuated by substituting the wild-type amino acid at said varying residue with all 19 natural amino acids, one at a time, and selecting all substitutions that favor binding to said AL rotamer over said IL rotamer.

In one embodiment, in step (e), side-chains of each generated combinatorial mutation are optimized to generate optimal side-chain conformation for each combinatorial mutation.

In one embodiment, said optimization is carried out using a DREIDING force field with conjugate gradient minimization.

In one embodiment, after side-chain optimization, each of said combinatorial mutation is globally optimized both with said AL rotamer and with said IL rotamer.

In one embodiment, said global optimization both with said AL rotamer and with said IL rotamer is independently carried out using a force field, Molecular Dynamics, or Monte Carlo techniques.

In one embodiment, the backbone of each said combinatorial mutation is not fixed during global optimization.

In one embodiment, in step (e), combinatorial mutations which form a more favorable interaction with said AL rotamer than with said IL rotamer are selected based on the differential binding energy of said combinatorial mutations with said AL rotamer and said IL rotamer.

In one embodiment, said differential binding energy is calculated with a force field or quantum mechanics.

In one embodiment, said differential binding energy is calculated with a DREIDING force field with SGB solvation or AVGB salvation.

In one embodiment, said differential binding energy is calculated with Equation 2.

In one embodiment, the binding energies for said interactions in steps (c)-(e) are calculated using Potential of Mean Force (PMF), average dynamic free energy, Free Energy Perturbation (FEP), or methods based on thermodynamic cycles.

In one embodiment, in step (f), for each combinatorial mutations selected in (e), the varying residue side-chains are reselected from a backbone-dependent rotamer library, and each said combinatorial mutation is globally optimized with no ligand, using a force field or quantum mechanics with a continuum implicit solvent model.

In one embodiment, said global optimization include explicit water in said binding pocket.

In one embodiment, the method further includes calculating the differential binding energy for the globally optimized combinatorial mutation with said AL rotamer and said IL rotamer, using a force field or quantum mechanics with a continuum implicit solvent model.

In one embodiment, the global optimization and the differential binding energy are calculated using Equation 2.

Another aspect of the invention provides a peptide or polypeptide incorporating one or more amino acid analogs, which peptide or polypeptide was produced using a mutant AARS designed by any of the suitable method (or combinations thereof) described above.

This aspect of the invention contains all the combinations of the features of the methods described above.

Another aspect of the invention provides a method for conducting a biotechnology business comprising: (i) identifying one or more mutant forms of an AARS sequence, by the method of claim 4, said mutant preferentially binds to an amino acid analog of a natural amino acid substrate of said AARS; (ii) providing a translation system including: (a) a transcript, or means for generating a transcript, that encodes a peptide or polypeptide, (b) an mutant AARS having said identified mutant AARS sequence(s), and (c) said amino acid analog, under circumstances wherein said mutant AARS catalyzes incorporation of said amino acid analog in said peptide or polypeptide.

In one embodiment, the method further comprises the step of providing a packaged pharmaceutical including the peptide or polypeptide, and instructions and/or a label describing how to administer or use said peptide or polypeptide.

This aspect of the invention contains all the combinations of the features of the methods described above.

Another aspect of the invention provides a recombinant AARS protein generated by any of the above suitable methods, said AARS protein comprising an optimized protein sequence that incorporates an amino acid analog of a natural amino acid substrate of said AARS into a protein in vivo.

This aspect of the invention contains all the combinations of the features of the methods described above.

Another aspect of the invention provides a nucleic acid sequence encoding a recombinant AARS protein described above.

This aspect of the invention contains all the combinations of the features of the methods described above.

Another aspect of the invention provides an expression vector comprising the nucleic acid sequence described above.

This aspect of the invention contains all the combinations of the features of the methods described above.

Another aspect of the invention provides a host cell comprising the nucleic acid sequence described above.

This aspect of the invention contains all the combinations of the features of the methods described above.

Another aspect of the invention provides an apparatus for designing a mutant of a polypeptide, said mutant preferentially binds an analog ligand (AL) over at least one inactive ligand (IL) of said polypeptide, said apparatus comprising: (A) means for providing (i) an atomic coordinate model for said polypeptide, which model includes coordinates for at least the binding pocket residues for said IL; (ii) an IL rotamer of the most favorable solution conformation, and (iii) one or more AL rotamers, each more stable in solution than a pre-determined level of solution conformation energy; (B) means for docking said IL rotamer and each said AL rotamers into said binding pocket, wherein the backbone of said IL rotamer and said AL rotamers remain unchanged with respect to one another; (C) for each said AL rotamers docked into said binding pocket, means for identifying, in said binding pocket, varying residues which have less favorable interactions with said AL rotamer than with said IL rotamer; (D) means for identifying a subset of mutations for each of said varying residues identified in (C), wherein each of said mutations alone allows said polypeptide with fixed backbone to form a more favorable interaction with said AL rotamer than with said IL rotamer; (E) for each said AL rotamer, means for generating candidate combinatorial mutations including mutations in at least two of said varying residues, and selecting combinatorial mutations which form a more favorable interaction with said AL rotamer than with said IL rotamer; and, (F) for each said AL rotamer, means for selecting stable combinatorial mutations by optimizing each combinatorial mutations selected in (E) with no ligand in said binding pocket.

In one embodiment, said polypeptide is an Aminoacyl tRNA Synthetase (AARS), said IL is a natural ligand of said AARS, and said AL is an analog of said natural ligand.

This aspect of the invention contains all the combinations of the features of the methods described above.

Another aspect of the invention provides a computer system for designing a mutant of a polypeptide, said mutant preferentially binds an analog ligand (AL) over at least one inactive ligand (IL) of said polypeptide, said computer system comprising computer instructions for: (a) providing (i) an atomic coordinate model for said polypeptide, which model includes coordinates for at least the binding pocket residues for said IL; (ii) an IL rotamer of the most favorable solution conformation, and (iii) one or more AL rotamers, each more stable in solution than a pre-determined level of solution conformation energy; (b) docking said IL rotamer and each said AL rotamers into said binding pocket, wherein the backbone of said IL rotamer and said AL rotamers remain unchanged with respect to one another; (c) for each said AL rotamers docked into said binding pocket, identifying, in said binding pocket, varying residues which have less favorable interactions with said AL rotamer than with said IL rotamer; (d) identifying a subset of mutations for each of said varying residues identified in (c), wherein each of said mutations alone allows said polypeptide with fixed backbone to form a more favorable interaction with said AL rotamer than with said IL rotamer; (e) for each said AL rotamer, generating candidate combinatorial mutations including mutations in at least two of said varying residues, and selecting combinatorial mutations which form a more favorable interaction with said AL rotamer than with said IL rotamer; (f) for each said AL rotamer, selecting stable combinatorial mutations by optimizing each combinatorial mutations selected in (e) with no ligand in said binding pocket.

In one embodiment, said polypeptide is an Aminoacyl tRNA Synthetase (AARS), said IL is a natural ligand of said AARS, and said AL is an analog of said natural ligand.

This aspect of the invention contains all the combinations of the features of the methods described above.

Another aspect of the invention provides a computer-readable medium storing a computer program executable by a plurality of server computers, the computer program comprising computer instructions for: (a) providing (i) an atomic coordinate model for said polypeptide, which model includes coordinates for at least the binding pocket residues for said IL; (ii) an IL rotamer of the most favorable solution conformation, and (iii) one or more AL rotamers, each more stable in solution than a pre-determined level of solution conformation energy; (b) docking said IL rotamer and each said AL rotamers into said binding pocket, wherein the backbone of said IL rotamer and said AL rotamers remain unchanged with respect to one another; (c) for each said AL rotamers docked into said binding pocket, identifying, in said binding pocket, varying residues which have less favorable interactions with said AL rotamer than with said IL rotamer; (d) identifying a subset of mutations for each of said varying residues identified in (c), wherein each of said mutations alone allows said polypeptide with fixed backbone to form a more favorable interaction with said AL rotamer than with said IL rotamer; (e) for each said AL rotamer, generating candidate combinatorial mutations including mutations in at least two of said varying residues, and selecting combinatorial mutations which form a more favorable interaction with said AL rotamer than with said IL rotamer; (f) for each said AL rotamer, selecting stable combinatorial mutations by optimizing each combinatorial mutations selected in (e) with no ligand in said binding pocket.

In one embodiment, said polypeptide is an Aminoacyl tRNA Synthetase (AARS), said IL is a natural ligand of said AARS, and said AL is an analog of said natural ligand.

This aspect of the invention contains all the combinations of the features of the methods described above.

Another aspect of the invention provides a computer data signal embodied in a carrier wave, comprising computer instructions for: (a) providing (i) an atomic coordinate model for said polypeptide, which model includes coordinates for at least the binding pocket residues for said IL; (ii) an IL rotamer of the most favorable solution conformation, and (iii) one or more AL rotamers, each more stable in solution than a pre-determined level of solution conformation energy; (b) docking said IL rotamer and each said AL rotamers into said binding pocket, wherein the backbone of said IL rotamer and said AL rotamers remain unchanged with respect to one another; (c) for each said AL rotamers docked into said binding pocket, identifying, in said binding pocket, varying residues which have less favorable interactions with said AL rotamer than with said IL rotamer; (d) identifying a subset of mutations for each of said varying residues identified in (c), wherein each of said mutations alone allows said polypeptide with fixed backbone to form a more favorable interaction with said AL rotamer than with said IL rotamer; (e) for each said AL rotamer, generating candidate combinatorial mutations including mutations in at least two of said varying residues, and selecting combinatorial mutations which form a more favorable interaction with said AL rotamer than with said IL rotamer; (f) for each said AL rotamer, selecting stable combinatorial mutations by optimizing each combinatorial mutations selected in (e) with no ligand in said binding pocket.

In one embodiment, said polypeptide is an Aminoacyl tRNA Synthetase (AARS), said IL is a natural ligand of said AARS, and said AL is an analog of said natural ligand.

This aspect of the invention contains all the combinations of the features of the methods described above.

Another aspect of the invention provides an apparatus comprising a computer readable storage medium having instructions stored thereon for: (i) accessing a datafile representative of the coordinates for a plurality of different rotamers of one or more amino acids and one or more analogs of said amino acids; (ii) accessing a datafile representative of a set of structure coordinates for amino acid residues that define a binding pocket for an aminoacyl tRNA synthetase; (iii) a set of modeling routines for (a) calculating differential non-bond interaction energies between residues of the binding pocket with both the amino acids and the amino acid analogs; (b) Altering one or more amino acid residues in the binding pocket for the aminoacyl tRNA synthetase (AARS) to produce one or more sets of Altered AARS structure coordinates defining Altered binding pockets that differentially favor binding of the amino avid analogs over the amino acids; (c) generating a list representative of optimized AARS sequences having preferential interactions with said amino acid analogs over said amino acids.

This aspect of the invention contains all the combinations of the features of the methods described above.

Another aspect of the invention provides a method for synthesizing a peptide or protein incorporating one or more amino acid analogs, comprising providing a translational system including: (i) a transcript, or means for generating a transcript, that encodes a peptide or polypeptide, and (ii) one or more mutant AARS having a mutated binding pocket, each said mutant AARS catalyze preferential incorporation of an amino acid analog of the natural amino acid substrate of said AARS into said peptide or protein under the conditions of the translation system.

In one embodiment, said translation system is a whole cell that expresses said one or more AARS.

In one embodiment, said translation system is a cell lysate or reconstituted protein preparation that is translation competent.

In one embodiment, at least one of said mutant AARS catalyzes incorporation of said amino acid analog with a K_{cat} at least 10 fold greater than the wild-type AARS.

In one embodiment, at least one of said mutant AARS catalyzes incorporation of said amino acid analog with a K_{cat} at least 10 fold greater than the K_{cat} of the incorporation of natural amino acid substrate of the wild-type AARS.

In one embodiment, at least one of said mutant AARS catalyzes incorporation of said amino acid analog with a K_{cat} at least 2 fold greater than the K_{cat} of the incorporation of natural amino acid substrate of the wild-type AARS.

In one embodiment, at least one of said mutant AARS has a sequence identified by any suitable methods described above.

This aspect of the invention contains all the combinations of the features of the methods described above.

### BRIEF DESCRIPTION OF THE DRAWINGS

FIG. 1. The flowchart of an illustrative embodiment of COP.

FIG. 2. The predicted binding site surrounding the OMe-Tyr in the COP designed mutant [Y32Q, D158A] M. jann-TyrRS. The mutated residues (Gln32 and Ala158) are labeled in blue. Ala67, Ala158, and Leu65 form a hydrophobic pocket for the methyl group. The amide N^{ε2} of Gln32 has close contact with the oxygen of OMe, while the O^{ε1} atom of Gln32 is stabilized by forming a weak hydrogen bond (3.58 \acute{Å}) with the main chain NH of Leu65 (Both may have intervening water molecules).

FIG. 3. The predicted structure for M. jann-TyrRS with explicit side chains for the Tyr32, Asp158 residues involved in the design. The Tyr ligand is Also shown (labeled in red). The predicted position of residues Glu107 and Leu62 (labeled in blue) M. jann-TyrRS.

FIG. 4. Panel 1: two rotamers of naph-Ala used in the design of Example 2 and Example 6; Panel 2: several non-natural amino acid analogs used in re-designing of mj-TyrRS; Panel 3: the two rotamers of keto-Phe used in Example 4; Panel 4: the two rotamers of NBD-Ala used in Example 5; Panel 5: the two rotamers of bpy-Ala used in Example 5.

FIG. 5. The ribbon representation of PheRS with Phe bound in the active site.

FIG. 6. Binding site of naph-Ala in the PheRS mutant designed by COP.

FIG. 7. A Graphic User Interface (GUI) for COP.

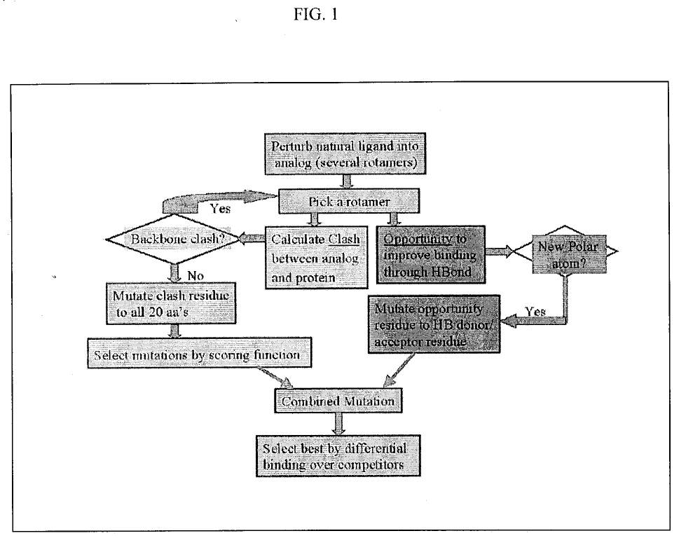

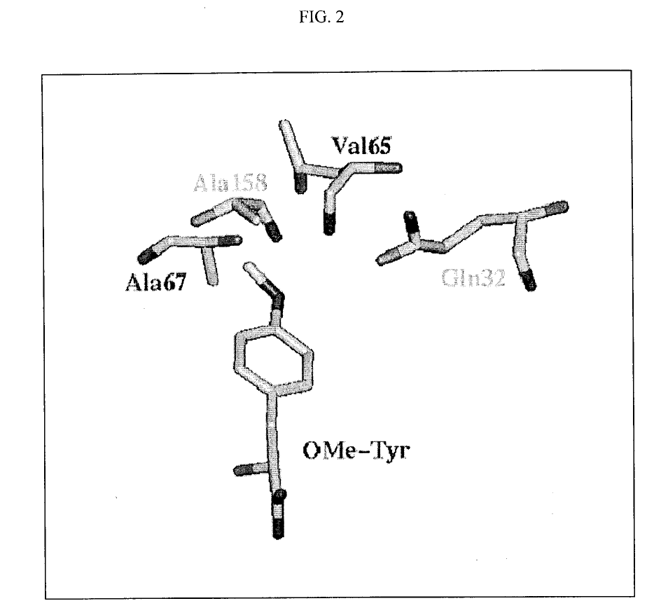

### DETAILED DESCRIPTION OF THE INVENTION

I. Overview

This invention, Clash Opportunity Progressive Design (COP), is a method that can computationally design a mutant protein that would preferentially bind an analog ligand over the natural ligand occurring in the wild type protein binding. It should be understood that the method can be used for any protein-ligand pair, Although in certain portions of the following description, the method has been described in detail from the view point of designing mutant AARS that would selectively bind an amino acid analog (such as O-Methyl-L-Tyrosine) over any natural amino acids. This, however, should not be construed to be limiting in any respect.

The Clash-Opportunity Progressive (COP) procedure can be used for structure based rational redesign of a binding site. To illustrate, one embodiment of the invention considers just a single Active Ligand (AL) that will bind specifically to the redesign protein or mutant (the Target Protein or TP), and just a single specific Inactive Ligand (IL), in this case, the natural ligand for the wild-type protein. Although other embodiments of the invention relates to redesigning a target protein for preferential binding to a list of ALs with respect to a list of ILs.

Generally, this procedure is useful in predicting which mutations in the binding site of the target protein are essential for preferential binding to a specific active ligand. For example, the design strategy can be used for designing AARS mutants that preferentially bind and activate a specific amino acid analog compared to all 20 natural amino acids (including the natural, preferred amino acid substrate of the AARS). The design goal is for the mutant protein to preferentially bind the target ligand (such as the amino acid analog) versus the wild type ligand (such as the wild-type amino acid or any other natural amino acid). This is achieved by calculating the differential binding energy of the desired AL (such as amino acid analog) against any other potential competitor inactive ligand (IL) that might bind selectively to the mutant. For example, in redesigning TyrRS, the differential binding energy of the amino acid analog against Tyr and Phe is calculated. If the analog is much larger than Tyr, then it may be necessary to consider Trp as a potential competitor for the redesigned mutant AARS.

COP is different from other protein design protocols, such as the one described in U.S. Pat. No. 6,269,312, in a number of ways. For example, COP designs for protein function to recognize a new ligand, such that the designed protein preferentially binds the new ligand over its natural ligand(s). In contrast, U.S. Pat. No. 6,269,312 aims at identifying the optimal side-chains for designing a protein with a certain desired fold (structure). Furthermore, COP calculates clashes between the protein and the ligand(s), and only designs the ligand binding site of the target protein. This allows COP to avoid lengthy calculations required in the protein core design.

The COP design procedure for designing mutant proteins generally comprises several interrelated steps as described below. Although these steps are named using numeric numbers (such as steps 1, 2, 3, etc.), the nomenclature is only used to roughly differentiate one step from the other. Although the COP method can be carried out in order from step 1 to step 7, it does not necessarily mean that step n must be carried out before step (n+1). In fact, a few steps can be combined, carried out in parallel, or in reverse order; while some steps may be carried out either before or after certain other steps. The boundaries between the steps are not necessarily clear cut, since certain calculations may belong to either of the two neighboring steps, or both.

Step 1. Conformation Determination. Step 1 of the COP design tool begins by obtaining accurate descriptions for the structures of the target protein, the inactive ligand (such as the natural amino acid substrate for a particular AARS), and the active ligand (such as the intended amino acid analog for the AARS). The protein structures can generally be obtained from known crystallographic or NMR data, if such data are readily available for the target protein. Alternatively, the structure description may be obtained using any of the modeling techniques described below.

In an exemplary embodiment, CHARMM22 charges with the nonpolar hydrogen charges summed onto the heavy atoms can be assigned to the a chain according to the parameters set forth in the DREIDING force field (Mayo et al., J. Phys. Chem. 94: 8897-8909, 1990). The protein can be neutralized by adding counterions (Na⁺ and Cl⁻) to the charged residues (Asp, Arg, Glu and Lys) and subject to a minimization of the potential energy by, for example, the conjugate gradient method using Surface Generalized Born continuum solvation method (Ghosh et al., J. Phys. Chem. B102: 10983-10990, 1998). The RMS in coordinates (CRMS) of all atoms after minimization may be obtained to verify if the CRMS values for the structure is well within experimental error. The crystal waters and other bound molecules can be removed for docking to maximize the searchable surface of the protein. The SGB continuum solvation method may be used for all structure optimizations and energy scoring with an internal protein dielectric constant of, for example, 2.5, for All calculations. Although other similar solvation models can be used.

The structure or favorable conformation of the ligands (IL or AL), including both the neutral and the zwitterion forms, can be obtained by generating various rotamers of the ligands over a grid of dihedral angles, and calculating their energies in solution using quantum mechanics (QM), or a suitable force field with molecular mechanics (MM), or both. An illustrative QM program is Jaguar (Schrodinger, Portland, Oreg.), and an illustrative MM program is Biograf (Accelrys, San Diego, Calif.). In certain embodiments, wherein a large number of possible ligand conformations in solution is possible, MM can be used in the initial calculation to identify the most promising few conformations, then QM can be used to more accurately determine the most favorable ligand conformations.

In an exemplary embodiment, The ligand conformations can be optimized in the extended conformation at the Hartree-Fock level of theory with a 6-31G** basis set, including Poisson-Boltzmann continuum dielectric solvation using the Jaguar computational suite (Tannor et al., J. Am. Chem. Soc. 116: 11875-11882, 1994) (Schrödinger, Portland, Oreg.). The Mulliken charges ascertained from this calculation can then be retained for the subsequent molecular mechanics simulations.

Hydrogen atoms, if not already present in the structures of the target protein and/or ligands, can be added manually, or using any of the suitable means, such as Biograf (Accelrys, San Diego, Calif.).

Step 2. Ligand Docking and Energy Minimization. Once the structures of the target protein, IL, and AL are obtained, the low energy rotamers of the AL can then be docked into the binding site of the target protein in Step 2.

In general, the binding site can be determined from crystal structure containing a ligand bound to the target protein. If no ligand is present in the protein structure, it can be modeled using various docking techniques. An example of such docking techniques is HierDock (U.S. Patent Application Publication No. US20020099506A1, or PCT publication WO0171347A1, incorporated herein by reference), which has been used extensively to predict and verify the binding of amino acids to AARS and other ligand-protein interactions, including binding of odorants to membrane-bound olfactory receptors (Floriano et al., P.N.A.S. U.S.A. 97: 10712-6, 2000), binding of outer membrane protein A to sugars (Datta et al., J. Am. Chem. Soc. 124: 5652-5653, 2002) (also see Datta et al., P.N.A.S. U.S.A. 99: 2636-41, 2002; and Datta et al., Proteins 50(2): 213-21, Feb. 1, 2003).

Briefly, the HierDock ligand screening protocol follows a hierarchical strategy for examining conformations, binding sites and binding energies. Such a hierarchical method has been shown to be necessary for docking algorithms (Halperin et al., Proteins: Struct. Funct. & Gene. 47, 409-443, 2002). The steps in HierDock involve using coarse-grain docking methods to generate several conformations of protein/ligand complexes followed by molecular dynamics (MD) simulations including continuum solvation methods performed on a subset of good conformations generated from the coarse-grain docking.

The three major steps in HierDock procedure in this paper are as follows:
First, a coarse-grain docking procedure to generate a set of conformations for ligand binding. For example, DOCK 4.0 (Ewing and Kuntz, J. Comput. Chem. 18: 1175-1189, 1997; Ewing et al., J. Comput. Aid. Mol. Design 15: 411-428, 2001, incorporated herein by reference) can be used to generate and score 20,000 configurations, of which 10% were ranked using the DOCK scoring function.
The 20 best conformations for each ligand from DOCK are selected, and subjected to annealing molecular dynamics (MD) to further optimize the conformation in the local binding pocket, allowing the atoms of the ligand to move in the field of the protein. In this step, the system may be heated and cooled from, for example, 50 K to 600 K, in steps of, for example, 10 K (0.05 ps at each temperature) for one cycle. At the end of annealing MD cycle, the best energy structure is retained. Annealing MD allows the ligand to readjust in the binding pocket to optimize its interaction with the protein. This fine grain optimization may be performed using MPSim (Lim et al., J. Comput. Chem. 18: 501-521, 1997) with DREIDING force field (supra) and continuum solvation methods, or any other similar methods. Surface Generalized Born (SGB) continuum solvent method can be used to obtain forces and energies resulting from the polarization of the solvent by the charges of the ligand and protein. This would allow calculation of the change in the ligand structure due to the solvent field and hence, obtain more realistic binding energies that take into account the solvation effects on the ligand/protein structure. The annealing MD procedure will generate 20 protein/ligand complexes for each ligand.
For the 20 structures generated by annealing MD simulations for each ligand, the potential energy of the full ligand/protein complex in aqueous solution can be minimized (using, for example, conjugate gradients minimization) using SGB. his step of protein/ligand-complex optimization is critical for obtaining energetically good conformations for the complex (cavity+ligand). Binding energies as the difference between the total energy of the ligand-protein complex in solvent (ΔG_{(protein+ligand)}), the sum of the total energies of the protein (ΔG₍ₚᵣₒₜₑᵢₙ₎), and the ligand separately in solvent (ΔG_{(ligand)}) can then be calculated. The energies of the protein and the ligand in solvent are calculated after independent energy minimization of the protein and the ligand separately in water. Solvation energies can be calculated using Poisson-Boltzmann continuum solvation method available in the software suite Delphi. The non-bond interaction energies can be calculated exactly using all pair interactions. Thus the binding energy is given by:
   ΔΔG_{calc}=ΔG_{(protein+ligand)}−ΔGₚᵣₒₜₑᵢₙ−ΔG_{ligand} (1)

Since the structure optimizations included solvation forces using the SGB continuum solvent approximation with the experimental dielectric constant, the calculated energies can be considered free energies (Hendsch and Tidor, Protein Sci. 8: 1381-92, 1999). This multi-step scanning procedure is based on docking via DOCK 4.0 coupled with fine-grain MM techniques. The coarse grain docked complex structures generated are scored with FF and differential salvation, which effectively filters the docked complexes to isolate the top contenders.

Based on the structure of the target protein—IL complex, the IL (such as the natural amino acid substrate) in the binding pocket of the target protein (such as the AARS of interest) can then be replaced with the energetically favorable rotamer(s) of the AL identified above (such as the intended amino acid analog), while keeping the backbone of the AL fixed, in order that the reaction center for the AL backbone remain unchanged with respect to the IL backbone. In the AARS scenario, this would ensure that the formation of the aminoacyl-AMP complex would be preserved for the intended analog.

Next, the potential energy of the TP-AL complex is minimized using any of the suitable methods, such as DREIDING force field in MPSim (Lim et al., J. Comput. Chem. 18: 501-521, 1997). Optionally, Poisson-Boltzmann dielectric continuum solvent was included to simulate solvation in water.

Step 3. Energy Calculation and Clash Identification. In Step 3, for each of the AL (analog) rotamer matched onto the binding site, the non-bond energy (Ek) contributions are calculated for each residue k in the binding pocket. The binding pocket residue is usually defined as target protein residues with any atom within a given distance (such as 6 \acute{Å}) from any atom of the AL (analog). These calculations can be carried out using any reliable force fields or quantum mechanics. One illustrative DREIDING force field (Mayo et al., J. Phys. Chem. 1990, 94:8897) method is described below in Equation 1, which considers the functional forms for such non-bond interaction energies as Coulomb, van der Waals, and hydrogen bond.

$${E}_{k} = {\sum }_{i , j} ( \frac{{q}_{i} {q}_{j}}{4 πɛ {r}_{ij}} + {D}_{e} ( {( \frac{{r}_{m}}{{r}_{ij}} )}^{12} - 2 {( \frac{{r}_{m}}{{r}_{ij}} )}^{6} ) + {D}_{HB} ( 5 {( \frac{{r}_{HB}}{{r}_{ij}} )}^{12} - 6 {( \frac{{r}_{HB}}{{r}_{ij}} )}^{10} ) {cos}^{4} θ ) \qquad ( Eq . 1 )$$

where i and j sum over all atoms in the ligand and protein residue k of interest, qᵢ and qⱼ are partial charges on atoms i and j, respectively. rᵢⱼ is the distance between atoms i and j, and rₘ and Dₑ are van der Waals distance and well depth of atoms i and j, r_{HB} and D_{HB} are hydrogen bond distance and well depth, respectively. θ is the hydrogen bond angle between atoms i, j and their bridging hydrogen atom. Please note that the hydrogen bond term is only evaluated for hydrogen bond pair atoms. When there is no bridging hydrogen atom for i and j, the hydrogen bond term is turned off by setting the value of θ as 90°.

This equation may be generally used to calculate non-bond interaction energy between a first and a second molecule. Generally, atom i (or j) represents an atom on the first molecule (such as an atom of the AL or IL), while atom j (or i) represents an atom on the second molecule (such as an atom belonging to the binding pocket residue k). However, this equation may also be used to calculate the non-bond interaction energy of a particular target protein residue (such as a mutated one, see the clash-relieving step below) with the remaining residues of the target protein. In that case, atom i may represent an atom of said particular (mutant) residue, while atom j represents an atom belonging to any one of the remaining residues of the target protein.

The same ligand docking and energy calculation can be done for each (or at least the most energetically favorable) IL rotamer.

Based on these calculations, the difference in interaction energy with the AL (analog) and the IL (wild-type amino acid) is calculated for each binding pocket residue. Those residues that have an unfavorable binding contribution to the AL (analog) will show positive differential interaction energies (DIE). Generally, these target protein residues either clash with the AL (analog), or do not make enough interactions with the AL (analog), thus representing the opportunity for improving target protein binding to the AL (analog). The following describes how these “clash residues” or “opportunity residues” can be identified in COP.

Clash-residue Identification and Clash Relief (Step 3a): Since the target protein backbone is fixed in Step 3a, it is required that any AL (analog) rotamer should not have a severe clash with the backbone of the target protein. Thus, any TP-AL complex in which AL (analog) rotamers have a severe clash with the target protein backbone are discarded without further consideration. “Severe clash” usually includes situations wherein more than a threshold value (say, 50%) of the total non-bond interaction energy between the ligand and residue k is contributed by interaction of the ligand atoms with the backbone atoms of residue k. In other words, in this particular example, less than 50% of the non-favorable interaction energy is contributed by interaction of the ligand atoms with the side-chain atoms of residue k. The percentage of contribution from backbone vs. side-chain atoms will be apparent from the calculation described above. The arbitrary “50%” cutoff value may be varied in either direction based on user preference or specific cases at hand.

Meanwhile, for each remaining AL (analog) rotamer, each binding pocket residue with minor (below threshold) backbone clash and/or with side-chain clash (collectively called “clash residues”) with the rotamer will be substituted by (or “mutated into”) the remaining 19 natural amino acids, one at a time, in order to find substitutions (or “mutations”) that can either relieve the clash or improve the differential interaction with the AL (analog) with respect to the IL (natural amino acid substrate of AARS), or both.

These “point mutations” of this clash-relieving Step 3a utilize rotameric conformations that can be generated over a grid of dihedral angles or by using side chain rotamer libraries (Bower et al., J. Mol. Biol. 267: 1268-82, 1997) (such as a backbone-dependent rotamer library), while the backbone of the target protein is held fixed during the process. After each single point mutation, the side chain alone may be optimized while the rest of the protein is kept fixed. This optimization step can be done using energy minimization (such as the one used in our example), Molecular Dynamics, or Monte Carlo techniques. The optimization step may be beneficial because the initial side-chain placement may not be optimum, and may lead to bad local contacts that could give misleading indications regarding this specific side-chain rotameric conformation.

Then the contribution from this mutated side-chain to the binding energy of both the AL (analog) and the IL (wild type amino acid) can be calculated, using, for example, Equation 1. Clashes of this mutated residue with neighboring residues in the target protein (“constraint energy”) are also calculated. A score will be generated for each mutation using a scoring energy function, which includes a weighted sum of the differential non-bond interaction energy of the mutated residue with the IL and the AL (analog), and the non-bond interaction energy of the mutated residue with the remaining residues of the target protein. To illustrate, weights of 0.75 to 1.0 can generally be used for ligand-protein interaction, and 0.0 to 0.25 can generally be used for protein residue—protein residue interactions. However, under certain circumstances, weights outside the range described above may Also be used. For each clash residue associated with a given rotamer, the differential binding energies for all twenty possible amino acids (including 19 substitutions) are calculated, and the best mutations are selected based on these scores (lower score is better). If none of the other 19 residues has a better score, then this particular residue position is either not changed, or changed to the few residues with scores within a predetermined cutoff value (such as within 5 kcal/mole of the best mutation). This precautionary selection may be beneficial since combined mutations (see below) involving these less-than-optimal residue changes may have better selectivity for the AL (analog), or other unexpected overall benefits.

Since the energy cost of desolvating a ligand (an amino acid or an analog) to place it in the binding pocket of a target protein can be important in some systems, desolvation penalty may also be added to the above energy calculation for the mutated residue. The desolvation penalty can be calculated using any of a variety of methods, such as SGB (Ghosh, J. Phys. Chem. B 102: 10983-10990, 1998), AVGB (Zamanakos, Ph.D. Thesis, Calif. Institute of Technology, Pasadena, Calif., 2001), or Poison-Boltzmann (Tannor et al., J. Am. Chem. Soc. 116: 11875-11882, 1994) solvation method.

For each AL (analog) rotamer with non-severe backbone clash and/or side-chain clash with the target protein, this procedure is repeated for all the “clash residues” (binding pocket residues showing a clash with the AL (analog) rotamer). This differential energy score between the AL (analog) and the IL (natural ligand) selects one or more candidate amino acid mutations for each of the clash residues for further consideration. Since it is the differential energy scores that are considered, both mutations that favor AL (analog) binding and ones that disfavor IL (the natural ligand) binding are considered. In summary, based on the differential energy scores, a subset of candidate amino acid changes for each clash residue is selected for later use in simultaneous combinatorial mutations.

Opportunity Residue Identification and Stabilizing Opportunity Mutations (Step 3b): After identifying candidate mutations for relieving clashes at the clash residues, COP then looks for “opportunity mutations” in the binding pocket that would stabilize the analog ligand or disrupt the bonding with the natural ligand. To illustrate, residues positioned near the AL (for example, within a cutoff value of 6 \acute{Å}) are examined for their ability to take advantage of, for example, hydrogen bond donor or acceptor atoms that are different between the AL (analog) and the IL (natural ligand). Alternatively, the void space in the binding pocket after making the clash mutations is calculated, and any residue that is close to a large void is considered as a candidate for a stabilizing point mutation.

These opportunity mutations are selected using the same procedure outlined above for the clash mutations. Briefly, either the IL (natural ligand) structure or the superimposed AL (analog) structure is fit into the active binding site of the wild-type target protein structure. COP then calculates the pair-wise non-bond interaction energies between the ligands (AL or IL) and the target protein residues in the binding site, using any of the suitable method (such as Equation 1) as described above in Step 3. Those residues that could possibly stabilize the AL (analog) and/or destabilize the IL (natural ligand) binding are chosen to be mutated. The selection may be similarly based on a pre-determined threshold differential energy/score, such as 5, 3, 2, 1, or 0.5 kcal/mole, so that any mutation that differentially favors AL binding is selected as a candidate opportunity mutation. “Differentially favor” means the mutated residue favors AL binding and disfavors IL binding, such that the differential energy/score is more negative (favored) than the threshold value. For each opportunity clash residue, the pre-set threshold value may be the same or different. After substituting all 20 possible amino acids in each opportunity position, based on the differential scores, a subset of amino acid changes for each opportunity residue is selected for later use in simultaneous combinatorial mutations.

Step 4. Combinatorial Mutations. The above steps (especially Steps 3a and 3b) lead to the identification of one or more clash residues and/or opportunity residues associated with a given rotamer, and a subset of candidate mutations for each of these clash or opportunity residues that are expected to either relieve clashes or provide stabilization opportunities to the binding of the AL (analog) in preference to the IL (wild type ligand). In Step 4 (simultaneous combinatorial mutation step), simultaneous mutation combines these chosen subsets of candidate mutations in all possible permutations. For example, if the clash analysis leads to 3 clash residues with 2, 3 and 4 candidate mutations, respectively, and the opportunity analysis leads to one opportunity residue with 5 candidate mutations (say, for making hydrogen bonds with the analog), then a total of 2×3×4×5=120 possible combinatorial mutants will be considered in this step. Each of these 120 mutations contains simultaneous mutation at all 4 residues. Alternatively, combinatorial mutations containing simultaneous mutations at less than all the identified clash and opportunity residues (less than 4 in the above hypothetical example) may be generated for further testing. For example, 3×4×5=60 combinatorial mutations may be generated, each involving only 3 (rather than 4) identified clash/opportunity residues.

Next, the best possible rotamer combination is generated for each of these combinatorial mutants by optimizing the side-chains, using, for example, conjugate gradient minimization or any other suitable means described below. In other words, residues near any of the mutated residues are re-examined to determine the optimal side-chain conformation. Having selected optimal side-chains for all combinatorial mutants, the structure of each combinatorial mutant protein as a whole is optimized, both with the IL (natural ligand) and with the AL (analog). This optimization can be done using energy minimization (such as the ones used in our examples), Molecular Dynamics, or Monte Carlo techniques. Finally, the differential binding energy (with respect to the AL/analog and the IL/natural amino acid) in each combinatorial mutant is calculated. Instead of a single IL, a pool of IL may be separately used in this calculation, and the result of each calculation (or at least the best competitor IL) compared to that of the AL (alternatively, this pool competition may be included as the last step, as described below in Step 6). These calculations can use any reliable force field or can use quantum mechanics. In one illustrative example below, Equation 2 with DREIDING Force Field and SGB solvation is used.
−ΔΔG=ΔG₍ₚᵣₒₜₑᵢₙ₎+ΔG_{(ligand)}−ΔG_{(protein+ligand)} (Equation 2)

Step 5. Relation of the free protein without the ligand. Step 4 above considers combinatorial mutations and side-chain conformations that enhance binding of the target protein to the AL (analog). However, it is possible that some combinatorial mutations would disrupt the folding of the free protein when the ligand is not present. Thus in Step 5, for the best combinatorial mutants selected above, the side-chains of each combinatorial mutant is re-optimized without any ligand in the binding site. This allows the side-chains of each combinatorial mutation to go into the binding site normally occupied by the ligand during the previous steps. Briefly, the side-chain conformations of each combinatorial mutants are first re-selected from a side-chain rotamer library, and the structure of the whole combinatorial mutant is optimized as above (for example, using Equation 2), including solvation (for example, using the SGB continuum solvent procedure). These calculations might include explicit water in the active site to better represent the stability of the active site without any ligand. Once the mutant structure is optimized without ligand, ligand is then fit within the binding site, and the potential energy of the resulting structure is minimized, using, for example, SGB solvation. This is done for both the AL (analog ligand) and the IL (natural ligand). This will generally lead to a weaker binding energy for the AL when compared to the step above, because the calculation now includes the penalty paid for pushing the side chains away from the binding site as the ligand binds. Although this “push-away” penalty applies to both the AL and the IL, the IL (natural ligand) may have a stronger overall binding energy. Thus the differential binding energy in this step will generally be smaller than in the step above. This differential binding energy here is denoted the “relaxed protein binding energy,” since the combinatorial mutants are optimized with no ligand in the binding pocket. The binding energy is defined the same as in Equation 2.

Step 6. Selection. If differential binding of the AL is only compared to a single IL to identify the best combinatorial mutant target protein above, it is possible that the identified combinatorial mutant, while preferring to bind the intended AL to the single IL, may nevertheless prefer to bind other IL(s) (such as those with similar structure as the single used IL) rather than the intended AL. For example, when redesigning a Tyrosyl-AARS for binding to a specific Tyrosine analog, it is important that the mutant Tyrosyl-AARS activates only the Tyrosine analog, but not any other natural amino acid such as Trp or Phe. At least, the redesigned AARS should preferentially bind the analog not only over the natural ligand Tyr, but also over Phe and Trp. Based on that consideration, if a single IL is used above, the best resulting candidate combinatorial mutants are tested further for binding to other structurally similar IL(s) (such as other natural amino acids). Thus, other likely IL (natural amino acid) competitors are separately docketed into both the relaxed and optimized binding sites, using the procedures described above. The binding energy is calculated for each ligand (AL vs. IL)/mutant pair. The mutants are finally ranked by the difference in binding energies between the AL (analog) and its IL competitors. The better binding energy is taken either from the relaxed or the optimized mutants. Only those combinatorial mutants that preferentially bind the AL over all potential IL competitors are selected.

In certain rare cases, it is possible that the designed mutant protein might not be able to fold correctly. For example, if there is charged residue placed in the protein core without favorable local stabilizing interactions, it is a strong destabilizing force. In order to detect such cases in post design, a consensus method may be used to evaluate the interactions for each residue involved in the design. The consensus is derived from all the AARS structures Applicants have been working one, including TyrRS (PDB: 2ts1, 3ts1, 4ts1), PheRS (PDB: 1b70), SerRS (PDB: 1ses, 1set, 1sry, 1fyf), ArgRS (PDB: 1bs2), MetRS (PDB: 1f41), HisRS (PDB: 1adj, 1hht). Table X lists the energies for each amino acid from the consensus:

Table X. Interactions energies of each amino acid in crystal structures of AARSs. These values are used to decide if an amino acid is in an unfavorable position.

| | Average Energy | Standard Deviation |
|---|---|---|
| Residue | (kcal/mol) | (kcal/mol) |
| Ala | −1.893 | 3.654 |
| Arg | −108.953 | 40.459 |
| Asn | −28.324 | 9.948 |
| Asp | −48.155 | 14.464 |
| Cys | −4.297 | 3.145 |
| Gln | −23.980 | 7.071 |
| Glu | −44.910 | 11.809 |
| Gly | −2.857 | 3.652 |
| His | −6.088 | 6.505 |
| Ile | 3.522 | 5.380 |
| Leu | 1.613 | 5.037 |
| Lys | −43.560 | 10.184 |
| Met | −2.067 | 4.624 |
| Phe | 6.536 | 6.326 |
| Pro | 8.183 | 6.653 |
| Ser | −6.136 | 5.444 |
| Thr | −6.364 | 5.733 |
| Trp | 16.568 | 7.371 |
| Tyr | 0.960 | 5.725 |
| Val | 1.578 | 4.564 |

In a case where a residue has lower interaction energy with its neighboring residues, a warning message will be given with a stability score of the residue. The score is defined by the energy difference divided by the standard deviation. A score higher than 2 generally means the residue is in a very unfavorable position, i.e., it is not making enough interactions with other residues to stabilize the fold.

Finally, Steps 1-6 are repeated for other low energy rotamers of the AL (analog). In the end, these calculations are expected to yield one or more combinatorial mutant target proteins that preferentially bind a specific rotamer of the AL (analog), for each (or at least one) of the all low energy rotamers of the AL (analog).

These general steps, including alternative embodiments are described in further detail in the sections which follow.

The instant invention has distinct advantageous over previous existing methods. Existing computational protein design methods focus mainly on design of protein core, i.e. the packing effect of various protein side chains combinatorially. Such methods generally use different algorithms to tackle the combinatorial nature of side chain packing, such as Dead End Elimination, Branch and Bound, Monte Carlo and Genetic algorithms, although some methods were extended to include surface residues. Nonetheless, these methods almost exclusively designs for a certain protein fold, i.e., replacing many residues to achieve better protein stability while maintaining the original fold. Since these methods are focused on design of a whole protein, computational efficiency required very crude energy evaluators. In addition, these existing design methods frequently suffer one or more of the following drawbacks:
Partial force field, which frequently fails to describe proteins accurately;
Incomplete energy function;
Empirical solvation, if present at all;
Fixed backbone (at all times).

On the other hand, the COP method makes the minimum number of rational structure-based mutations in a protein, so that the protein binds preferentially to the desired ligand compared to the designated inactive ligand (for example, the natural ligand). This preferential (or discriminatory) binding to a set of preferred analog ligand (AL) against a set of natural ligand or inactive ligand (IL) is a unique feature of the COP method, since it ensures at least a better (if not exclusive) interaction of the target protein with the analog over the inactive ligand. In the AARS case, it ensures the incorporation of the amino acid analogs with the intended structure into the final protein product. Without this build-in discriminatorial (preferential) binding calculation step, although the redesigned target protein may bind the intended AL, it may also bind the IL or other unintended ligands, sometimes with more affinity/specificity for other ligands than for the intended AL.

The COP method uses the principle minimum change design, which focuses on mutation of residues in the binding site, although there may be circumstances in which it is appropriate to modify residues outside the active site. This greatly simplifies the problem, partly because the number of residues involved is typically much less than the number of residues involved in a protein core design. In addition, the residues required to be changed (mutated) are often well separated, hence the probability of having combinatorial side-chain placement problem is much lower, if exists at all. Other advantages of the COP design methods are apparent in the discussion below about alternative embodiments. Briefly:
COP can use any force field valid for both protein and ligand (particularly valuable here are generic force fields such as DREIDING or UFF that are valid for a large part of the periodic table), and it can use quantum mechanics for the region of the active site.
COP uses a complete non-bond energy function such as in Equation 1. This function includes (Coulomb) electrostatic interactions (which may be described as point charges as in Eq. 1 or maybe described as distributed charges as in QEq (Rappe and Goddard, J. Phys. Chem. 95: 3358, 1991) or ReaxFF (van Duin et al., J. Phys. Chem. A 105: 9396-9409, 2001)), non-electrostatic non-bond interaction (referred to as van der Waals) which may be described by a 6-12 Leonard-Jones potential (or Morse form or exponential six), and an explicit hydrogen bond potential (which may be described by a radial potential (e.g. 10-12 Leonard-Jones) and may use a three-body cosine angle term as in Eq. 1.
Solvation is explicitly included in the COP design procedure. Continuum implicit solvation methods such as SGB or AVGB can be used to describe the role of solvation in the structure and energies of protein and ligand in water (or other) solvent. This greatly decreases the computation effort over the use of explicit solvent. However, explicit solvent molecules can be included in the evaluation of the best cases for final selection in the design.
The protein backbone can be allowed to move (distort in response to the mutations, solvent, and ligand) at any part of the algorithm except during clash identification. COP allows the protein backbone to be fully movable in any part of the optimization. The designed protein can be better optimized with backbone flexibility.
COP design adds the functionality of recognizing a new analog ligand to a mutant target protein (such as AARS), and it selects against any natural ligands. This ensures that the designed target protein (AARS) binds the analog (amino acid) exclusively. In the AARS example, the redesigned AARS can be used as an orthogonal tRBA-synthetase tRBA pair that corresponds to the 21^{st} amino acid (Wang & Schultz, Chem. Commun. 1-11, 2002).
   II. Definitions

The terms used in this specification (including the ones listed below) generally have their ordinary meanings in the art, within the context of this invention and in the specific context where each term is used. Certain terms are discussed below or elsewhere in the specification, to provide additional guidance to the practitioner in describing the compositions and methods of the invention and how to make and use them. Thus such discussion should not be construed to be limiting. The scope an meaning of any use of a term will be apparent from the specific context in which the term is used.

“About” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

“Amino acid analog” is meant to include all amino acid-like compounds that are similar in structure and/or overall shape to one or more of the twenty L-amino acids commonly found in naturally occurring proteins (Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y, as defined and listed in WIPO Standard ST.25 (1998), Appendix 2, Table 3). Amino acid analog can also be natural amino acids with modified side chains or backbones. Preferably, these analogs usually are not “substrates” for the AARSs because of the high specificity of the AARSs, although occasionally, certain analogs with structures/shapes sufficiently close to natural amino acids may be erroneously incorporated into the protein by AARSs, especially mutant AARSs with relaxed substrate specificity. In a preferred embodiment, the analogs share backbone structures, and/or even the most side chain structures of one or more natural amino acids, with the only difference(s) being containing one or more modified groups in the molecule. Such modification may include, without limitation, substitution of an atom (such as N) for a related atom (such as S), addition of a group (such as methyl, or hydroxyl group, etc.) or an atom (such as Cl or Br, etc.), deletion of a group (supra), substitution of a covalent bond (single bond for double bond, etc.), or combinations thereof. Amino acid analogs may include α-hydroxy acids, and β-amino acids, and can also be referred to as “modified amino acids,” “unnatural AARS substrates” or “non-canonical amino acids.”

The amino acid analogs may either be naturally occurring or non-naturally occurring; as will be appreciated by those in the art, any structure for which a set of rotamers is known or can be generated can be used as an amino acid analog. The side chains may be in either the (R) or the (S) configuration (or D- or L-configuration). In a preferred embodiment, the amino acids are in the (S) or L-configuration.

Preferably, the overall shape and size of the amino acid analogs are such that, upon being charged to tRNAs by the re-designed AARS, the analog-tRNA is a ribosomally accepted complex, that is, the tRNA-analog complex can be accepted by the prokaryotic or eukaryotic ribosomes in an in vivo or in vitro translation system.

“Anchor residues” are residue positions in AARS that maintain critical interactions between the AARS and the natural amino acid backbone.

“Backbone,” or “template” includes the backbone atoms and any fixed side chains (such as the anchor residue side chains) of the protein (e.g., AARS). For calculation purposes, the backbone of an analog is treated as part of the AARS backbone.

“Protein backbone structure” or grammatical equivalents herein is meant the three dimensional coordinates that define the three dimensional structure of a particular protein. The structures which comprise a protein backbone structure (of a naturally occurring protein) are the nitrogen, the carbonyl carbon, the α-carbon, and the carbonyl oxygen, along with the direction of the vector from the α-carbon to the β-carbon.

The protein backbone structure which is input into the computer can either include the coordinates for both the backbone and the amino acid side chains, or just the backbone, i.e. with the coordinates for the amino acid side chains removed. If the former is done, the side chain atoms of each amino acid of the protein structure may be “stripped” or removed from the structure of a protein, as is known in the art, leaving only the coordinates for the “backbone” atoms (the nitrogen, carbonyl carbon and oxygen, and the α-carbon, and the hydrogens attached to the nitrogen and α-carbon).

Optionally, the protein backbone structure may be altered prior to the analysis outlined below. In this embodiment, the representation of the starting protein backbone structure is reduced to a description of the spatial arrangement of its secondary structural elements. The relative positions of the secondary structural elements are defined by a set of parameters called supersecondary structure parameters. These parameters are assigned values that can be systematically or randomly varied to alter the arrangement of the secondary structure elements to introduce explicit backbone flexibility. The atomic coordinates of the backbone are then changed to reflect the altered supersecondary structural parameters, and these new coordinates are input into the system for use in the subsequent protein design automation. For details, see U.S. Pat. No. 6,269,312, the entire content incorporated herein by reference.

“Conformational energy” refers generally to the energy associated with a particular “conformation”, or three-dimensional structure, of a macromolecule, such as the energy associated with the conformation of a particular protein. Interactions that tend to stabilize a protein have energies that are represented as negative energy values, whereas interactions that destabilize a protein have positive energy values. Thus, the conformational energy for any stable protein is quantitatively represented by a negative conformational energy value. Generally, the conformational energy for a particular protein will be related to that protein's stability. In particular, molecules that have a lower (i.e., more negative) conformational energy are typically more stable, e.g., at higher temperatures (i.e., they have greater “thermal stability”). Accordingly, the conformational energy of a protein may also be referred to as the “stabilization energy.”

Typically, the conformational energy is calculated using an energy “force-field” that calculates or estimates the energy contribution from various interactions which depend upon the conformation of a molecule. The force-field is comprised of terms that include the conformational energy of the alpha-carbon backbone, side chain—backbone interactions, and side chain—side chain interactions. Typically, interactions with the backbone or side chain include terms for bond rotation, bond torsion, and bond length. The backbone-side chain and side chain-side chain interactions include van der Waals interactions, hydrogen-bonding, electrostatics and solvation terms. Electrostatic interactions may include Coulombic interactions, dipole interactions and quadrapole interactions). Other similar terms may also be included. Force-fields that may be used to determine the conformational energy for a polymer are well known in the art and include the CHARMM (see, Brooks et al., J. Comp. Chem. 4:187-217, 1983; MacKerell et al., in The Encyclopedia of Computational Chemistry, Vol. 1:271-277, John Wiley & Sons, Chichester, 1998), AMBER (see, Cornell et al., J. Amer. Chem. Soc. 1995, 117:5179; Woods et al., J. Phys. Chem. 1995, 99:3832-3846; Weiner et al., J. Comp. Chem. 1986, 7:230; and Weiner et al., J. Amer. Chem. Soc. 1984, 106:765) and DREIDING (Mayo et al., J. Phys. Chem. 1990, 94:8897) force-fields, to name but a few.

In a preferred implementation, the hydrogen bonding and electrostatics terms are as described in Dahiyat & Mayo, Science 1997 278:82). The force field can also be described to include atomic conformational terms (bond angles, bond lengths, torsions), as in other references. See e.g., Nielsen J E, Andersen K V, Honig B, Hooft R W. W, Klebe G, Vriend G, & Wade R C, “Improving macromolecular electrostatics calculations,” Protein Engineering, 12: 657662(1999); Stikoff D, Lockhart D J, Sharp K A & Honig B, “Calculation of electrostatic effects at the amino-terminus of an alpha-helix,” Biophys. J., 67: 2251-2260 (1994); Hendscb Z S, Tidor B, “Do salt bridges stabilize proteins—a continuum electrostatic analysis,” Protein Science, 3: 211-226 (1994); Schneider J P, Lear J D, DeGrado W F, “A designed buried salt bridge in a heterodimeric coil,” J. Am. Chem. Soc., 119: 5742-5743 (1997); Sidelar C V, Hendsch Z S, Tidor B, “Effects of salt bridges on protein structure and design,” Protein Science, 7: 1898-1914 (1998). Solvation terms could also be included. See e.g., Jackson S E, Moracci M, elMastry N, Johnson C M, Fersht A R, “Effect of Cavity-Creating Mutations in the Hydrophobic Core of Chymotrypsin Inhibitor 2,” Biochemistry, 32: 11259-11269 (1993); Eisenberg, D & McLachlan A D, “Solvation Energy in Protein Folding and Binding,” Nature, 319: 199-203 (1986); Street A G & Mayo S L, “Pair-wise Calculation of Protein Solvent-Accessible Surface Areas,” Folding & Design, 3: 253-258 (1998); Eisenberg D & Wesson L, “Atomic solvation parameters applied to molecular dynamics of proteins in solution,” Protein Science, 1: 227-235 (1992); Gordon & Mayo, supra.

“Coupled residues” are residues in a molecule that interact, through any mechanism. The interaction between the two residues is therefore referred to as a “coupling interaction.” Coupled residues generally contribute to polymer fitness through the coupling interaction. Typically, the coupling interaction is a physical or chemical interaction, such as an electrostatic interaction, a van der Waals interaction, a hydrogen bonding interaction, or a combination thereof. As a result of the coupling interaction, changing the identity of either residue will affect the “fitness” of the molecule, particularly if the change disrupts the coupling interaction between the two residues. Coupling interaction may also be described by a distance parameter between residues in a molecule. If the residues are within a certain cutoff distance, they are considered interacting.

“Fitness” is used to denote the level or degree to which a particular property or a particular combination of properties for a molecule, e.g., a protein, are optimized. In certain embodiments of the invention, the fitness of a protein is preferably determined by properties which: a user wishes to improve. Thus, for example, the fitness of a protein may refer to the protein's thermal stability, catalytic activity, binding affinity, solubility (e.g., in aqueous or organic solvent), and the like. Other examples of fitness properties include enantioselectivity, activity towards non-natural substrates, and alternative catalytic mechanisms. Coupling interactions can be modeled as a way of evaluating or predicting fitness (stability). Fitness can be determined or evaluated experimentally or theoretically, e.g. computationally.

Preferably, the fitness is quantitated so that each molecule, e.g., each amino acid will have a particular “fitness value”. For example, the fitness of a protein may be the rate at which the protein catalyzes a particular chemical reaction, or the protein's binding affinity for a ligand. In a particularly preferred embodiment, the fitness of a protein refers to the conformational energy of the polymer and is calculated, e.g., using any method known in the art. See, e.g. Brooks B. R., Bruccoleri R E, Olafson, B D, States D J, Swaminathan S & Karplus M, “CHARMM: A Program for Macromolecular Energy, Minimization, and Dynamics Calculations,” J. Comp. Chem., 4: 187-217 (1983); Mayo S L, Olafson B D & Goddard W A G, “DREIDING: A Generic Force Field for Molecular Simulations,” J. Phys. Chem., 94: 8897-8909 (1990); Pabo C 0 & Suchanek E G, “Computer-Aided Model-Building Strategies for Protein Design,” Biochemistry, 25: 5987-5991 (1986), Lazar G A, Desjarlais J R & Handel T M, “De Novo Design of the Hydrophobic Core of Ubiquitin,” Protein Science, 6: 1167-1178 (1997); Lee C & Levitt M, “Accurate Prediction of the Stability and Activity Effects of Site Directed Mutagenesis on a Protein Core,” Nature, 352: 448-451 (1991); Colombo G & Merz K M, “Stability and Activity of Mesophilic Subtilisin E and Its Thermophilic Homolog: Insights from Molecular Dynamics Simulations,” J. Am. Chem. Soc., 121: 6895-6903 (1999); Weiner S J, Kollman P A, Case D A, Singh U C, Ghio C, Alagona G, Profeta S J, Weiner P, “A new force field for molecular mechanical simulation of nucleic acids and proteins,” J. Am. Chem. Soc., 106: 765-784 (1984). Generally, the fitness of a protein is quantitated so that the fitness value increases as the property or combination of properties is optimized. For example, in embodiments where the thermal stability of a protein is to be optimized (conformational energy is preferably decreased), the fitness value may be the negative conformational energy; i.e., F=−E.

The “fitness “contribution” of a protein residue refers to the level or extent f(iₐ) to which the residue iₐ, having an identity a, contributes to the total fitness of the protein. Thus, for example, if changing or mutating a particular amino acid residue will greatly decrease the protein's fitness, that residue is said to have a high fitness contribution to the polymer. By contrast, typically some residues iₐ in a protein may have a variety of possible identities a without affecting the protein's fitness. Such residues, therefore have a low contribution to the protein fitness.

“Dead-end elimination” (DEE) is a deterministic search algorithm that seeks to systematically eliminate bad rotamers and combinations of rotamers until a single solution remains. For example, amino acid residues can be modeled as rotamers that interact with a fixed backbone. The theoretical basis for DEE provides that, if the DEE search converges, the solution is the global minimum energy conformation (GMEC) with no uncertainty (Desmet et al., 1992).

Dead end elimination is based on the following concept. Consider two rotamers, iᵣ and iₜ, at residue i, and the set of all other rotamer configurations {S} at all residues excluding i (of which rotamer jₛ is a member). If the pair-wise energy contributed between iᵣ and jₛ is higher than the pair-wise energy between iₜ and jₛ for all {S}, then rotamer iᵣ cannot exist in the global minimum energy conformation, and can be eliminated. This notion is expressed mathematically by the inequality.

$$E ( {i}_{r} ) + {\sum }_{j \neq i}^{N} E ( {i}_{r} , {j}_{s} ) > E ( {i}_{t} ) + {\sum }_{j \neq i}^{N} E ( {i}_{t} , {j}_{s} ) \{ S \} \qquad ( Equation A )$$

If this expression is true, the single rotamer iᵣ can be eliminated (Desmet et al., 1992).

In this form, Equation A is not computationally tractable because, to make an elimination, it is required that the entire sequence (rotamer) space be enumerated. To simplify the problem, bounds implied by Equation A can be utilized:

$$E ( {i}_{r} ) + {\sum }_{j \neq i}^{N} min ( s ) E ( {i}_{r} , {j}_{s} ) > E ( {i}_{t} ) + {\sum }_{j \neq i}^{N} max ( s ) E ( {i}_{t} , {j}_{s} ) \{ S \} \qquad ( Equation B )$$

Using an analogous argument, Equation B can be extended to the elimination of pairs of rotamers inconsistent with the GMEC. This is done by determining that a pair of rotamers iᵣ at residue i and jₛ at residue j, always contribute higher energies than rotamers iᵤ and jᵥ with all possible rotamer combinations {L}. Similar to Equation B, the strict bound of this statement is given by:

$$ɛ ( {i}_{r} , {j}_{s} ) + {\sum }_{k \neq i , j}^{N} min ( t ) ɛ ( {i}_{r} , {j}_{s} , {k}_{t} ) > ɛ ( {i}_{u} , {j}_{v} ) + {\sum }_{k \neq i , j}^{N} max ( t ) ɛ ( {i}_{u} , {j}_{v} , {k}_{i} ) \qquad ( Equation C )$$

where ε is the combined energies for rotamer pairs
ε(iᵣ,jₛ)=E(iᵣ)+E(jₛ)+E(iᵣ,jₛ (Equation D), and
ε(iᵣ,jₛ,kₜ)=E(iᵣ,kₜ)+E(jₛ,kₜ (Equation E).

This leads to the doubles elimination of the pair of rotamers iᵣ and jₛ, but does not eliminate the individual rotamers completely as either could exist independently in the GMEC. The doubles elimination step reduces the number of possible pairs (reduces S) that need to be evaluated in the right-hand side of Equation 6, allowing more rotamers to be individually eliminated.

The singles and doubles criteria presented by Desmet et al. fail to discover special conditions that lead to the determination of more dead-ending rotamers For instance, it is possible that the energy contribution of rotamer iₜ is always lower than iᵣ without the maximum of iₜ being below the minimum of iᵣ. To address this problem, Goldstein 1994 presented a modification of the criteria that determines if the energy profiles of two rotamers cross. If they do not, the higher energy rotamer can be determined to be dead-ending. The doubles calculation significantly more computational time than the singles calculation. To accelerate the process, other computational methods have been developed to predict the doubles calculations that will be the most productive (Gordon & Mayo, 1998). These kinds of modifications, collectively referred to as fast doubles, significantly improved the speed and effectiveness of DEE.

Several other modifications also enhance DEE. Rotamers from multiple residues can be combined into so-called super-rotamers to prompt further eliminations (Desmet et al., 1994; Goldstein, 1994). This has the advantage of eliminating multiple rotamers in a single step. In addition, it has been shown that “splitting” the conformational space between rotamers improves the efficiency of DEE (Pierce et al., 2000). Splitting handles the following special case. Consider rotamer iᵣ. If a rotamer iₜ₁ contributes a lower energy than iᵣ for a portion of the conformational space, and a rotamer iₜ₂ has a lower energy than iᵣ for the remaining fraction, then iᵣ can be eliminated. This case would not be detected by the less sensitive Desmet or Goldstein criteria. In the preferred implementations of the invention as described herein, all of the described enhancements to DEE were used.

For further discussion of these methods see, Goldstein, R. F. (1994), Efficient rotamer elimination applied to protein side-chains and related spin glasses, Biophysical Journal 66, 1335-1340; Desmet, J., De Maeyer, M., Hazes, B. & Lasters, I. (1992), The dead-end elimination theorem and its use in protein side-chain positioning. Nature 356,539-542; Desmet, J., De Macyer, M. & Lasters, I. (1994), In The Protein Folding Problem and Tertiary Structure Prediction (Jr., K. M. & Grand, S. L., eds.), pp. 307-337 (Birkhauser, Boston); De Maeyer, M., Desmet, J. & Lasters, I. (1997), all in one: a highly detailed rotamer library improves both accuracy and speed in the modeling of side chains by dead-end elimination, Folding & Design 2, 53-66, Gordon, D. B. & Mayo, S. L. (1998), Radical performance enhancements for combinatorial optimization algorithms based on the dead-end elimination theorem, Journal of Computational Chemistry 19, 1505-1514; Pierce, N. A., Spriet, J. A., Desmet, J., Mayo, S. L., (2000), Conformational splitting: A more powerful criterion for dead-end elimination; Journal of Computational Chemistry 21, 999-1009.

“Expression system” means a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors.

“Host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells may be cultured in vitro or one or more cells in a non-human animal (e.g., a transgenic animal or a transiently transfected animal).

The methods of the invention may include steps of comparing sequences to each other, including wild-type sequence to one or more mutants. Such comparisons typically comprise alignments of polymer sequences, e.g., using sequence alignment programs and/or algorithms that are well known in the art (for example, BLAST, FASTA and MEGALIGN, to name a few). The skilled artisan can readily appreciate that, in such alignments, where a mutation contains a residue insertion or deletion, the sequence alignment will introduce a “gap” (typically represented by a dash, “-”, or “Δ”) in the polymer sequence not containing the inserted or deleted residue.

“Homologous”, in all its grammatical forms and spelling variations, refers to the relationship between two proteins that possess a “common evolutionary origin”, including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

The term “sequence similarity”, in all its grammatical forms, refers to the degree of identity or correspondence between nucleic acid or amino acid sequences that may or may not share a common evolutionary origin (see, Reeck et al., supra). However, in common usage and in the instant application, the term “homologous”, when modified with an adverb such as “highly”, may refer to sequence similarity and may or may not relate to a common evolutionary origin.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., Molecular Cloning: A Laboratory Manual, Second Edition (1989) Cold Spring Harbor Laboratory Press, Cold Spring Harbor, N.Y.). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. SSC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

Unless specified, the term “standard hybridization conditions” refers to a Tₘ of about 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides lower melting temperature. Because the melting temperature of oligonucleotides will depend on the length of the oligonucleotide sequences involved, suitable hybridization temperatures will vary depending upon the oligonucleotide molecules used. Exemplary temperatures may be 37° C. (for 14-base oligonucleotides), 48° C. (for 17-base oligonucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

“Polypeptide,” “peptide” or “protein” are used interchangeably to describe a chain of amino acids that are linked together by chemical bonds called “peptide bonds.” A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

“Rotamer” is defined as a set of possible conformers for each amino acid or analog side chain. See Ponder, et al., Acad. Press Inc. (London) Ltd. pp. 775-791 (1987); Dunbrack, et al., Struc. Biol. 1(5):334-340 (1994); Desmet, et al., Nature 356:539-542 (1992). A “rotamer library” is a collection of a set of possible/allowable rotameric conformations for a given set of amino acids or analogs. There are two general types of rotamer libraries: “backbone dependent” and “backbone independent.” A backbone dependent rotamer library allows different rotamers depending on the position of the residue in the backbone; thus for example, certain leucine rotamers are allowed if the position is within an α helix, and different leucine rotamers are allowed if the position is not in an α-helix. A backbone independent rotamer library utilizes all rotamers of an amino acid at every position. In general, a backbone independent library is preferred in the consideration of core residues, since flexibility in the core is important. However, backbone independent libraries are computationally more expensive, and thus for surface and boundary positions, a backbone dependent library is preferred. However, either type of library can be used at any position.

“Variable residue position” herein is meant an amino acid position of the protein to be designed that is not fixed in the design method as a specific residue or rotamer, generally the wild-type residue or rotamer. It should be noted that even if a position is chosen as a variable position, it is possible that the methods of the invention will optimize the sequence in such a way as to select the wild type residue at the variable position. This generally occurs more frequently for core residues, and less regularly for surface residues. In addition, it is possible to fix residues as non-wild type amino acids as well.

“Fixed residue position” means that the residue identified in the three dimensional structure as being in a set conformation. In some embodiments, a fixed position is left in its original conformation (which may or may not correlate to a specific rotamer of the rotamer library being used). Alternatively, residues may be fixed as a non-wild type residue depending on design needs; for example, when known site-directed mutagenesis techniques have shown that a particular residue is desirable (for example, to eliminate a proteolytic site or alter the substrate specificity of an AARS), the residue may be fixed as a particular amino acid. Residues which can be fixed include, but are not limited to, structurally or biologically functional residues. For example, the anchor residues.

In certain embodiments, a fixed position may be “floated”; the amino acid or analog at that position is fixed, but different rotamers of that amino acid or analog are tested. In this embodiment, the variable residues may be at least one, or anywhere from 0.1% to 99.9% of the total number of residues. Thus, for example, it may be possible to change only a few (or one) residues, or most of the residues, with all possibilities in between.

III. Illustrative Embodiments of the COP Design Tool

A. Atomic Coordinates and Other Sequence/Structural Information for Proteins

An accurate description of the design molecules (such as the target protein) using the terms of atomic coordinates is important for the computational design approach of the instant invention. Generally speaking, the protein structure description should be in high quality, preferably obtained from either X-ray or NMR study. However, protein structures of high quality from theoretical modeling can also be used. In fact, in many cases, it is perfectly acceptable to use crystal structure of a homologous protein (for example, a homolog from a related species) or even a conserved domain to substitute the crystallographic structure description for the necessary atomic coordinates.

The crystal structures of thousands of proteins are currently available in the Brookhaven Protein Data Bank (PDB, see Bernstein et al., J. Mol. Biol. 112: 535-542, 1977). All contents of PDB are in the public domain. As of Mar. 11, 2003, PDB contains 20,317 total deposited structures, including 18289 protein/peptide/virus structures, 843 protein/nucleic acid complex structures, 1167 nucleic acid structures, and 18 carbohydrates. Presently, about 4000-4500 structures are being deposited to this database every year. More detailed information regarding all aspects of the PDB database can be found at the PDB website.

The structure database or Molecular Modeling DataBase (MMDB) contains experimental data from crystallographic and NMR structure determinations. The data for MMDB are obtained from the Protein Data Bank (PDB). The NCBI (National Center for Biotechnology Information) has cross-linked structural data to bibliographic information, to the sequence databases, and to the NCBI taxonomy. Cn3D, the NCBI 3D structure viewer, can be used for easy interactive visualization of molecular structures from Entrez.

The Entrez 3D Domains database contains protein domains from the NCBI Conserved Domain Database (CDD). Computational biologists define conserved domains based on recurring sequence patterns or motifs. CDD currently contains domains derived from two popular collections, Smart and Pfam, plus contributions from colleagues at NCBI, such as COG. The source databases also provide descriptions and links to citations. Since conserved domains correspond to compact structural units, CDs contain links to 3D-structure via Cn3D whenever possible.

To identify conserved domains in a protein sequence, the CD-Search service employs the reverse position-specific BLAST algorithm. The query sequence is compared to a position-specific score matrix prepared from the underlying conserved domain alignment. Hits may be displayed as a pair-wise alignment of the query sequence with a representative domain sequence, or as a multiple alignment. CD-Search now is run by default in parallel with protein BLAST searches. While the user waits for the BLAST queue to further process the request, the domain architecture of the query may already be studied. In addition, CDART, the Conserved Domain Architecture Retrieval Tool allows user to search for proteins with similar domain architectures. CDART uses precomputed CD-search results to quickly identify proteins with a set of domains similar to that of the query. For more details, see Marchler-Bauer et al., Nucleic Acids Research 31: 383-387, 2003; and Marchler-Bauer et al., Nucleic Acids Research 30: 281-283, 2002.

Particularly for AARS, as described above, most cells make twenty different aminoacyl-tRNA synthetases, one for each type of amino acid. The crystal structures of nearly all of these different enzymes are currently available in the Brookhaven Protein Data Bank (PDB, see Bernstein et al., J. Mol. Biol. 112: 535-542, 1977). A list of all the AARSs with solved crystal structures as of April 2001 is available on the PDB website. For example, the crystal structure of Thermus Aquaticus PhenylAlanyl tRNA Synthetase complexed with PhenylAlanine has a resolution of 2.7 \acute{Å}, and its PDB ID is 1B70.

In addition, a database of known aminoacyl tRNA synthetases has been published by Maciej Szymanski, Marzanna A. Deniziak and Jan Barciszewski, in Nucleic Acids Res. 29:288-290, 2001 (titled “Aminoacyl-tRNA synthetases database”). A corresponding website (http://rose.man.poznan.p1/aars/seq_main.html) provides details about all known AARSs from different species. For example, according to the database, the Isoleucyl-tRNA Synthetase for the radioresistant bacteria Deinococcus radiodurans (Accession No. AAF10907) has 1078 amino acids, and was published by White et al. in Science 286:1571-1577(1999); the Valyl-tRNA Synthetase for mouse (Mus musculus) has 1263 amino acids (Accession No. AAD26531), and was published by Snoek M. and van Vugt H. in Immunogenetics 49: 468-470(1999); and the PhenylAlanyl-tRNA Synthetase sequences for human, Drosophila, S. pombe, S. cerevisiae, Candida albicans, E. coli, and numerous other bacteria including Thermus aquaticus ssp. thermophilus are also available. The database was last updated in May 2000. Similar information for other newly identified AARSs can be obtained, for example, by conducting a BLAST search using any of the known sequences in the AARS database as query against the available public (such as the non-redundant database at NCBI, or “nr”) or proprietary private databases.

all these databases would provide atomic coordinates for proteins or other molecules with known structural information.

The AARSs may be from any organism, including prokaryotes and eukaryotes, with enzymes from bacteria, fungi, extremeophiles such as the archeobacteria, worm, insects, fish, amphibian, birds, animals (particularly mammals and particularly human) and plants all possible.

Alternatively, in certain embodiments, if the exact crystal structure of a particular protein/molecule is unknown, but its protein sequence is similar or homologous to a known protein sequence with a known crystal structure. In such instances, it is expected that the conformation of the protein in question will be similar to the known crystal structure of the homologous protein. The known structure may, therefore, be used as the structure for the protein of interest, or more preferably, may be used to predict the structure of the protein of interest (i.e., in “homology modeling” or “molecular modeling”). As a particular example, the Molecular Modeling Database (MMDB) described above (see, Wang et al., Nucl. Acids Res. 2000, 28:243-245; Marchler-Bauer et al., Nucl. Acids Res. 1999,27:240-243) provides search engines that may be used to identify proteins and/or nucleic acids that are similar or homologous to a protein sequence (referred to as “neighboring” sequences in the MMDB), including neighboring sequences whose three-dimensional structures are known. The database further provides links to the known structures along with alignment and visualization tools, such as Cn3D (developed by NCBI), RasMol, etc., whereby the homologous and parent sequences may be compared and a structure may be obtained for the parent sequence based on such sequence alignments and known structures.

The homologous protein sequence with known 3D-structure is preferably at least about 60%, or at least about 70%, or at least about 80%, or at least about 90%, or at least about 95% identical to the protein of interest, at least in the region that may be involved in interacting with another molecule of interest. Such potential binding sites may not be continuous in the primary amino acid sequence of the protein since distant amino acids may come together in the 3D-structure. In this case, sequence homology or identity can be calculated using, for example, the NCBI standard BLASTp programs for protein using default conditions, in regions aligned together (without insertions or deletions in either of the two sequences being compared) and including residues known to be involved in substrate amino acid binding. Alternatively, the homologous protein is preferably about 35%, or 40%, or 45%, or 50%, or 55% identical overall to the protein of interest. Many proteins with just about 20-25% overall sequence homology/identity turns out to be conserved in three-dimensional structure.

In the few cases where the structure for a particular protein sequence may not be known or available, it is typically possible to determine the structure using routine experimental techniques (for example, X-ray crystallography and Nuclear Magnetic Resonance (NMR) spectroscopy) and without undue experimentation. See, e.g., NMR of Macromolecules: A Practical Approach, G. C. K. Roberts, Ed., Oxford University Press Inc., New York (1993); Ishima R, Torchia D A, “Protein Dynamics from NMR,” Nat Struct Biol, 7: 740-743 (2000); Gardner K H, Kay L E, “The use of H-2, C-13, N-15 multidimensional NMR to study the structure and dynamics of proteins,” Annu. Rev. Bioph. Biom., 27: 357-406 (1998); Kay LE, “NMR methods for the study of protein structure and dynamics,” Biochem Cell Biol, 75: 1-15 (1997); Dayie K T, Wagner G, Lefevre J F, “Theory and practice of nuclear spin relaxation in proteins,” Annu Rev Phys Chem, 47: 243-282 (1996); Wuthrich K, “NMR—This and other methods for protein and nucleic-acid structure determination,” Acta Cyrstallogr. D, 51: 249-270 (1995); Kahn R, Carpentier P, Berthet-Colominas C, et al., “Feasibility and review of anomalous X-ray diffraction at long wavelengths in materials research and protein crystallography,” J. Synchrotron Radiat., 7: 131-138 (2000); Oakley A J, Wilce M C J, “Macromolecular crystallography as a tool for investigating drug, enzyme and receptor interactions,” Clin. Exp. Pharmacol. P., 27:145-151 (2000); Fourme R, Shepard W, Schiltz M, et al., “Better structures from better data through better methods: a review of developments in de novo macromolecular phasing techniques and associated instrumentation at LURE,” J. Synchrotron Radiat., 6: 834-844 (1999).

Alternatively, and in less preferable embodiments, the three-dimensional structure of a protein sequence may be calculated from the sequence itself and using ab initio molecular modeling techniques already known in the art. See e.g., Smith T F, LoConte L, Bienkowska J, et al., “Current limitations to protein threading approaches,” J. Comput. Biol., 4: 217-225 (1997); Eisenhaber F, Frommel C, Argos P, “Prediction of secondary structural content of proteins from their amino acid composition alone 2. The paradox with secondary structural class,” Proteins, 24: 169-179 (1996); Bohm G, “New approaches in molecular structure prediction,” Biophys Chem., 59: 1-32 (1996); Fetrow J S, Bryant S H, “New programs for protein tertiary structure prediction,” BioTechnol., 11: 479-484, (1993); Swindells M B, Thorton J M, “Structure prediction and modeling,” Curr. Opin. Biotech., 2: 512-519 (1991); Levitt M, Gerstein M, Huang E, et al., “Protein folding: The endgame,” Annu. Rev. Biochem., 66: 549-579 (1997). Eisenhaber F., Persson B., Argos P., “Protein-structure prediction—recognition of primary, secondary, and tertiary structural features from amino-acid-sequence,” Crit Rev Biochem Mol, 30:1-94(1995); Xia Y, Huang E S, Levitt M, et al., “Ab initio construction of protein tertiary structures using a hierarchical approach,” J. Mol. Biol., 300:171-185 (2000); Jones D T, “Protein structure prediction in the post genomicera,” Curr Opin Struc Biol, 10: 371-379 (2000). Three-dimensional structures obtained from ab initio modeling are typically less reliable than structures obtained using empirical (e.g., NMR spectroscopy or X-ray crystallography) or semi-empirical (e.g., homology modeling) techniques. However, such structures will generally be of sufficient quality, although less preferred, for use in the methods of this invention.

Indeed, in the example of M. Jannaschii described below, the AARS protein structure was obtained computationally by combining the STRUCTFAST structure alignment predicting with molecular dynamics using a force field. In some cases, a homologous protein can be used in the design, and mutations can be translated back into the protein of interest according to the sequence alignment between the two proteins.

Although the above discussion uses protein as an illustrative example, other molecules (such as small chemical compounds with less than 5000 kDa) may also be similarly modeled using art-recognized molecular modeling techniques. For additional details of predicting 3D structure, see section B below.

B. Methods for Predicting 3D Structure based on Sequence Homology

For proteins that have not been crystallized or been the focus of other structural determinations, a computer-generated molecular model of the protein and its potential binding site(s) can nevertheless be generated using any of a number of techniques available in the art. For example, the C_{α}-carbon positions of a protein sequence of interest can be mapped to a particular coordinate pattern of a protein (“known protein”) having a similar sequence and deduced structure using homology modeling techniques, and the structure of the protein of interest and velocities of each atom calculated at a simulation temperature (To) at which a docking simulation with an amino acid analog is to be determined. Typically, such a protocol involves primarily the prediction of side-chain conformations in the modeled protein of interest, while assuming a main-chain trace taken from a tertiary structure, such as provided by the known protein. Computer programs for performing energy minimization routines are commonly used to generate molecular models. For example, both the CHARMM (Brooks et al. (1983) J Comput Chem 4:187-217) and AMBER (see, Cornell et al., J. Amer. Chem. Soc. 1995, 117:5179; Woods et al., J. Phys. Chem. 1995, 99:3832-3846; Weiner et al., J. Comp. Chem. 1986, 7:230; and Weiner et al., J. Amer. Chem. Soc. 1984, 106:765) algorithms handle all of the molecular system setup, force field calculation, and analysis (see also, Eisenfield et al. (1991) Am J Physiol 261:C376-386; Lybrand (1991) J Pharm Belg 46:49-54; Froimowitz (1990) Biotechniques 8:640-644; Burbam et al. (1990) Proteins 7:99-111; Pedersen (1985) Environ Health Perspect 61:185-190; and Kini et al. (1991) J Biomol Struct Dyn 9:475-488). At the heart of these programs is a set of subroutines that, given the position of every atom in the model, calculate the total potential energy of the system and the force on each atom. These programs may utilize a starting set of atomic coordinates, the parameters for the various terms of the potential energy function, and a description of the molecular topology (the covalent structure). Common features of such molecular modeling methods include: provisions for handling hydrogen bonds and other constraint forces; the use of periodic boundary conditions; and provisions for occasionally adjusting positions, velocities, or other parameters in order to maintain or change temperature, pressure, volume, forces of constraint, or other externally controlled conditions.

Most conventional energy minimization methods use the input coordinate data and the fact that the potential energy function is an explicit, differentiable function of Cartesian coordinates, to calculate the potential energy and its gradient (which gives the force on each atom) for any set of atomic positions. This information can be used to generate a new set of coordinates in an effort to reduce the total potential energy and, by repeating this process over and over, to optimize the molecular structure under a given set of external conditions. These energy minimization methods are routinely applied to molecules similar to the subject proteins.

In general, energy minimization methods can be carried out for a given temperature, Ti, which may be different than the docking simulation temperature, To. Upon energy minimization of the molecule at Ti, coordinates and velocities of all the atoms in the system are computed. Additionally, the normal modes of the system are calculated. It will be appreciated by those skilled in the art that each normal mode is a collective, periodic motion, with all parts of the system moving in phase with each other, and that the motion of the molecule is the superposition of all normal modes. For a given temperature, the mean square amplitude of motion in a particular mode is inversely proportional to the effective force constant for that mode, so that the motion of the molecule will often be dominated by the low frequency vibrations.

After the molecular model has been energy minimized at Ti, the system is “heated” or “cooled” to the simulation temperature, To, by carrying out an equilibration run where the velocities of the atoms are scaled in a step-wise manner until the desired temperature, To, is reached. The system is further equilibrated for a specified period of time until certain properties of the system, such as average kinetic energy, remain constant. The coordinates and velocities of each atom are then obtained from the equilibrated system.

Further energy minimization routines can also be carried out. For example, a second class of methods involves calculating approximate solutions to the constrained EOM for the protein. These methods use an iterative approach to solve for the Lagrange multipliers and, typically, only need a few iterations if the corrections required are small. The most popular method of this type, SHAKE (Ryckaert et al. (1977) J Comput Phys 23:327; and Van Gunsteren et al. (1977) Mol Phys 34:1311) is easy to implement and scales as O(N) as the number of constraints increases. Therefore, the method is applicable to macromolecules. An alternative method, RATTLE (Anderson (1983) J Comput Phys 52:24) is based on the velocity version of the Verlet algorithm. Like SHAKE, RATTLE is an iterative algorithm and can be used to energy minimize the model of a subject protein.

For predicting membrane protein structures, Vaidehi et al. (PNAS USA 99: 12622-7, 2002, incorporated herein by reference) describe a program called MembStruk, which has been used to successfully predict the structure of multi-pass membrane proteins, such as G-Protein Coupled Receptors (GPCR) including adrenergic receptors (AR), olfactory receptors (OR), sweet receptors (SR), endothelial differentiation gene (EDG), etc.

C. Active Ligand Conformation Determination

Generally, structure and favorable conformations of the active ligand can be obtained using various art-recognized methods, such as obtaining the structure directly from crystallographic databases, or energy calculation for AL in solution using quantum mechanics (QM). Alternatively, any force field with molecular mechanics may be used for this calculation.

In the AARS-analog case, the favorable conformations of the analog can first be determined by generating various rotamers of the ligand over a grid of dihedral angles, followed by calculating their energies in solution using QM. Alternatively, this step can be carried out using a force field with molecular mechanics.

As is known in the art, each amino acid side chain has a set of possible conformers, called rotamers. See Ponder, et al., Acad. Press Inc. (London) Ltd. pp. 775-791 (1987); Dunbrack, et al., Struc. Biol. 1(5): 334-340 (1994); Desmet, et al., Nature 356: 539-542 (1992), all of which are hereby expressly incorporated by reference in their entirety. There are two general types of rotamer libraries: backbone dependent and backbone independent. Either type of library can be used at any position. See below (in the SCWRL section for rotamer: libraries).

In addition, a preferred embodiment does a type of “fine tuning” of the rotamer library by expanding the possible χ angle values of the rotamers by plus and minus one standard deviation (±1 SD) (or more) about the mean value, in order to minimize possible errors that might arise from the discreteness of the library. This is particularly important for aromatic residues, and fairly important for hydrophobic residues, due to the increased requirements for flexibility in the core and the rigidity of aromatic rings; it is not as important for the other residues. Thus a preferred embodiment expands the χ1 and χ2 angles for all amino acids except Met, Arg and Lys. For the intended amino acid analogs, the χ1 and χ2 angles are expanded as such in their corresponding rotamers.

To roughly illustrate the numbers of rotamers, in one version of the Dunbrack & Karplus backbone-dependent rotamer library, Ala has 1 rotamer, Gly has 1 rotamer, Arg has 55 rotamers, The has 9 rotamers, Lys has 57 rotamers, Glu has 69 rotamers, Asn has 54 rotamers, Asp has 27 rotamers, Trp has 54 rotamers, Tyr has 36 rotamers, Cys has 9 rotamers, Gln has 69 rotamers, His has 54 rotamers, Val has 9 rotamers, Ile has 45 rotamers, Leu has 36 rotamers, Mat has 21 rotamers, Ser has 9 rotamers, and Phe has 36 rotamers.

As will be appreciated by those in the art, other rotamer libraries with all dihedral angles staggered can be used or generated.

Alternatively, analog rotamers can be derived from natural amino acids. For all the natural amino acids, the possible χ1 and χ2 angles are derived from database analysis. Since this is not feasible in the case of artificial amino acids or analogs, the closest approximation for χ1 and χ2 angles for the analogs are taken to be the same as those for the natural substrate amino acid. Moreover, we would like the analogs to have a conformation as close as possible, yet with certain flexibility, to the orientation of the natural substrate amino acid in the binding pocket. So allowing similar torsional angles for the analogs as of natural substrate seems logical.

Preferably, as described above, both the χ1 and χ2 torsional angles for the analogs are varied to match those of the natural substrate rotamers in the standard backbone independent rotamer library. The torsional angles of the natural substrate in the crystal structure are also included in the new rotamer libraries for both the natural substrate and the analogs.

Alternatively, the ligand conformation can be calculated using quantum mechanics theory, using any of the software packages implementing QM. One of the many comparable programs that can be used in this embodiment is Jaguar (Schrodinger, Portland, Oreg.).

Jaguar is an extremely fast electronic structure package designed to increase the speed of ab initio calculations in order to accelerate basic and applied research projects and to enable calculations at a higher level of theory. Jaguar's speed and power make it possible to study larger systems than ever before, or to study many more systems than previously possible, within a reasonable timeframe.

Jaguar's fast computational engine allows one to address real-world problems with chemical accuracy in time periods short enough to be relevant in a fast-paced research environment requiring computational treatment at the quantum mechanical level. Jaguar implements accurate solvation energies with the improved SCRF Model.

As a well-known in the art, obtaining accurate geometries and solvation energies of molecules in solution is of great importance to many research applications. Force field based methods have often proven insufficient for the detailed description of the solvent cavity; hence a quantum mechanical description is necessary. Because the use of explicit solvent molecules is prohibitively expensive, the self-consistent reaction field (SCRF) model has become a standard approach. Using an improved SCRF model, solvation calculations in Jaguar produce a realistic description of the shape of the dielectric cavity, provide much improved accuracy for free energy of solvation, and determine the correct structure of molecules in salvation. Specifically, Jaguar calculates solvation free energies with chemical accuracy (average rms of 0.36 kcal/mol), and can thus be effectively used in calculations of the instant invention.

Jaguar can also be used to predict conformational energy differences. As is well-known in the art, finding low-energy conformers of a structure is an important step in applications such as ligand-receptor binding and identifying intra-molecular hydrogen bonds. While this has largely been a molecular mechanics computational area, it is well known that force field calculations frequently cannot reliably determine accurate conformational energies for novel scaffolds; are often missing parameters or handle non-standard or exotic atom types in a poor manner; and do not include polarization effects. Using high-level theory, Jaguar's computational efficiency makes it possible to routinely calculate the conformational energy of every important low-energy conformer of a structure; to provide an accurate determination of the geometries of and energy differences between conformers; and to obtain chemical accuracy when compared to experimental data.

In addition, Jaguar can also be run in parallel over a user-defined number of processors to further speed up calculations. Jaguar also has a highly-accurate pKa Predictor. Thus, Jaguar provides accurate determination of solute geometries and their solvation free energies, chemical accuracy in calculating conformational energy differences. For more details of the Jaguar program, see the Jaguar website (http://www.schrodinger.com/Products/jaguar.html) and Vacek, Perry, Langlois, Chem. Phys. Lett. 310: 189, 1999.

Finally, the ligand conformation may also be obtained using any suitable force field with molecular mechanics, such as the AMBER package of molecular simulation programs, which includes:
sander: Simulated annealing with NMR-derived energy restraints. This allows for NMR refinement based on NOE-derived distance restraints, torsion angle restraints, and penalty functions based on chemical shifts and NOESY volumes. Sander is also the “main” program used for molecular dynamics (MD) simulations.
gibbs: This program includes free energy perturbation (FEP) and thermodynamic integration (TI) [window growth, slow growth, and dynamically modified windows], and also allows potential of mean force (PMF) calculations.
roar: This module was contributed by Ken Merz' group at Penn State. It allows mixed quantum-mechanical/molecular-mechanical (QM/MM) calculations, “true” Ewald simulations, and alternate molecular dynamics integrators.
nmode: Normal mode analysis program using first and second derivative information, used to find search for local minima, perform vibrational analysis, and search for transition states.
LEaP: LEaP is an X-windows-based program that provides for basic model building and AMBER coordinate and parameter/topology input file creation. It includes a molecular editor which allows for building residues and manipulating molecules. Motif-style X-windows Athena widgets and a table widget used in LEaP were written by Vladimir Romanovski.
antechamber: This program suite automates the process of developing force field descriptors for most organic molecules. It starts with structures (usually in PDB format), and generates files that can be read into LEaP for use in molecular modeling. The force field description that is generated is designed to be compatible with the usual Amber force fields for proteins and nucleic acids.
ptraj and carnal: These are programs to analyze MD trajectories, computing a variety of things, like RMS deviation from a reference structure, hydrogen bonding analysis, time-correlation functions, diffusional behavior, and so on.
mm_pbsa: This is a script to automate post-processing of MD trajectories, to analyze energetics using continuum solvent ideas. It can be used to break energies into “pieces” arising from different residues, and to estimate free energy differences between conformational basins.

It should be understood that these are but a few illustrative examples of many possible alternative QM or MM programs that can be used with the instant invention, and is by no means limiting. Many similar programs, with or without minor modifications, can also be adapted for use in the instant invention.

D. Alternative Docking Methods

Aside from the HIERDOCK program described above, there are a variety of computational methods that can be readily adapted for docking ALs (amino acid analogs) that would have appropriate steric and electronic properties to interact with the binding site of the target protein (such as an AARS). See, for example, Cohen et al. (1990) J. Med. Cam. 33: 883-894; Kuntz et al. (1982) J. Mol. Biol. 161: 269-288; DesJarlais (1988) J. Med. Cam. 31: 722-729; Bartlett et al. (1989) (Spec. Publ., Roy. Soc. Chem.) 78: 182-196; Goodford et al. (1985) J. Med. Cam. 28: 849-857; DesJarlais et al. J. Med. Cam. 29: 2149-2153). Directed methods generally fall into two categories: (1) design by analogy in which 3-D structures of known molecules (such as from a crystallographic database) are docked to the binding site structure and scored for goodness-of-fit; and (2) de novo design, in which the analog model is constructed piece-wise in the binding site. The latter approach, in particular, can facilitate the development of novel molecules, uniquely designed to bind to the target protein (e.g., an AARS mutant) binding site.

In an illustrative embodiment, the design of potential analogs that may function with a particular target protein begins from the general perspective of shape complimentary for the binding site of the target protein, and a search algorithm is employed which is capable of scanning a database of small molecules of known three-dimensional structure for candidates which fit geometrically into the substrate binding site. Such libraries can be general small molecule libraries, or can be libraries directed to amino acid analogs or small molecules which can be used to create amino acid analogs. It is not expected that the molecules found in the shape search will necessarily be leads themselves, since no evaluation of chemical interaction necessarily be made during the initial search. Rather, it is anticipated that such candidates might act as the framework for further design, providing molecular skeletons to which appropriate atomic replacements can be made. Of course, the chemical complimentary of these molecules can be evaluated, but it is expected that atom types will be changed to maximize the electrostatic, hydrogen bonding, and hydrophobic interactions with the substrate binding site. Most algorithms of this type provide a method for finding a wide assortment of chemical structures that may be complementary to the shape of the target protein binding pocket.

For instance, each of a set of small molecules from a particular data-base, such as the Cambridge Crystallographic Data Bank (CCDB) (allen et al. (1973) J. Chem. Doc. 13: 119), is individually docked to the binding site of the target protein in a number of geometrically permissible orientations with use of a docking algorithm. In a preferred embodiment, a set of computer algorithms called DOCK, can be used to characterize the shape of invaginations and grooves that form the binding site. See, for example, Kuntz et al. (1982) J. Mol. Biol. 161: 269-288. The program can also search a database of small molecules for templates whose shapes are complementary to particular binding site of the target protein. Exemplary algorithms that can be adapted for this purpose are described in, for example, DesJarlais et al. (1988) J Med Chem 31:722-729.

The orientations are evaluated for goodness-of-fit and the best are kept for further examination using molecular mechanics programs, such as AMBER or CHARMM, etc. Such algorithms have previously proven successful in finding a variety of molecules that are complementary in shape to a given binding site of a receptor or enzyme, and have been shown to have several attractive features. First, such algorithms can retrieve a remarkable diversity of molecular architectures. Second, the best structures have, in previous applications to other proteins, demonstrated impressive shape complementarity over an extended surface area. Third, the overall approach appears to be quite robust with respect to small uncertainties in positioning of the candidate atoms.

In certain embodiments, the subject method can utilize an algorithm described by Goodford (1985, J Med Chem 28:849-857) and Boobbyer et al (1989, J Med Chem 32:1083-1094). Those papers describe a computer program (GRID) which seeks to determine regions of high affinity for different chemical groups (termed probes) on the molecular surface of the binding site. GRID hence provides a tool for suggesting modifications to known ligands that might enhance binding. It may be anticipated that some of the sites discerned by GRID as regions of high affinity correspond to “pharmacophoric patterns” determined inferentially from a series of known ligands. As used herein, a pharmacophoric pattern is a geometric arrangement of features of the anticipated amino acid analog that is believed to be important for binding. Goodsell and Olson (1990, Proteins: Struct Funct Genet 8:195-202) have used the Metropolis (simulated annealing) algorithm to dock a single known ligand into a target protein, and their approach can be adapted for identifying suitable amino acid analogs for docking with the AARS binding site. This algorithm can allow torsional flexibility in the amino acid sidechain and use GRID interaction energy maps as rapid lookup tables for computing approximate interaction energies.

Yet a further embodiment of the present invention utilizes a computer algorithm such as CLIX which searches such databases as CCDB for small molecules which can be oriented in the substrate binding site of the target protein in a way that is both sterically acceptable and has a high likelihood of achieving favorable chemical interactions between the candidate molecule and the surrounding amino acid residues. The method is based on characterizing the substrate binding site in terms of an ensemble of favorable binding positions for different chemical groups and then searching for orientations of the candidate molecules that cause maximum spatial coincidence of individual candidate chemical groups with members of the ensemble. The current availability of computer power dictates that a computer-based search for novel ligands follows a breadth-first strategy. A breadth-first strategy aims to reduce progressively the size of the potential candidate search space by the application of increasingly stringent criteria, as opposed to a depth-first strategy wherein a maximally detailed analysis of one candidate is performed before proceeding to the next. CLIX conforms to this strategy in that its analysis of binding is rudimentary—it seeks to satisfy the necessary conditions of steric fit and of having individual groups in “correct” places for bonding, without imposing the sufficient condition that favorable bonding interactions actually occur. A ranked “shortlist” of molecules, in their favored orientations, is produced which can then be examined on a molecule-by-molecule basis, using computer graphics and more sophisticated molecular modeling techniques. CLIX is also capable of suggesting changes to the substituent chemical groups of the candidate molecules that might enhance binding. Again, the starting library can be of amino acid analogs or of molecules which can be used to generate the sidechain of an amino acid analog.

The algorithmic details of CLIX is described in Lawerence et al. (1992) Proteins 12:31-41, and the CLIX algorithm can be summarized as follows. The GRID program is used to determine discrete favorable interaction positions (termed target sites) in the binding site of the target protein for a wide variety of representative chemical groups. For each candidate ligand in the CCDB an exhaustive attempt is made to make coincident, in a spatial sense in the binding site of the protein, a pair of the candidate's substituent chemical groups with a pair of corresponding favorable interaction sites proposed by GRID. All possible combinations of pairs of ligand groups with pairs of GRID sites are considered during this procedure. Upon locating such coincidence, the program rotates the candidate ligand about the two pairs of groups and checks for steric hindrance and coincidence of other candidate atomic groups with appropriate target sites. Particular candidate/orientation combinations that are good geometric fits in the binding site and show sufficient coincidence of atomic groups with GRID sites are retained.

Consistent with the breadth-first strategy, this approach involves simplifying assumptions. Rigid protein and small molecule geometry is maintained throughout. As a first approximation rigid geometry is acceptable as the energy minimized coordinates of the binding site of the AARS mutant, describe an energy minimum for the molecule, Albeit a local one.

A further assumption implicit in CLIX is that the potential ligand, when introduced into the substrate binding site of the target protein, does not induce change in the protein's stereochemistry or partial charge distribution and so alter the basis on which the GRID interaction energy maps were computed. It must also be stressed that the interaction sites predicted by GRID are used in a positional and type sense only, i.e., when a candidate atomic group is placed at a site predicted as favorable by GRID, no check is made to ensure that the bond geometry, the state of protonation, or the partial charge distribution favors a strong interaction between the protein and that group. Such detailed analysis should form part of more advanced modeling of candidates identified in the CLIX shortlist.

Yet another embodiment of a computer-assisted molecular design method for identifying amino acid analogs that may be utilized by a predetermined AARS mutant comprises the de novo synthesis of potential inhibitors by algorithmic connection of small molecular fragments that will exhibit the desired structural and electrostatic complementarity with the substrate binding site of the enzyme. The methodology employs a large template set of small molecules with are iteratively pieced together in a model of the AARS′ substrate binding site. Each stage of ligand growth is evaluated according to a molecular mechanics-based energy function, which considers van der Waals and Coulombic interactions, internal strain energy of the lengthening ligand, and desolvation of both ligand and enzyme. The search space can be managed by use of a data tree which is kept under control by pruning according to the binding criteria.

In yet another embodiment, potential amino acid analogs can be determined using a method based on an energy minimization-quenched molecular dynamics algorithm for determining energetically favorable positions of functional groups in the substrate binding site of a mutant AARS enzyme. The method can aid in the design of molecules that incorporate such functional groups by modification of known amino acid and amino acid analogs or through de novo synthesis.

For example, the multiple copy simultaneous search method (MCSS) described by Miranker et al. (1991) Proteins 11: 29-34 can be adapted for use in the subject method. To determine and characterize a local minima of a functional group in the force field of the protein, multiple copies of selected functional groups are first distributed in a binding site of interest on the AARS protein. Energy minimization of these copies by molecular mechanics or quenched dynamics yields the distinct local minima. The neighborhood of these minima can then be explored by a grid search or by constrained minimization. In one embodiment, the MCSS method uses the classical time dependent Hartee (TDH) approximation to simultaneously minimize or quench many identical groups in the force field of the protein.

Implementation of the MCSS algorithm requires a choice of functional groups and a molecular mechanics model for each of them. Groups must be simple enough to be easily characterized and manipulated (3-6 atoms, few or no dihedral degrees of freedom), yet complex enough to approximate the steric and electrostatic interactions that the functional group would have in substrate binding to the site of the AARS protein. A preferred set is, for example, one in which most organic molecules can be described as a collection of such groups (Patai's Guide to the Chemistry of Functional Groups, ed S. Patai (New. York: John Wiley, and Sons, (1989)). This includes fragments such as acetonitrile, methanol, acetate, methyl ammonium, dimethyl ether, methane, and acetaldehyde.

Determination of the local energy minima in the binding site requires that many starting positions be sampled. This can be achieved by distributing, for example, 1,000-5,000 groups at random inside a sphere centered on the binding site; only the space not occupied by the protein needs to be considered. If the interaction energy of a particular group at a certain location with the protein is more positive than a given cut-off (e.g. 5.0 kcal/mole) the group is discarded from that site. Given the set of starting positions, all the fragments are minimized simultaneously by use of the TDH approximation (Elber et al. (1990) J Am Chem Soc 112: 9161-9175). In this method, the forces on each fragment consist of its internal forces and those due to the protein. The essential element of this method is that the interactions between the fragments are omitted and the forces on the protein are normalized to those due to a single fragment. In this way simultaneous minimization or dynamics of any number of functional groups in the field of a single protein can be performed.

Minimization is performed successively on subsets of, e.g. 100, of the randomly placed groups. After a certain number of step intervals, such as 1,000 intervals, the results can be examined to eliminate groups converging to the same minimum. This process is repeated until minimization is complete (e.g. RMS gradient of 0.01 kcal/mole/Å). Thus the resulting energy minimized set of molecules comprises what amounts to a set of disconnected fragments in three dimensions representing potential sidechains for amino acid analogs.

The next step then is to connect the pieces with spacers assembled from small chemical entities (atoms, chains, or ring moieties) to form amino acid analogs, e.g., each of the disconnected can be linked in space to generate a single molecule using such computer programs as, for example, NEWLEAD (Tschinke et al. (1993) J Med Chem 36: 3863,3870). The procedure adopted by NEWLEAD executes the following sequence of commands (1) connect two isolated moieties, (2) retain the intermediate solutions for further processing, (3) repeat the above steps for each of the intermediate solutions until no disconnected units are found, and (4) output the final solutions, each of which is single molecule. Such a program can use for example, three types of spacers: library spacers, single-atom spacers, and fuse-ring spacers. The library spacers are optimized structures of small molecules such as ethylene, benzene and methylamide. The output produced by programs such as NEWLEAD consist of a set of molecules containing the original fragments now connected by spacers. The atoms belonging to the input fragments maintain their original orientations in space. The molecules are chemically plausible because of the simple makeup of the spacers and functional groups, and energetically acceptable because of the rejection of solutions with van-der Waals radii violations.

In addition, the order in which the steps of the present method are performed is purely illustrative in nature. In fact, the steps can be performed in any order or in parallel, unless otherwise indicated by the present disclosure.

Furthermore, the method of the present invention may be performed in either hardware, software, or any combination thereof, as those terms are currently known in the art. In particular, the present method may be carried out by software, firmware, or microcode operating on a computer or computers of any type. Additionally, software embodying the present invention may comprise computer instructions in any form (e.g., source code, object code, interpreted code, etc.) stored in any computer-readable medium (e.g., ROM, RAM, magnetic media, punched tape or card, compact disc (CD) in any form, DVD, etc.). Furthermore, such software may also be in the form of a computer data signal embodied in a carrier wave, such as that found within the well-known Web pages transferred among devices connected to the Internet. Accordingly, the present invention is not limited to any particular platform, unless specifically stated otherwise in the present disclosure.

Exemplary computer hardware means suitable for carrying out the invention can be a Silicon Graphics Power Challenge server with 10 R1000 processors running in parallel.

E. Alternative Force Fileds

Although the DREIDING Force Field (see Mayo et al., J. Phys. Chem. 1990, 94:8897) is chosen an illustrative force filed for use in this procedure, other force fields, such as AMBER (see, Cornell et al, J. Amer. Chem. Soc. 1995, 117:5179; Woods et al., J. Phys. Chem. 1995, 99:3832-3846; Weiner et al., J. Comp. Chem. 1986, 7:230; and Weiner et al., J. Amer. Chem. Soc. 1984, 106:765); AMBER/OPLS (Damm et al., J. Comp. Chem. 18: 1955-1970, 1997; Jorgensen et al., J. Am. Chem. Soc. 118: 11225-11236, 1996; Jorgensen & Tirado-Rives, J., J. Am. Chem. Soc. 110: 1657-1666, 1998; Kaminski et al., J. Phys. Chem. 98: 13077-13082, 1994); CHARMM (Brooks et al. J Comput Chem 4:187-217, 1983; Feller et al., Biophys. J. 73, 2269-2279, 1997; MacKerell et al., J. Phys. Chem., B 102: 3586-3617, 1998; Mackerell et al., J. Amer. Chem. Soc. 117: 11946-11975, 1995; Momany & Rone J. Comp. Chem. 13: 888-900, 1992; Pavelites et al., J. Comp. Chem. 18, 221-239, 1997; Schlenkrich et al. Empirical Potential Energy Function for Phospholipids: Criteria for Parameter Optimization and Applications in “Biological Membranes: A Molecular Perspective from Computation and Experiment,” K. M. Merz and B. Roux, Eds. Birkhauser, Boston, pp 31-81, 1996); Discover (cvff-Dauber-Osguthorpe, P. et al., Proteins: Structure, Function and Genetics, 4, 31-47, 1988; cff-Hagler, A. T.; Ewig, C. S. “On the use of quantum energy surfaces in the derivation of molecular force fields”, Comp. Phys. Comm. 84, 131-155, 1994; Hwang, M.-J. et al., J. Amer. Chem. Soc. 116: 2515-2525, 1994; Maple, J. R. et al, J. Comput. Chem. 15: 162-182, 1994; Maple, J. R. et al., Israel J. Chem. 34: 195-231, 1994); ECEPP/2 (Momany et al., J. Phys. Chem. 79: 2361-2381, 1975; Nemethy et al., J. PHys. Chem. 87: 1883-1887, 1983; Sippl et al., J. Phys. Chem. 88: 6231-6633, 1984); GROMOS (Hermans, J. et al., Biopolymers 23: 1, 1984; Ott and Meyer J Comp Chem 17: 1068-1084, 1996; van Gunsteren et al., “The GROMOS force field” in Encyclopaedia of Computational Chemistry, 1997); MM2 (allinger J. Am. Chem. Soc. 99: 8127-8134, 1997; allinger et al., J. Comp. Chem. 9: 591-595, 1988; Lii et al., J. Comp. Chem. 10: 503-513, 1989); MM3 (allinger et al., J. Am. Chem. Soc. 111: 8551-8565, 1989; Hay et al., J. Molecular Structure 428: 203-219, 1998; Lii & allinger J. Am. Chem. Soc. 111: 8566-8575, 1989; Lii & allinger J. Am. Chem. Soc. 111: 8576-8582, 1989; Lii & allinger J. Comp. Chem. 12: 186-199, 1991; Lii & allinger J. Comp. Chem. 19: 1001-1016, 1998); MM4 (allinger et al., J. Comp. Chem. 17: 642-668, 1996; allinger et al., J. Comp. Chem. 17: 747-755, 1996; allinger and Fan, J. Comp. Chem. 18: 1827-1847, 1997; Nevens et al., J. Comp. Chem. 17:669-694, 1996; Nevins et al., J. Comp. Chem. 17: 695-729, 1996; Nevins and allinger, J. Comp. Chem. 17-730-746, 1996); MMFF94 (Halgren J. Am. Chem. Soc. 114: 7827-7843, 1992; Halgren J. Comp. Chem. 17, 490-519; 1996; Halgren J. Comp. Chem. 17: 520-552, 1996; Halgren J. Comp. Chem. 17: 553-586, 1996; Halgren and Nachbar J. Comp. Chem. 17: 587-615, 1996; Halgren J. Comp. Chem. 17: 616-641, 1996); Tripos (Clark et al., J. Comp. Chem. 10: 982-1012, 1989); Comparisons and Evaluations (Engler et al., J. Am. Chem. Soc. 95: 8005-8025, 1973; Gundertofte et al., J. Comp. Chem. 12: 200-208, 1991; Gundertofte et al., J. Comp. Chem. 17: 429-449, 1996; Hall and Pavitt, J. Comp. Chem. 5: 441-450, 1984; Hobza et al., J. Comp. Chem. 18: 1136-1150, 1997; Kini et al., J. Biomol. Structure and Dynamics 10: 265-279, 1992; Roterman et al., J. Biomol. Struct. and Dynamics 7: 391-419, 1989; Roterman et al., J. Biomol. Struct. and Dynamics 7: 421-452, 1989; Whitlow and Teeter, J. Am. Chem. Soc. 108: 7163-7172, 1989); AMBER94; MMFF; MMFFs; OPLS (Kaminski et al., J. Phys. Chem. B 105: 6474-6487, 2001); OPLS-AA (Jorgensen et al., JACS 118: 11225, 1996); UFF (Rappe et al., J. Am. Chem. Soc. 114: 10024, 1992; Casewit et al., J. Am. Chem. Soc. 114: 10035, 1992; Casewit et al., J. Am. Chem. Soc. 114: 10046, 1992; Rappe et al., Inorg. Chem. 32: 3438, 1993), etc., can also be used to calculate clashes and binding energies.

In addition, the functional forms of the non-bond energy in Equation 1 can have different forms. The dielectric constant in the Coulomb term can be distance dependent. The charges for both the protein and the ligand can be varied. This includes charges from experiment, or charges based on various models, such as, but not limited to, QEq (“Charge Equilibration,” see Rappe and Goddard, J. Phys. Chem. 95: 3358-63, 1991), Del Re (Del Re J. Chem. Soc. 4031, 1958; Poland and Scheraga Biochemistry 6: 3791, 1967; Coefficients modified in MMP to take into account pi contributions—values can be modified by the user), Gasteiger (or PEOE, see Gasteiger and Marsili, Tetrahedron 36: 3219-28, 1980), MPEOE (DQP) (No et al., J. Phys. Chem. 94: 4732, 1990; No et al., J. Phys. Chem. 94: 4740, 1990; Park et al., J. Comp. Chem. 14:1482, 1993), ReaxFF (Goddard et al., “The ReaxFF Polarizable Reactive Force Fields for Molecular Dynamics Simulation of Ferroelectrics,” Presented at Fundamental Physics of Ferroelectrics 2002, held in Washington D. C., Feb. 3-6, 2000, Chairmen: Ron Cohen and Takeshi Egami), etc. The van der Waals term can have different forms, such as a Morse potential (U(x)=Dₑ[1−e^{-(x-r)}ₑ]²) instead of a Leonard-Jones potential. The 6-12 Leonard-Jones potential can be made 6-10, or even softer to allow closer contact. Finally the hydrogen bond term can have several different variations. The three body form is used in Equation 1, but two-body or four body form is common too, and can also be similarly used. Further, in some force fields, hydrogen bond can also be treated implicitly as part of the Coulomb term.

F. Alternative Solvation Methods

Solvation is an important factor in determining biomolecular stability and binding properties. As an integrated part in the COP design, implicit solvent is used to minimize the structures and to calculate binding energies. These implicit solvent model includes, but not limited to Surface Generalized Born (SGB) model (Ghosh et al. J. Phys. Chem. 102: 10983-10990, 1998), Solvent Accessible Surface Area (SASA)/Analytical Volume Generalized Born (AVGB) model (Zamanakos, Ph.D. Thesis, Calif. Institute of Technology, Pasadena, Calif., 2001), and Poisson-Boltzmann (PB) model.

In addition, explicit solvent molecules can be easily added to the calculation of binding energy, as long as the solvation model is accurate enough to account for the solvation effect of ligand binding to the target protein. This can usually be used in the evaluation of the best cases for final selection in the design.

Many commercial software packages support calculations for various explicit and/or implicit solvation models, such as Impact, a molecular mechanics program specifically designed to handle large macromolecular simulations. The program effortlessly treats simulations on ligand-protein systems in salvation or in vacuo, enabling the study of large systems in a timely manner. For example, Impact's explicit solvation offers the possibility of setting up calculations in solvent boxes, where water or user-defined solvent molecules are placed at distinct sites of a solvent box. Periodic boundary conditions are also available. Continuum models greatly enhance computational speed. Impact's continuum solvation method is a surface area version of the generalized Bom model (SGB). The SGB model has been shown to give significant improvement in accuracy over the uncorrected GB model. The large number of solvent molecules used in solution-phase simulations is also avoided using the PBF (Poisson Boltzmann solver) solvation model in Impact (Cortis and Friesner J. Comp. Chem. 18: 1570, 1997; Cortis and Friesner J. Comp. Chem. 18: 1591, 1997). This model offers additional savings in computational expense using a customizable finite element mesh.

Another exemplary software package that employs multiple force fields and matching salvation methods is the MacroModel® program from Schrodinger (Portland, Oreg.).

G. Alternative Binding Energy Calculation

It is always important to get the correct relative binding energies for different ligands. As an alternative method, minimization in the Potential of Mean Force (PMF) from implicit continuum solvent model can also be used to calculate binding (free) energies. As another alternative, the average dynamic free energy instead of free energy form a single conformation can be used. In fact, for the exemplary case of amino acids binding to AARS, is was found that the binding energy calculation with PMF is very close to experimental numbers when the average dynamic free energy is used.

In addition, other methods may (at least in theory) be used to calculate binding free energies, although they may require significantly longer computation time. These methods include Free Energy Perturbation (FEP) (Becker et al., Marcel Dekker, New York, 2001), and methods based on thermodynamic cycles. See the AMBER package described in the section above. Obviously, such methods can be more realistically implemented with a faster and more powerful computer.

H. Protein Side-Chain Modeling

There are several side chain modeling methods that can be incorporated into the COP procedure. These side chain modeling methods include SCWRL, SCAP, and methods based on branch-and-bound, dead-end-elimination algorithms.

SCRWL (Sidechain Placement with a Rotamer Library):

SCWRL (Bower et al., J. Mol. Biol. 267: 1268-82, 1997) is a program for adding side-chains to a protein backbone based on the backbone-dependent rotamer library. The library provides lists of chi1-chi2-chi3-chi4 values and their relative probabilities for residues at given phi-psi values, and explores these conformations to minimize sidechain-backbone clashes and sidechain-sidechain clashes. It is possible to get output from the program at any of the three steps: best library rotamers, no clashes relieved; backbone clashes relieved; backbone and sidechain-sidechain clashes relieved.

The present version of the program (scwr12.7) achieves a prediction rate for chi1 dihedral angles correct within 40 degrees for all residues of 81.0%.

The method used by SCWRL is based on the hypothesis that a great deal of the information needed for sidechain positioning is contained in the local mainchain conformation of each residue, but that a search strategy to resolve steric exclusions is also necessary for the most accurate predictions. SCWRL is a single self-contained program optimized for speed and accuracy. SCWRL is designed to take full advantage of the rotamer approximation and the strong backbone dependencies rotamers display to create an initial placement for each residue, followed by systematic searches to resolve steric clashes.

It begins with the mainchain atoms N, C_{α}, C, and O from a protein structure. The mainchain dihedral angles phi and psi calculated by SCWRL, together with the sequence, are used to look up an ordered list of rotamers for each residue from a rotamer database. Each of these potential rotamers is built, using bond lengths and angles from the AMBER 4.1 parameter set, and the set of rotamers is searched for the minimum steric clash to create the output structure.

The vdw energy function in SCWRL is a simple repulsive steric clash check. For a pair of atoms, the energy of interaction is given by:
E=0.0 R>R0
E=(R/R0)(−57.273)+57.273 R0>=R>=0.83R0 Eq. A)
E=10.0 R<0.83R0

where R is the distance between the atoms and R0 is the sum of their radii. The linear portion of the function approximates the repulsive curve of a Leonard-Jones potential. A graph showing the energy function used by SCWRL and a Leonard-Jones potential (light line) is displayed in the SCWRL website.

To make the steric term more forgiving on the rigid rotamers, the radii of atoms are reduced roughly 15% from their van der Waals radii, to values which approximate the distance where a Leonard-Jones potential would become repulsive.

Radii that can be used for steric clash checks are listed below:

Atom type

| | C | N | O | S |
|---|---|---|---|---|
| Radius(Angstroms) | 1.6 | 1.3 | 1.3 | 1.5 |

Note: radii increased to these values, SCWRL2.0 (R. Dunbrack, Aug. 21, 1997).

Each rotamer is also given a local rotamer energy based on the probability of that rotamer in the backbone-dependent rotamer library. These energies are taken from the probabilities of the backbone-dependent rotamer library, as −ln(pi/p0), where p0 is the probability of the most probably rotamer, and pi is the probability of the i-th rotamer (assuming kT=1).

SCWRL first calculates possible disulfide bonds from rotamers of cysteine residues and resulting distances between sulfur atoms. These residues are then frozen in their disulfide conformation, and the sidechain atoms are treated as part of the backbone scaffold in determining the conformations of the other residue types.

To overcome the combinatorial nature of the sidechain packing problem, the search strategy does not involve a search of every rotamer of every sidechain, but rather takes a structure with residues in their most favorable backbone-dependent rotamers and systematically resolves the conflicts that arise from that structure. Each residue begins in its most favored rotamer, according to the rotamer database used. This is the first stage structure. When a sidechain from the first stage structure has a steric clash as defined by Eq. A with the (fixed) mainchain, the rotamer for that residue is changed to progressively less favorable rotamers until one is found that does not conflict with the mainchain. The second stage structure has all of these sidechain to mainchain clashes relieved.

At this point, there will be some sidechain to sidechain clashes. Where these occur, the two clashing residues are put in a “cluster” of residues which interact. The clashing residues are allowed to explore all of their respective rotamers, save those which cause sidechain to mainchain clashes, and any residues which clash with these “rotating” residues are added to the cluster and themselves allowed to explore all their rotamers. In this way, clusters of residues grow by clashing with residues not in the original cluster. Clusters can also merge when two rotamers from residues in different clusters clash, in which case all of the residues from the two clusters become part of the same cluster. Sidechains which do not clash with the mainchain, and are never involved in a steric clash with an activated residue, are left in their most favorable backbone-dependent rotamer.

When the clusters have grown to their final size, each one represents an exclusive subset of residues which are allowed to interact with each other. Each cluster is solved, in turn, through a combinatorial search to find its minimum steric clash score. When all of the clusters have been solved, the stage three structure is output as the solution.

The search procedure tests each residue and combination of residues in the order they were added to the cluster. Rotamers for each residue are tested in order of decreasing favorability. The first combination of rotamers which is found to have a steric clash score of zero is taken as the final solution. If no such combination is found, all the rotamer combinations are searched, and the combination with the minimum steric clash score is taken as the final solution.

Occasionally, clusters grow too large to be solved quickly with a combinatorial search. When such a large number of combinations is reached, the cluster is broken into sub-clusters to speed the solution time. In our case, the limit is set for clusters that cannot be solved by the combinatorial search in approximately one second, which is reached for clusters containing more than 1.5E7 rotamer combinations, about 15 residues. A large cluster is parsed by finding the residue in that cluster whose removal from the cluster results in the smallest sub-clusters. Then each of the sub-clusters is solved in the presence of each of the “keystone” residue's potential rotamers. For example, in a 21-residue cluster where every residue has three potential rotamers, the combinations to search will number 3ˆ21, or 1.0×E10. If a residue in this cluster is found which divides the cluster into two 10-residue sub-clusters, then the combinations to search will number 3(3ˆ10+3ˆ10)=3.5×E5.

For the purposes of searching alternate conformations to resolve a cluster of clashing sidechains, residues are searched in order from high entropy sidechains to low entropy sidechains. E.g. a sidechain with p0=0.1 is searched before a sidechain with p0=0.9. Since SCWRL favors early solutions to the cluster over later ones, if the energies are equal, this is a small benefit. As before, all residues which clash in their library conformations are searched (in order of decreasing entropy) before sidechains which clash with lower probability rotamers of the original clustering residues.

This parsing of clusters into sub-clusters is a recursive process, and if the sub-clusters still contain more combinations than the cutoff, or if a keystone residue fails to break a cluster into non-interacting sub-clusters, the remaining clusters are passed down to a new level for additional parsing. Only in some very rare cases, the parse routine cannot find a subset of keystones which breaks the cluster up fast enough to overcome the combinatorics.

For more details of the SCWRL program, including licensing information, see the SCWRL website maintained by R. Dunbrack (last modified in March 2001).

The related Backbone-dependent Rotamer Library website maintained by Dunbrack (http://www.fccc.edu/research/labs/dunbrack/bbdep.html) is last updated in May 2002. As is well-known in the art, certain rotameric states will be higher in energy than others because of steric interactions that cause the sidechain to twist out of the way of neighboring atoms, inflicting a high dihedral energy on the residue. These interactions can be “backbone-independent”, that is, not depending on the conformation of the local backbone of the residue, or “backbone-dependent”, that is, depending on the local backbone conformation as determined by the backbone dihedrals phi and psi. The backbone-independent rotamer library is similar to the familiar Ponder & Richards rotamer library (J. Mol. Biol. 193: 775-791, 1987). The backbone dependent rotamer library defines a rotamer distribution for small ranges of phi and psi, basically a rotamer library for every 10×10 degree box of phi and psi.

The site mentioned above includes backbone-independent and backbone-dependent rotamer libraries and the SCWRL program for making sidechain conformation predictions from the library and input phi and psi values and for evaluating the rotamers and chi angles of a preliminary x-ray, NMR, or model structure in comparison to the experimental distributions of rotamers and chi angles in the Protein Databank.

The library is subject to frequent update (every couple of months). The May 2002 version of the library contains 850 chains from the PDB of resolution 1.7 \acute{Å} or better, and less than 40% homology with other chains in the set. The list was determined from the Dunbrack group's algorithm for finding sets of PDB chains with maximum sequence identities and resolution cutoffs. More specifically, the chains used in constructing the database are all from x-ray crystallographic structures from the Brookhaven Protein Databank (PDB). The algorithm for selecting these lists is similar to that of Hobohm and Sander. The only difference is the addition of resolution cutoffs, so that one gets the largest list possible with PDB entries of a certain resolution or better, and also that the algorithm favored higher resolution structures over lower resolution structures (by proceeding from high resolution to low resolution in the reject-until-done procedure). These lists are described on the related PISCES web site (http://www.fccc.edu/research/labs/dunbrack/pisces/).

SCAP (Xiang and Honig, J. Mol. Biol. 311: 421-430, 2001):

Scap is a program for protein side-chain prediction and residue mutation. The program can make prediction on all residues or on certain residues in a protein of multiple chains. It can automatically detect if the residue to be predicted or mutated is backbone only, or complete with all side-chain atoms. If the residue is backbone only, it will first add side-chains and then do predictions; if the residue is to be mutated, the residue is first mutated accordingly and prediction is then performed.

The scap performs side-chain prediction using coordinate rotamer libraries. There are four coordinate rotamer libraries included in the library: side_small_rotamer, side_medium_rotamer, side_large_rotamer, and side_mix_rotamer. Side_small_rotamer library has 214 side-chain rotamers with 40-degree chi angle cutoff. This is equivalent to the 214 sidechain rotamer library of Tuffery's with standard bond angle and length; side_medium_rotamer has 3222 side-chain rotamers compiled from 297 chains with 20 degree cutoff and 96% representation; side₁₃ large_rotamer has 6487 side-chain rotamers compiled from 297 chains with 10 degree cutoff and 96% representation; side-mix-rotamer is a mix of side_medium_rotamer and side_large_rotamer. It includes all rotamers from side_large_rotamer except for ARG, LYS and MET which come from side_medium_rotamer. The more coordinate rotamer and dihedral angle rotamer libraries can be downloaded from website: http://trantor.bioc.columbia.edu/˜xiang/sidechain/index.html. More detailed information about the methods and algorithms in scap can be found in the paper: Xiang Z, Honig B. Extending the accuracy limits of prediction for side-chain conformations. J. Mol. Biol. 2001 311:421-30 (incorporated herein by reference); and Xiang, Z; Honig, B. Colony energy improves prediction of exposed sidechain conformations. The software and manual can be downloaded at: http://trantor.bioc.columbia.edu/˜xiang/j ackal/index.html.

Scap support the following platform: SGI, Sun Solarius, Linux and Microsoft Window.

Finally, it should be understood that COP is a generic method that can be applied to any target protein for recognizing a desired active ligand (AL) vs. one or more structurally related inactive ligand(s) (IL). The ligand type can be any molecule that is a binding target to a protein and has some sort of anchoring point as the reaction center. In one embodiment, the ligand itself may be a protein, and thus the binding can be between two proteins. For example, if protein X normally binds protein Y, then a mutant protein X (denoted X′) can be used as a target protein in COP, such that a complimentary counterpart protein Y′ can be designed for binding to X′.

The instant invention uses in the design calculation a full force field, including hydrogen-bonding capability and solvation effects. Hence the all-atom energy function is more accurate, and also biased towards recognizing the new ligand compared to its competitors. In addition, proteins are allowed to be fully movable in the sage of binding calculation. The algorithm has been designed to make few mutations to recognize a desired ligand. The conformational search of side-chain rotamers can be exhaustive. All these features and combinations thereof allows COP to be a unique design approach.

I. The Graphic User Interface for COP

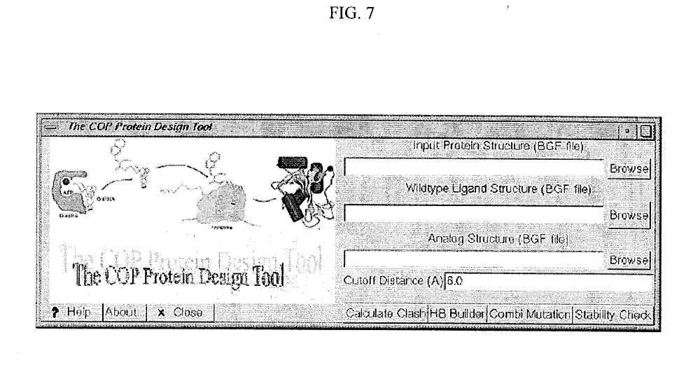

To make COP user-friendlier, a graphic user interface (GUI) can be implemented using any of the art-recognized GUI interface builder. For example, a GUI has been written for COP using Glade, a GTK-based free user interface builder. FIG. 7 shows the screen snapshot when COP is started using the graphical interface.

Clicking a button brings out a popup window with the corresponding information. For example, clicking the “About . . . ” button will open a window showing the version and copyright information of COP. The help window is designed to let user know the conventions used in the COP program. The four buttons in the bottom right of the window are for carrying four functions in COP, with each corresponding to a different program. “Calculate Clash” will run the clash identification to find mutation residues and their mutation targets to relieve clash. “HB Builder” uses a rotamer library to build possible hydrogen bond donor or acceptor residues in the binding site to stabilize new polar atoms in the analog ligand, if there is any. “Combi Mutation” carries out the combined mutation step in COP, and calculates the binding energies of each ligand including competing natural amino acids to generated mutants. A list of top candidates will be given at the end. Finally the “Stability Check” step will eliminate any mutant that potentially cannot fold correctly.

J. Exemplary Uses

To date, over 100 noncoded amino acids (all ribosomally acceptable) have been reportedly introduced into proteins using other methods (see, for example, Schultz et al., J. Am. Chem. Soc., 103: 1563-1567, 1981; Hinsberg et al., J. Am. Chem. Soc., 104: 766-773, 1982; Pollack et al., Science, 242: 1038-1040, 1988; Nowak et al., Science, 268: 439-442, 1995) all these analogs may be used for COP re-designed suitable AARSs that can preferentially and efficiently incorporate these analogs into protein products over their natural substrate amino acids. In general, the COP re-designed AARSs (mutant AARSs) of the instant invention can be used to incorporate amino acid analogs into protein products either in vitro or in vivo.

The ration of natural/analog amino acids may be controlled so that the degree of incorporation may be adjusted. In a preferred embodiment, 100% of one or more of the natural amino acids (such as Phe) may be depleted so that only its analogs are incorporated.

In another preferred embodiment, two or more analogs may be used in the samc in vitro or in vivo translation system. These analogs can be analogs of the same amino acid (for example, different Phe analogs), or different amino acids (for example, analogs of Phe and Tyr, respectively).

For in vitro use, one or more AARSs of the instant invention can be recombinantly produced and supplied to any the available in vitro translation systems (such as the commercially available Wheat Germ Lysate-based PROTEINscript-PRO™, Ambion's E. coli system for coupled in vitro transcription/translation; or the rabbit reticulocyte lysate-based Retic Lysate IVT™ Kit from Ambion). Optionally, the in vitro translation system can be selectively depleted of one or more natural AARSs (by, for example, immunodepletion using immobilized antibodies against natural AARS) and/or natural amino acids so that enhanced incorporation of the analog can be achieved. Alternatively, nucleic acids encoding the re-designed AARSs may be supplied in place of recombinantly produced AARSs. The in vitro translation system is also supplied with the analogs to be incorporated into mature protein products.

Although in vitro protein synthesis usually cannot be carried out on the same scale as in vivo synthesis, in vitro methods can yield hundreds of micrograms of purified protein containing amino acid analogs. Such proteins have been produced in quantities sufficient for their characterization using circular dichroism (CD), nuclear magnetic resonance (NMR) spectrometry, and X-ray crystallography. This methodology can also be used to investigate the role of hydrophobicity, packing, side chain entropy and hydrogen bonding in determining protein stability and folding. It can also be used to probe catalytic mechanism, signal transduction and electron transfer in proteins. In addition, the properties of proteins can be modified using this methodology. For example, photocaged proteins can be generated that can be activated by photolysis, and novel chemical handles have been introduced into proteins for the site specific incorporation of optical and other spectroscopic probes.

The development of a general approach for the incorporation of amino acid analogs into proteins in vivo, directly from the growth media, would greatly enhance the power of unnatural amino acid mutagenesis. For example, the ability to synthesize large quantities of proteins containing heavy atoms would facilitate protein structure determination, and the ability to site-specifically substitute fluorophores or photocleavable groups into proteins in living cells would provide powerful tools for studying protein function in vivo. Alternatively, one might be able to enhance the properties of proteins by providing building blocks with new functional groups, such as a keto-containing amino acid.

For in vivo use, one or more AARS of the instant invention can be supplied to a host cell (prokaryotic or eukaryotic) as genetic materials, such as coding sequences on plasmids or viral vectors, which may optionally integrate into the host genome and constitutively or inducibly express the re-designed AARSs. A heterologous or endogenous protein of interest can be expressed in such a host cell, at the presence of supplied amino acid analogs. The protein products can then be purified using any art-recognized protein purification techniques, or techniques specially designed for the protein of interest.

The above described uses are merely a few possible means for generating a transcript which encodes a polypeptide. In general, any means known in the art for generating transcripts can be employed to synthesize proteins with amino acid analogs. For example, any in vitro transcription system or coupled transcription/translation systems can be used for generate a transcript of interest, which then serves as a template for protein synthesis. Alternatively, any cell, engineered cell/cell line, or functional components (lysates, membrane fractions, etc.) that is capable of expressing proteins from genetic materials can be used to generate a transcript. These means for generating a transcript will typically include such components as RNA polymerase (T7, SP6, etc.) and co-factors, nucleotides (ATP, CTP, GTP, UTP), necessary transcription factors, and appropriate buffer conditions, as well as at least one suitable DNA template, but other components may also added for optimized reaction condition. A skilled artisan would readily envision other embodiments similar to those described herein.

### EXAMPLES

The instant invention provides methods and implementing computer software for designing mutant proteins (the Target Protein or TP) that will preferentially bind one list of prespecified ligands (Active Ligands or AL) with respect to another list of ligands (The Inactive Ligands or IL). Although the examples given below use an aminoacyl-tRNA synthetases as the TP, its natural ligand as the IP, and a single modified amino acid as the AL, the methods of the invention (or “COP”) can be applied to any other protein-ligand binding system.

The example described below are for illustrative purpose only, and should not be construed to be limiting in any respect.

### Example 1

### Designing Mutant Tyrosyl-tRNA Synthetase from Methanococcus janacshii for Recognizing O-methyl-L-tyrosine

Applicants have applied the COP algorithm to design mutants of tyrosyl-tRNA synthetase from Methanococcus janacshii (M. jann-TyrRS) for selective binding of OMe-Tyr (see Scheme 1).

Since the crystal structure of mj-TyrRS is not available, Applicants predicted the three-dimensional structure for wild-type mj-TyrRS, based on a combination of the STRUCTFAST sequence alignment and structure prediction algorithm with molecular dynamics (MD) including continuum solvent forces. [To select the 5 residues to modify in their experiments, Wang et al. (Science 292, 498-500, 2001) used a sequence alignment between mj-TyrRS and Bacillus stearothermophillus tyrosyl-tRNA synthetase (bs-TyrRS).] To validate the predicted structure for mj-TyrRS, MD plus continuum solvent energies were used to demonstrate that tyrosine (Tyr) is the preferred ligand over the 19 other natural amino acids.

There are three TyrRS crystal structures in the Protein Data Bank. They are all from Bacilus stearothermophilus, with different ligands in the structures. Structure 2ts1 has no ligand, 3ts1 with Tyr-AMP bound, and 4ts1 has a Tyr in the binding site. By using the sequence of the wild-type mj-TyrRS from Genbank (accession number: Q57834), the three-dimensional structure of the main chain of mj-TyrRS was predicted with STRUCTFAST homology modeling technique. The structure of 4ts1 was used as the template in the prediction. The sequence identity between the two sequences is 32.1%. The main chain atoms of the initial predicted mf-TyrRS structure agree with the corresponding residues of 4ts1 structure to 0.64 Å in root mean square difference (rmsd) in coordinates after aligning the two structures using DALI (Holm and Sander, J. Mol. Biol. 233: 123-38, 1993). After full minimization, the main chain rmsd increases to 1.75 Å for the 139 structurally aligned residues. But the conserved five residues (Tyr32, Tyr151, Gln155, Asp158, and Gln173) in the binding site have a rmsd of 0.62 Å for all heavy atoms.

To place the Tyr ligand in the predicted structure, Applicants matched the side chain conformation of the five strictly conserved residues (Tyr32, Tyr151, Gln155, Asp158 and Gln173) in the binding site of mj-TyrRS with those conformations from the 4ts1 crystal structure. The rest of the side chains for the predicted mj-TyrRS structure were added by using side chain modeling program SCWRL version 2.7 (Bower et al., J. Mol. Biol. 267: 1268-82, 1997; Dunbrack, Proteins Suppl, 81-7, 1999) while keeping the conformations in the binding site fixed. Here Applicants used the backbone-dependent side chain rotamer library with SCWRL to optimize the side chain conformations. The potential energy of the resulting structure was then minimized using conjugate gradient method with MPSIM (supra), which allowed all the side chains to move but kept the main chain fixed. In MPSIM, the Cell Multiple Method (CMM) (Ding et al., J. Chem. Phys. 97: 4309-4315, 1992) was used to rapidly yet accurately calculate the non-bond interactions. The protein was described with the DREIDING force field (supra) with CHARMM22 (supra) charges.

For the Tyr ligand and other amino acids in the simulation, Applicants used Mulliken charges derived from the molecular orbitals in quantum mechanics (QM). The QM calculations were done at the HF level with the 6-31G** basis set. The geometry of the molecules was optimized under forces from Poisson Boltzmann continuum solvation (supra) inside of QM package Jaguar 4.0 (Schrodinger, Portland, Oreg.).

After optimizing the side chain conformations in the protein, the potential energy of the whole protein was minimized, with all atoms movable but with distance constraint on the hydrogen bonds between the phenolic OH group of the Tyr ligand and the Tyr32 and Asp158 side chains. This minimized structure was then used as a starting structure for annealing molecular dynamics (MD), where all constraints were relaxed. Each cycle of annealing MD involved heating the system from 50 to 600 K and cooling from 600 to 50 K in steps of 20 K for 0.5 ps. These annealing MD calculations included solvent forces from the Surface Generalized Born (SGB) continuum solvation method (25) with a dielectric constant of 80 for bulk solvent, 2 for protein and a solvent probe radius of 1.4 Å. The final structure was used to predict the binding of all 20 natural amino acids and to design mutant TyrRS for non-natural amino acids.

To validate the predicted mj-TyrRS structure, Applicants docked all 20 natural amino acids to the Tyr binding site of the TyrRS. The binding conformation was first minimized with protein fixed and followed by relaxing the binding site residues. The final optimization was done without any constraint on the protein. All structure optimizations were done with implicit SGB continuum solvent. To guard against misactivation noncognate amino acids, AARSs must be able to charge the correct amino acid to its corresponding tRNA. The activation step consists of the bound amino acid forming the aminoacyl adenylate complex and subsequent transferring the aminoacyl group to the 3′-end of the bound tRNA. While there are extra proofreading mechanisms to ensure the fidelity for some AARSs, many AARSs recognize their substrate with very high specificity. And TyrRS is one of them. It has been also shown that PheRS, another AARS recognizing their substrates with high specificity, has calculated bonding energies correlating well with the translational activity measured in vivo. As expected, the wild-type ligand Tyr has a much higher binding energy than any other natural amino acids. The closest binding competitors are Ala, Asn and His, but all bind at least 16 kcal/mol less favorably than Tyr. Although there are several steps involved in the selection specificity in AARSs, TyrRS has been known for a long time to be able to differentiate its cognate amino acid in the initial binding stage. Hence the binding profile validates our predicted mj-TyrRS structure.

In order to get a binding conformation for other amino acids, the Tyr ligand obtained in mj-TyrRS structure optimization was used to build 19 other amino acids. To preserve the reaction center for activating the amino acids, the contact between the zwitterions of the amino acid and the appropriate residues in the binding site was fixed.

SCWRL was used to mutate the side chain into 19 other amino acids. Each of the resulting amino acids was minimized in the binding site of the protein using conjugate gradient method. The binding energy of each amino acid is calculated as:
−ΔΔG_{binding}=ΔG₍ₚᵣₒₜₑᵢₙ₎+ΔG_{(ligand)}−ΔG_{(protein+ligand)}

where ΔG_{(protein+ligand)} is the free energy for the protein-ligand complex, while ΔG₍ₚᵣₒₜₑᵢₙ₎ and ΔG_{(ligand)} are the free energy for the protein and ligand alone, respectively. The structure optimization was always done with SGB continuum salvation. Such continuum solvation model is optimized with the potential mean force (PMF) from bulk solvent, the total energies are very close to the free energy of the system (Hendsch and Tidor, Protein Sci. 8: 1381-92, 1999). This is true especially for tight bound complexes.

In determining the low-energy rotamers for each non-natural amino acid, several rotamers over a grid were generated and the geometry was then minimized in QM using Jaguar 4.0. Only the ones with low energy were used for subsequent COP design. The Mulliken charges were also obtained from these calculations and used for these amino acids in the design.

These low-energy conformation structures are then built in Biograf using the Tyr structure as a starting structure. The atoms that are identical in the non-natural amino acids are left as where they are. This means the zwitterions of the non-natural amino acids are always in the same position as in the Tyr ligand. These structures were used as input to the COP along with the Tyr ligand and mj-TyrRS structure. COP requires that the new atoms in the non-natural amino acids be labeled as HETATM in order to cut the binding site. The cutoff distance was 6 Å for the binding site. Residues outside the cutoff distance are not included in the clash calculation, but are included in the binding energy calculation (using Equation 2 above).

Starting with the predicted mj-TyrRS structure, Applicants used the COP algorithm to design mutant mj-TyrRS for selective binding of OMe-Tyr.

Scheme 1 shows the natural amino acid Tyr, and its analog OMe-Tyr (rotamer 1), for which the mutant protein was designed.

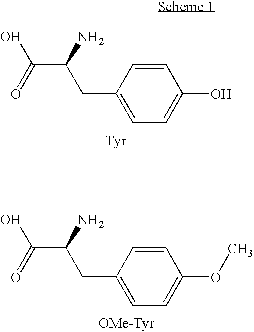

OMe-Tyr has two equally favorable rotamers, one shown in Scheme 1 (denoted 1 and the other with the —OMe group pointed down (denoted 2). Both rotamers were matched in the binding site of Tyr in wild type M. jann-TyrRS, keeping the zwitterion's end fixed in the structure. Component analysis of the contribution of each residue in the binding site to the binding of OMe-Tyr was calculated using Equation 2 (supra). The binding site was defined in this case as the entire residue for all atoms of the protein within 6 Å of any atom of the ligand. This leads to the identification of 26 binding site residues in Table 1. Since rotamer 2 had severe clashes with protein backbone residue Gly34, while rotamer 1 had none, we considered only rotamer 1 further.

TABLE 1
Interaction energies of OMe-Tyr ligand (both rotamers) and
of Tyr ligand with the predicted wild type structure of
M. jann-TyrRS. The interactions are shown for all ligands with
any atom within 6 Å of the Tyr (referred to as the binding site).

| | OMe-Tyr | OMe-Tyr | | |
|---|---|---|---|---|
| Residue | (rotamer 1) | (rotamer 2) | Tyr | Difference^{#} |
| Gln 155 | −11.15 | −10.65 | −10.66 | −0.48 |
| Met 154 | −0.93 | −0.60 | −0.60 | −0.32 |
| Ala 67 | −1.69 | −1.43 | −1.42 | −0.27 |
| Gln 109 | −0.99 | −0.91 | −0.91 | −0.08 |
| Leu 66 | −0.21 | −0.13 | −0.13 | −0.08 |
| Asn 157 | 0.13 | 0.20 | 0.20 | −0.08 |
| Val 156 | −0.24 | −0.17 | −0.17 | −0.07 |
| Phe 108 | −0.16 | −0.10 | −0.10 | −0.06 |
| Leu 65 | −1.28 | 265.94* | −1.23 | −0.05 |
| His 160 | −0.25 | −0.21 | −0.21 | −0.04 |
| Gly 105 | −0.08 | −0.03 | −0.03 | −0.04 |
| Phe 35 | −1.52 | −1.50 | −1.49 | −0.04 |
| Pro 152 | −0.32 | −0.28 | −0.28 | −0.04 |
| Ile 159 | −0.05 | −0.02 | −0.02 | −0.03 |
| Ile 33 | −0.23 | −0.21 | −0.20 | −0.03 |
| His 70 | −2.89 | −2.87 | −2.88 | −0.01 |
| Tyr 161 | −0.08 | −0.06 | −0.07 | −0.01 |
| His 177 | −0.38 | −0.39 | −0.37 | −0.01 |
| Leu 69 | 0.02 | 0.02 | 0.02 | 0.00 |
| Gly 34 | −2.04 | 302.84** | −2.06 | 0.02 |
| Gln 173 | −11.15 | −11.20 | −11.17 | 0.03 |
| Asp 68 | −0.94 | −0.98 | −0.97 | 0.03 |
| Tyr 151 | −9.44 | −9.52 | −9.66 | 0.21 |
| Glu 36 | −1.14 | −1.27 | −1.36 | 0.22 |
| Tyr 32 | −13.45 | 12745.3* | −15.69 | 2.25^{‡} |
| Asp 158 | 2450.87* | −0.80 | −15.44 | 2466.41 |

^{#}Difference between OMe-Tyr rotamer 1 and Tyr.
*Large van der Waals energy showing steric clashes of protein side chain with OMe-Tyr ligand.
**Steric clash with main chain (backbone) residue.
^{‡}Strong binding contribution to Tyr over OMe-Tyr.

The non-bond interaction energies between OMe-Tyr and residues within 6 Å of the ligand are summarized in Table 1. We find that ASP158 has a very severe clash with the OMe-Tyr ligand, and hence this residue was selected to be mutated to favor OMe-Tyr binding. In addition Tyr32 has a strong binding contribution to Tyr over OMe-Tyr and hence we targeted Tyr 32 for mutations to find a residue favorable for OMe-Tyr binding.

We considered all 20 amino acids as possible mutations for both Tyr32 and Asp158, and selected for further study all those that favor OMe-Tyr over Tyr. The results for the six most favorable mutations are shown in Table 2. This leads to the following choices to minimize clashes (stage 1):

For Tyr32: Glu, Asp, Gln, Phe, and Met.

For Asp158: Ala.

TABLE 2
Binding scores of the 6 best mutations for Tyr32 and Asp158 in M. jann-
TyrRS. Scores (in kcal/mol) are non-bond interaction energies of the
mutated residue with the OMe-Tyr or Tyr.

| | | OMe- | | | | OMe- | |
|---|---|---|---|---|---|---|---|
| Tyr32 | Tyr | Tyr | Diff | Asp158 | Tyr | Tyr | Diff |
| Glu | 0.13 | −0.28 | −0.41 | Ala | −0.41 | −0.92 | −0.51 |
| Asp | −0.14 | −0.37 | −0.23 | Gly | −0.26 | −0.08 | 0.18 |
| Gln | −0.10 | −0.28 | −0.18 | Ser | −0.52 | 2.68 | 3.20 |
| Met | −0.32 | −0.37 | −0.05 | Cys | −0.99 | 4.88 | 5.87 |
| Phe | −0.45 | −0.49 | −0.04 | Asp | −1.70 | 4.36 | 6.06 |
| Ser | −0.08 | −0.07 | 0.01 | Asn | −0.64 | 10.54 | 11.18 |

Based on these results, we selected five mutations with negative differences for Tyr32 (Glu, Asp, Gln, Met, and Phe), and one mutation with negative difference for Asp158 (Ala).

In stage 2, we used SCWRL (Side-chain placement With a Rotamer Library) to generate the side-chain configurations for the two mutated residue positions for each of the five combinations of simultaneous mutations selected from stage 1 (with 5 choices for Tyr32 and 1 for Asp158). These side-chains were optimized separately for the analog and for Tyr in the binding site. This leads to 5 full protein structures for each ligand. We then carried out energy minimization for the new side chains with all other atoms fixed. This was followed with energy minimization of the whole protein with either OMe-Tyr or Tyr bound for all five mutants. The binding energies (including salvation) for both OMe-Tyr and Tyr were then calculated for all mutants. This leads to the binding energies in the top box of Table 3 for OMe-Tyr and Tyr bound to the five mutants.

TABLE 3
Binding energies of OMe-Tyr and Tyr to the wild type M. jann-TyrRS and
mutants.

| | | | | | | Differential |
|---|---|---|---|---|---|---|
| | | | | OMe-Tyr | Tyr | (OMe-Tyr |
| Y32 | D158 | E107 | L162 | (kcal/mole) | (kcal/Mole) | v. Tyr) |
| Y | D | E | L | 12.34 | 43.83 | −56.17* |
| E | A | | | 37.11 | 37.64 | −0.54 |
| D | A | | | 43.85 | 38.24 | 5.60** |
| Q | A | | | 48.93 | 42.30 | 6.62** |
| F | A | | | 39.06 | 39.81 | −0.76 |
| M | A | | | 44.17 | 39.00 | 5.16** |
| E | A | T | P | 27.48 | 34.98 | −7.51 |
| D | A | T | P | 31.65 | 25.58 | 6.06 |
| Q | A | T | P | 27.06 | 17.72 | 9.33*** |
| F | A | T | P | 31.54 | 34.06 | −2.53 |
| M | A | T | P | 27.20 | 28.69 | −1.50 |

*Wild type M. jann-TyrRS
**Chosen designed mutant M. jann-TyrRS
***Mutant M. jann-TyrRS found experimentally (ref 16)

The first row is for the wild type, which binds Tyr well but not OMe-Try. The next five rows (boxed) consider the Mutations for Y32 and D158 identified in Table 2. The three cases denoted as ** are considered to be promising cases worth testing. The last five rows consider these same five mutations, but with the E107T and L162P mutations observed in the experiments of Wang et al., Science 292: 497-500 (see below). The case denoted as *** is the one determined experimentally.

The results for the five mutant proteins can be compared to the wild type (given in the top row of numbers in Table 3), which leads to 44 kcal/mol for Tyr but −12 kcal/mol for OMe-Tyr. All five mutants bind Tyr less strongly (38 to 42 kcal/mol) while these five mutants bind OMe-Tyr by 37 to 49 kcal/mol. Of the five mutants three favor binding of OMe-Tyr over Tyr by at least 5 kcal/mol. These are [Y32Q, D158A], [Y32M, D158A], and [Y32D, D158A], which lead to total binding energy for OMe-Tyr of 49, 44 and 44, and differential binding energies of 7, 5 and 6 kcal/mol. The other two cases ([Y32E, D158A], and [Y32F, D158A]) both lead to weak binding and favor Tyr over OMe-Tyr, hence these two mutants are ignored.

FIG. 2 shows the predicted binding site of OMe-Tyr for the best mutant, [Y32Q, D158A]. It is observed that residues Ala67, Ala158, and Leu65 form a hydrophobic pocket for the methyl group of OMe-Tyr. The amide N^{ε2} of Gln32 has close contact with the oxygen atom of the OMe group (3.79 \acute{Å}), while the O^{ε1} atom of Gln32 is stabilized by forming a weak hydrogen bound (3.58 \acute{Å}) with the main chain NH of Leu65. These H-bonds might be further stabilized by an intervening water.

The mutant [Y32M, D158A] is also a favorable candidate. However for [Y32D, D158A] the charged group of the Asp does not seem to have a favorable stabilization of the charged group which may lead to problems with folding.

Comparison to Experimental Results

Wang et al. (Science 292: 497-500) carried out a combinatorial experiment to find a mutant optimum for OMe-Tyr. Since no 3D structure was available, Wang et al. used a sequence Alignment with 4ts1. This alignment suggested that five residues, namely Y32, D158, E107, L162, and I159, are in the active site. Wang et al. then considered all 5²⁰ theoretical mutations and selected the best ones. This selection was carried out by first screening for binding of the enzyme to any amino acid, followed by screening against natural amino acids to find the mutants least able to bind Tyr and any other natural amino acids. Then of these mutants, Wang et al. considered the ones that are most selective toward OMe-Tyr. These studies eventually led to the mutant [Y32Q, D158A, E107T, L162P, I159I]. Thus the experiments of Wang et al. confirm that the [Y32Q, D158A] replacements identified by COP to be the best mutant enzyme with the desired activity.

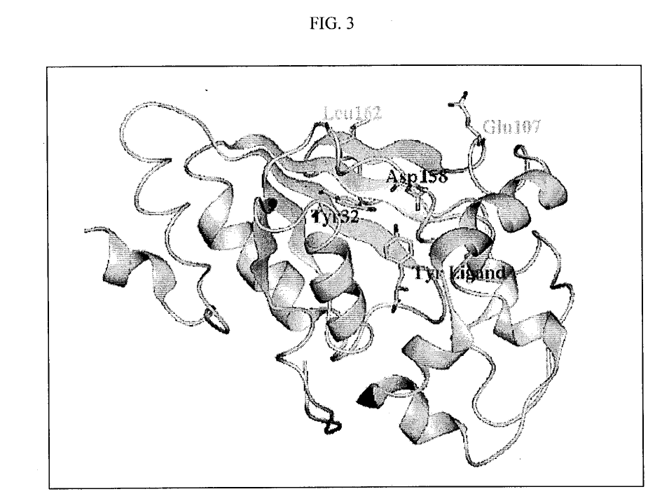

However, Wang et al. suggest that their optimum mutant also includes E107T and L162P (they found that 1159 did not change). We did not consider mutations of E107 or L162 because our predicted structure for M. jann-TyrRS places both residues far from the binding site (see FIG. 3). Thus we find that Glu107 is on the surface of the protein, 12.9 \acute{Å} away from the Tyr ligand (from the C_{α} of Glu107 to the O^{η} of the Tyr ligand), and Leu162 is 14.5 \acute{Å} away from the Tyr ligand. In Wang's alignment, Leu162 and Glu107 in M. jann-TyrRS correspond to Leu180 and Asn123 in the 4ts1 structure. Thus Leu180 is in the middle of a beta strand on the bottom of the binding site in 4ts1. The Leu-Pro mutation at this position would be expected to disrupt the secondary structure. Since Asn123 is close to the core of the protein in 4ts1, it seems unlikely that a charged Glu would fold into this structure.

Since our predicted structure puts the residues Glu107 and Leu162 well outside the binding site for M. jann-TyrRS, COP did not find these residues as mutation targets.

To understand why the combinatorial experiments would lead to a different result than the COP design, we calculated the effect of including the [E107T, L162P] mutations along with the 5 mutants from the COP analysis. The L162P mutation requires a change in the main chain conformation, and hence we carried out annealing MD to optimize the backbone structure. Therefore, the energy minimization of section 2.3 was followed by one cycle of annealing MD with SGB solvation method using MPSim. The resulting best energy annealed protein structure was used to calculate binding energies.

The calculated binding energies of both OMe-Tyr and Tyr to the mutants with the five mutations are shown in the second box of Table 3. We find that the experimental optimum mutation, [Y32Q, D158A, E107A, L162P], leads to a dramatically weak binding (18 kcal/mol) to Tyr. Since the experiments conducted negative selections against any mutant TyrRS that binds Tyr, our favored mutants, which have higher affinity to Tyr, would be screened out in this negative selection process. We also find that the experimental mutant leads to a differential preference for OMe-Tyr over Tyr of 9 kcal/mol. Although this is by far the best differential we have seen, and is even better than our designed mutant, it came with a cost of reduced net binding energy. Specifically, the net binding of OMe-Tyr to the [Y32Q, D158A, E107A, L162P] mutant is calculated to be only about 27 kcal/mol, while the net binding of Tyr to the mutant is calculated to be about 18 kcal/mol. This could explain the observation that the mutant led to an incorporation rate much slower than for the natural amino acid. Thus the calculations do seem to be consistent with the experiment of Wang et al., given how the experiment was carried out.

Since all of our predicted mutants would have been in the experimental screen, it would be interesting to reexamine the three mutants predicted by COP to determine how effective they are in vivo. It is expected that the predicted differentials of 5 to 7 kcal/mol is sufficient to obtain selectively. In addition, the total binding energies of 44 to 49 kcal/mol for OMe-Tyr suggests that these new mutants would be much more active that the experimentally identified one.

### Example 2

### Designing Mutatant mj-Tyr RNA Synthetase for Recognizing Naphthyl-Alanine

Using the same predicted mj-TyrRS, Applicants next designed the TyrRS to bind non-natural amino acid L-3-(2-naphthyl)alanine (naph-Ala). Two rotamers of the naphthyl-Ala were built from the Tyr ligand. Mulliken charges from QM calculation were assigned to naphthyl-Ala. Each of the two rotamers were matched into the binding site of Tyr, and clashes were calculated between the ligand and proteins.

The calculation showed that Q155 has a main-chain clash with rotamer 1 of naph-Ala. This eliminates rotamer 1 from further consideration. Rotamer 2 does not have any main-chain clash, therefore the following design steps are only applied to rotamer 2. Using a cutoff of 0.5 kcal/mol, two residues Y32 and D158 are selected as mutation sites. Each of the 20 amino acids is tried on the two positions one at a time. The mutated residue is minimized, and the interaction energies with naph-Ala and Tyr are evaluated. Finally a score is calculated for each mutation, as described in the section above, using 95% of the non-bond interaction with ligands plus 5% of the constraint energy for the mutated residue with neighboring residues within the protein. These calculations identified 12 choices for Tyr32 (I, Q, V, N, M, T, L, P, C, A, G, S), and two choices for Asp58 (G and A). A cutoff value of 0 is used to choose mutations that favor naph-Ala over Tyr. In the next step, 12×2=24 mutants are generated. The mutants are first minimized with only the mutation residues movable and the rest of the protein and ligand fixed, followed by a full optimization. Equation 2 is then used to score each mutants for binding with naph-Ala and Tyr. Two possible competitors from natural amino acids, Trp and Phe, are also scored for binding to the mutants designed. Normally, this procedure is only performed for mutants with high affinity to the designed analog, however, every mutants are evaluated with competitors here because there are only 24 mutants designed.

Applicants found that all these COP designed mutants have good binding energies to naph-Tyr and better binding energy than any of the competitors (results not shown). Among these mutants, there are seven of them having binding energies at least 5 kcal/mol better than any of the competitors. And the stability checks performed on them all indicate they can fold into the native state without any problem.

Further, Applicants compare these mutants with the experimental mutant selected from a library of 5²⁰ mutants with five positions each replaced by one of the 20 natural amino acids (Wang et al., J. Am. Chem. Soc. 124: 1836-1837, 2002). These five positions are Y32, D158, I159, L162, and A167. And the experimental mutant is Y32L-D158P-I159A-L162Q-A167V. Compared with COP designed mutants, the first two mutation sites are the same as what COP found. However, the other three residues I159, L162 and A167 are not in contact with the analog naph-Ala, thus COP did not identify them as mutation residues. The mutation Y32L also appears in two designed mutants with good binding affinity toward naph-Ala. On the other hand, P was not a choice for D158 in COP design. The reason is that it requires main chain conformational change in the mutation D158P. The phi/psi angles for D158 in mj-TyrRS is −58°/113°, while for P it should be either −57°/−38° or −63°/139°. We performed a simulation by making a mutant with all the mutations found in experiment. Due to the main chain conformational change in the D158P mutation, an annealing dynamics was carried out on the mutant to allow the back bone of the mutant to adjust to an optimal position. Simulation showed the phi/psi angles were −53°/125° in the optimized mutant. The mutation V159A facilitated the main chain conformational change by allowing the backbone move further away from the ligand.

Applicants also calculated the binding energies of naph-Ala and its competitors (Tyr, Phe, Trp). As a comparison, the wild-type mj-TyrRS and a few COP designed mutants are also listed. The experimentally selected mutant has the worst binding energy to Tyr, which is also the best binding natural amino acid among the competitors to naph-Ala. This is consistent with the experimental procedure, in which several rounds of negative selection against natural amino acids were performed. Our simulation shows that that procedure selected the mutant with the worst binding affinity to natural amino acids, however, neither the affinity to naph-Ala nor the differential binding affinity to naph-Ala was optimized in the experiment. Such selected mutants usually only have moderate binding affinity to the analogs intended to design for. In this case, four of COP designed mutants have better binding affinity to naph-Ala with at least the same differential binding affinity to naph-Ala. For more details, see Ph.D. thesis of DeQiang Zhang (http://etd.caltech.edu/etd/available/etd-12182002-190040/, incorporated by reference).

### Example 3

### Designing Mutatant mj-Tyr RNA Synthetase for Recognizing p-keto-Tyr

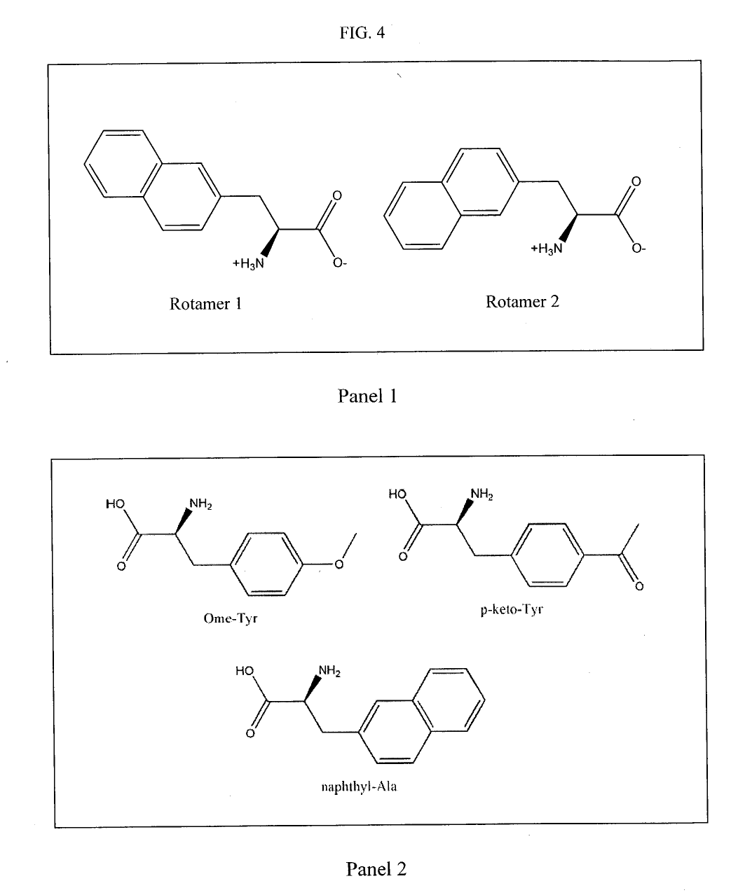

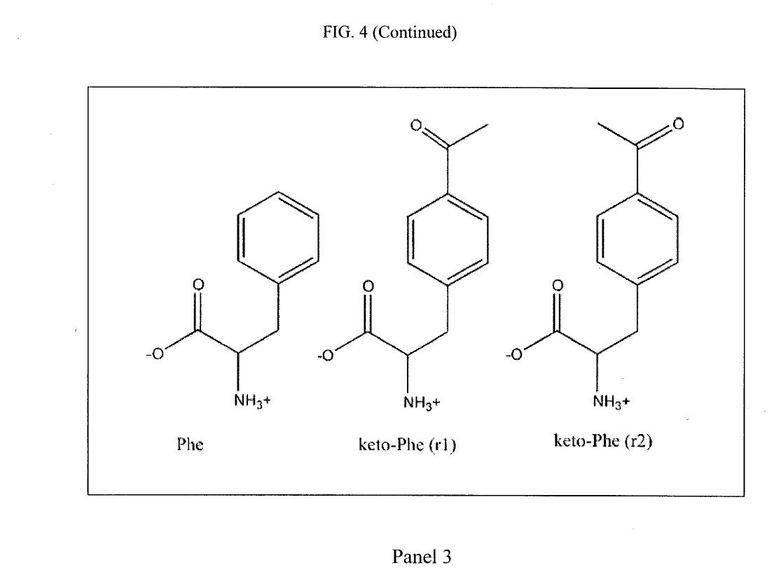

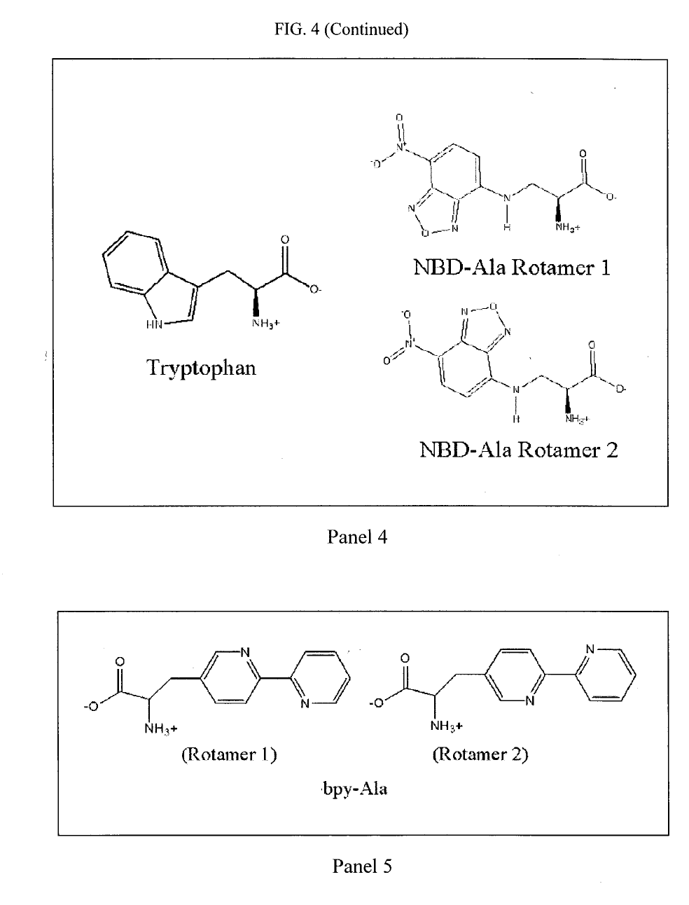

Using the same method above, two low energy rotamers of keto-Tyr (see panel 2 of FIG. 4) were built in Biograf. The carbonyl group is conjugate with the aromatic ring in these two rotamers. Again Mulliken charges from quantum mechanics were used for ligands. Both rotamers of keto-Tyr were matched into the binding site of Tyr in mj-TyrRS. Clashes were calculated using COP. The interactions between keto-Tyr, Tyr and residues in the binding site of mj-TyrRS were calculated as above. Based on the calculation, rotamer 2 has main chain clash with Q155, and rotamer 1 has no main chain clash, therefore rotamer 2 was not used in further steps and only rotamer I was further considered.

There are two residues having severe clash with keto-Tyr, and they are Y32 and D158. A third residue, Q155, has a less favorable interaction with keto-Tyr than Tyr. However, this residue is not considered as a clash residue, because the interaction with keto-Tyr is negative. Further Q155 is an essential residue anchoring the amino acid binding by forming a hydrogen bond with the zwitterions of the amino acid ligand. Hence, Q155 was not included in the mutation list. The hydrogen bond design procedure indicates no optimal hydrogen bond residue can be found for the carbonyl group.

Next step is to try all 20 amino acids in position Y32 and D158 one at a time. The mutated residue is minimized with everything else fixed first, followed by calculating the interaction energy of the mutation with the ligands and the rest of the protein. A preferential score for each amino acid is then calculated as 95% of the differential interaction energy with keto-Tyr and with Tyr, plus 5% of the constraint energy of the mutated residue with its neighbor residues in protein.

These calculations identified 11 choices (M, Q, N, I, V, L, T, P, C, A, G) for Y32, and one choice (G) for D158, using a cutoff of 0 kcal/mol in score. However, it is always safe to add the next choice to the list when there is only one choice. Therefore (G, A) was chosen for D158. The combined mutation generates 11×2=22 mutants. The binding energies of each mutant for keto-Tyr and its competitors are calculated using Equation 2.

There are only three mutants that have better binding affinity to keto-Tyr than its competitors from natural amino acids and have no problem folding into the native fold. Phe seems to be the main competitor in the design here, which makes sense because both polar residues recognizing Tyr over Phe are being mutated to less polar residues. The best mutant is Y32G-D158G with a 1.37 kcal/mol binding energy better than Phe, and the next two mutants Y32M-D158G and Y32A-D158G both have less than 1 kcal/mol binding energy better than Phe. For more details, see Ph.D. thesis of DeQiang Zhang (http://etd.caltech.edu/etd/available/etd-12182002-190040/, incorporated by reference).

### Example 4

### Designing Mutatant Phe-tRNA Synthetase for Recognizing p-keto-Phe

The experiment of keto-Phe incorporation was done in E. coli. However, there is no crystal structure available for E. coli phenylAlanyl-tRNA synthetase (PheRS) in the PDB. Instead PheRS from Thermus thermophilus has been crystallized and solved under different conditions previously (18-21). Because the homology between PheRSs from E. coli and T. Thermophilus is very high (46.2% identical residues, with only a few deletions), we used PheRS from T. thermophilus as the modeling system. The structure of PheRS complexed with Phe (PDB ID: 1B70 resolution 2.7 Å) was downloaded from the Protein Data Bank, and hydrogens were added using Biograf (Accelrys, San Diego, Calif.). The structure was minimized with conjugate gradient method to an rms force of 0.1 kcal/mol/Å or maximum of 5000 steps. Dreiding force field (supra) was used for energy expression. The protein was described using CHARMM22 (supra) charges, while charges for the ligands were Mulliken charges derived from molecular orbitals in quantum mechanics using Jaguar 4.5 (Schrödinger, Portland, Oreg.). The minimized structure was used in the design.

The keto-Phe analog was built from the Phe ligand in the minimized structure. There are two rotamers with equally favorable energies (see panel 3 of FIG. 4). For the opportunity part, Applicants implemented a rotamer library based procedure to build potential hydrogen bonds between the protein and the new ligand. The library was based on the protein side chain rotamer library used by SCAP (supra). First the new analog ligand is compared with the wild-type ligand to see if there is extra polar atom for the analog ligand. If no such atom found for the analog, no hydrogen bond building is necessary. Depending on the polarity of the polar atom in the analog, all residues within 6 Å of the polar atom are considered to build a new hydrogen bond donor/acceptor residue. Each rotamer from the side chain rotamer library is tried to see if there is a hydrogen bond can be formed between that residue and the polar atom in the analog. Rotamers that clash with the backbone of the protein and the analog will be eliminated. Once a hydrogen bond forming residue is found in this way, other residues that have side chain clash with it will be mutated to eliminate the clash. These mutations will be added to the list of mutations from clash identification and van der Waals interaction opportunity optimization. This part can be done by visualization, but there is a level of uncertainty as to which residue is a good target. For mutations from opportunity, the original choice of residue type is preferably always included, because these mutations are not absolutely necessary. In addition, in the scoring method, Analytical Volume Generalized Born (AVGB) salvation may be used to better account for the solvation effect for protein/ligands.

Finally, if certain designed mutants have been proved to be unable to fold correctly due to unfavorable interactions caused by the mutation (such as when we put a charged residue in the protein core), an individual residue interaction energy test may be used to alleviate the problem. This energy test was based on the statistics of the interaction energy of residues in AARSs. The interaction energy of each residue in the binding site with its neighbors is calculated. A score s(n) for residue n is calculated by
s(n)=[E(n)−E(AA)]/σ(AA)

where E(n) is the interaction energy of residue n with its neighboring residues, E(AA) and σ(AA) are the interaction energy and the standard deviation of residue type AA from statistics. Table X above shows the values used in the COP procedure. A score higher than 2 usually indicates a high possibility that the designed mutant has folding problem.

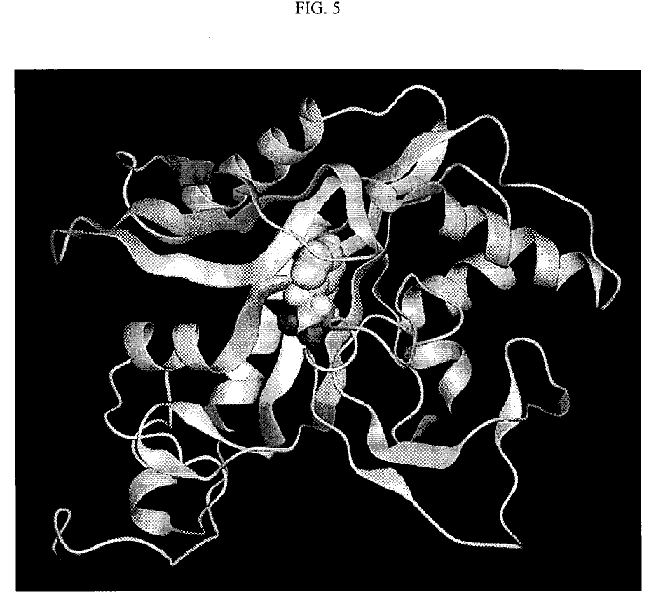

The PheRS/Phe complex was minimized as described above. The rmsd for the complex between and after minimization was 0.20 Å. FIG. 5 is the minimized PheRS in ribbon representation with Phe shown as ball model. The two rotamers of keto-Phe were built from the Phe ligand in the minimized PheRS/Phe complex structure. Each rotamers of keto-Phe was matched into the binding site of Phe in PheRS, and clashes were calculated for each rotamer. It is shown that rotamer 1 of keto-Phe clashes with the protein backbone with G284 and A283, while rotamer 2 does not clash with any backbone atoms. Therefore, only rotamer 2 was used in the following steps of design. Two residues V261 and A314 were identified as mutation target residues. Each of them was mutated into all 20 amino acids using scwr1. A backbone-dependent rotamer library was used to place the side chain conformation with the lowest constraint energy in the mutation site. Using a cutoff of 0 kcal/mol, only one choice for both V261 and A314 were selected, and both were Gly. There is a polar oxygen atom in the keto group. However, the hydrogen bond design algorithm in COP did not find optimal hydrogen bonds for the keto group. Thus COP designed a V261 G-A314G mutant only.

Previously we did the hydrogen bond design part by visualization and decided to build a hydrogen bond donor residue on V286. We also tried to make room for V286 mutation by make L222 to smaller residues. Both rotamers of keto-Phe and some competitors (Phe and Tyr in this case) were used as binding ligands. As a test, the binding energy to the wild-type PheRS and an A314G mutant were also calculated. The A314G mutant has been previously shown to be able to bind p-Br—Phe.

V261G-A314G-V286R was a mutant we designed previously using visualization as a procedure to build hydrogen bond between the protein and the analog. Using the new hydrogen bond builder with a side chain rotamer library, COP did not choose any residue to build hydrogen bond donors. Also using the new scoring method, this mutant now shows a less favorable binding energy than Tyr, thus it will also be rejected. The stability check also failed to give a stable protein fold. The V261G-A314G mutant shows a good differential binding energy between keto-Phe and its competitors, Phe and Tyr in this case. It favors keto-Phe binding by 7.45 kcal/mol better than Tyr, the closest competitor. FIG. 3 shows the binding site of keto-Phe in the designed V261G-A314G mutant.

There is no specific polar interaction with the carbonyl group of the side chain of keto-Phe from the protein, i.e., no hydrogen bond is formed. The two mutations V261 G and A314G enable the binding of keto-Phe by making the binding site larger to accommodate the extra acetyl group in the side chain of keto-Phe. Other interactions remain the same as seen in the wild-type Phe-PheRS complex. Resides 258-261, 282-284, and 314-316 form the binding pocket for the side chain of keto-Phe. On the zwitterions part, E220, S180 and Q218 form hydrogen bonds with the N-terminus, and W149, H178 and R204 form hydrogen bonds with the C-terminus. These interactions anchors the amino acid ligand specifically for activation with ATP to form aminoacyl adenylate complex.

In summary, Applicants have applied the COP protein design tool to design mutant PheRS for the in vivo incorporation of p-keto-Phe. A mutant V261 G-A314G was designed, and showed good binding affinity to keto-Phe and good differential binding to keto-Phe than its competitors from the natural amino acids the mutant has been experimentally tested to be able to recognize the target analog p-keto-phenylAlanine (keto-Phe).

### Example 5

### Designing Mutatant Trp-tRNA Synthetase for Recognizing Non-Natural Amino Acid Analogs

Many amino acid analogs with interesting properties are significantly larger than tyrosine and phenylAlanine, and thus it might be beneficial to used tryptophanyl-tRNA synthetase as a template to design mutant synthetases, in order to alleviate potential main chain clashes. This is partly because the binding site of Trp is significantly larger than Tyr and Phe, as a result the chance of backbone clash with the protein is smaller. Here we have used COP to design mutant TrpRS for recognizing 2-amino-3-(7-nitro-benzo [1,2,5]oxadiazol-4-ylamino)-propionic acid (NBD-Ala), and 2-amino-3-[2,2′]bipyridinyl-5-yl-propionic acid (bpy-Ala). Some good mutants that have good differential binding energy to these analogs over natural amino acids have been designed.

The crystal structure of TrpRS from B. stearothermophilus with Trp bound in the binding site (PDB: 1I6M, resolution: 1.72 Å) was downloaded from the Protein Data Bank. Hydrogens were added to the structure using Biograf (Accelrys, San Diego, Calif.), followed by annealing on the hydrogens to optimize the hydrogen bond network. The heavy atoms were fixed during the annealing. The structure was subject to further optimization using conjugate gradient minimization with all atoms movable for 2000 steps and with a convergence criterion of rms force reaching less than 0.1 kcal/mol/Å. The simulation program MPSim (supra) was used along DREIDING force field (supra). Surface Generalized Born (SGB) continuum solvation (supra) was included in the optimization to account for the solvation effect. The protein was described with CHARMM22 charges (supra), while the ligand Trp was with Mulliken charges derived from molecular orbitals in quantum mechanics (QM). The QM calculation was carried out at HF level using 6-31G** basis set in Jaguar 4.5 (Schrödinger, Portland, Oreg.). The geometry optimization was performed with forces calculated from Poisson-Boltzmann continuum dielectric solvent (supra). The same Mulliken charges were used for the two analogs and other competing natural amino acids. The rms deviation between the optimized complex structure and the original crystal structure was 0.32 Å. And the protein TrpRS and ligand Trp were split from the complex for use in the mutant TrpRS design.

Design for NBD-Ala

Several low energy rotamers of the analog is built from the wild-type amino acid. The zwitterions part of the analog is the same as that of the ligand. Then each of the rotamers of the analog is put into the binding site of the protein, and Equation 1 is used to calculate the nonbond interaction between the analog and residue k in the binding site. The same is done for the wild-type amino acid ligand, and the difference of the interaction energy with the analog and the wild-type amino acid is calculated for each residue. Those residues that have an unfavorable binding contribution to the analog will show positive differential interaction energies. These residues either clash with the analog or do not make enough interactions with the analog, thus they represent the opportunity to improve the binding to the analog. These residues will be mutated into all 20 natural amino acids to find choices that can either relieve the clash or improve the interaction with the analog. A score is calculated by considering the differential interaction energy of the mutated residue with the analog and the wild-type amino acid, the constraint energy of the mutated residue with the rest of the protein. Mutations with positive scores are selected for use in making mutants in the combined stage. The mutant proteins are then generated by combining the choices for each mutation site, and optimized by minimization. The binding energy of the analog to the mutant is calculated with Equation 2. The binding energies of competitors from natural amino acids are also calculated, and the differential binding energy between the analog and the best competitor is used as a criterion to select mutants. These mutants will be checked by stability of each mutated residues to make sure that they make enough interactions with the rest of the protein so that the fold is stable.

Two rotamers of NBD-Ala with low energy were built in Biograf using the same coordinates for zwittenions as in wild-type Trp ligand. These two rotamers along with Trp are shown in panel 4 of FIG. 4. There are other rotamers for NBD-Ala, however, they have less overlap with the binding site of Trp and almost certainly they will clash with the protein backbone in the design using COP. Hence, only the two rotamers shown here were used. The two rotamers of NBD-Ala were placed into the binding site of TrpRS, and the interaction energies of each residue with NBD-Ala and Trp ligand were calculated using Equation 1. The difference of the interaction between NBD-Ala and Trp was then calculated. The binding site is defined as within 6 Å of the side chain of NBD-Ala. Five residues (V141, V143, D132, M129 and F5) were found to have less favorable interactions with rotamer 1 of NBD-Ala compared to Trp. The same five residues had less favorable interactions with rotamer 2 of NBD-Ala. Here a cutoff value of 1 kcal/mol was used to select residues to mutate. There were no main chain clash between either rotamer of NBD-Ala and the protein. Therefore, both rotamers were used in further design steps. Each residue was mutated into 20 natural amino acids one by one, and the interaction energies between the mutated residue and ligands (NBD-Ala and Trp) again were calculated using Equation 1. A score was assigned to each mutation by combining 95% of the differential interaction energy with the mutated residue between NBD-Ala and Trp, plus 5% of the constraint energy of the mutated residue with the rest of the protein. A score cutoff of 0 kcal/mol was used to select good mutations favoring NBD-Ala binding over Trp. Note that the interaction energy between the mutated residue and NBD-Ala had to be favorable for the mutation to be chosen. Because the interaction energy of these residues with NBD-Ala were all positive except one (F5), the original choice of each residue was also included in the mutation lists. The reason for doing this is that some mutants might need only three or less mutations to achieve optimal selection.

For rotamer 1, G, A, Q are chosen for V141; A, T, N, G, C are chosen for V143; I, M, N, G, S, A, H, C, T, P, V are chosen for D132; M, L, I are chosen for M129; F, W, N are chosen for F5. For rotamer 2, A, G, Q are chosen for V141; T, N, C, A, S, G are chosen for V143; Q, W, L, I, M, G are chosen for D132; M, L, I, N, S are chosen for M129; Y, F are chosen for F5.

These mutations were combined to make mutant TrpRS. A total number of 3×5×11×3×3=1485 mutants were generated for rotamer 1 of NBD-Ala, and for rotamer 2 the number is 3×6×6×5×2=1080. These mutants were each scored by its binding energy to NBD-Ala calculated using Equation 2. A cutoff binding energy of 25 kcal/mol was used to select mutants with good binding energy to NBD-Ala. These selected mutants were further scored with binding energy to Trp, Tyr and Phe, because they were thought to be the main competitors from natural amino acids due to their similarity to NBD-Ala. The mutants were ranked by the differential binding energy between NBD-Ala and the best competitor among Trp, Tyr and Phe. Finally a stability check for each mutation was performed for each mutant. Those mutants with mutations making unfavorable protein-protein interactions were discarded due to their possible problem with folding.

The top mutants designed by COP for rotamer 1 of NBD-Ala are: (1) [V141G, V143T, D132M, M129M, F5F]; (2) [V141G, V143C, D132M, M129M, F5F]; (3) [V141A, V143T, D132M, M129M, F5F]; (4) [V141A, V143G, D132M, M129M, F5F]; (5) [V141G, V143C, D1321, M1291, F5F]; (6) [V141G, V143T, D132I, M129I, F5F]; (7) [V141A, V143A, D132M, M129M, F5F]; (8) [V141A, V143G, D132I, M129I, F5F]; (9) [V141A, V143T, D132I, M129I, F5F]; (10) [V141A, V143C, D132I, M129T, F5F]; (11) [V141A, V143A, D132I, M129I, F5F].

The top mutants designed by COP for rotamer 2 of NBD-Ala are: (1) [V141G, V143T, D132I, M129L, F5F]; (2) [V141G, V143T, D132I, M129N, F5F]; (3) [V141G, V143T, D132L, M129S, F5F]; (4) [V141A, V143T, D132L, M129S, F5F]; (5) [V141G, V143T, D132M, M129I, F5F]; (6) [V141A, V143T, D132M, M129I, F5F]; (7) [V141G, V143T, D132M, M129L, F5F]; (8) [V141G, V143C, D132L, M129N, F5F]; (9) [V141G, V143T, D132I, M129M, F5F]; (10) [V141G, V143S, D132I, M129I, F5F]; (11) [V141A, V143T, D132M, M129L, F5F]; (12) [V141G, V143C, D132I, M129I, F5F]; (13) [V141A, V143S, D132I, M129I, F5F].

Among the mutants designed based on rotamer 1 of NBD-Ala, F5 remained F for all of them. Interestingly V141 and M129 were always mutated to the same residue. The V141 mutation, which had severe clash with NBD-Ala before mutation, can be either G or A. This mutation is presumably for relieving the clash between the nitro group of NBD-Ala and the protein. M129 seemed to be less critical as it could be either M or I. Both seem to have the same size. The V143T mutation formed a hydrogen bond with the nitro group. D132 recognizes the side chain NH of the Trp ligand in wild-type TrpRS, and mutation D132I blocks the normal binding of Trp. As a result, Trp is hardly a competitor in the competitive binding.

Thirteen mutants were designed based on rotamer 2 of NBD-Ala. Similar to the case of rotamer 1, mutation V141 to G or A allows the nitro group to go in further in the binding site, and V143T forms a hydrogen bond with the five-member ring of the NBD. The choice for D132 was among M, L and I. Again, this mutation blocks the binding of Trp, and Trp indeed binds poorly to these mutants. The position M129 seems to be less discriminative, and can be any of L, M, S, I or N. F5 did not change in these mutants.

Design for bpy-Ala

The same design protocol was applied for bpy-Ala. Quantum mechanics calculation using Jaguar showed that the trans-conformation was 8.39 kcal/mol lower in energy than the cis-conformation. Bpy-Ala is a good binding agent to transitional metal ions. In free solution it is usually in trans-conformation, and upon binding it switches to cis-conformation to form coordinated binding from both nitrogen atoms. Hence only the trans-conformation was used in preparing the rotamers. Two low energy rotamers in trans-conformation were built in Biograf These two rotamers were shown in panel 5 of FIG. 4. To ensure that the binding mode was the same as for Trp, the coordinates of the zwitterions were obtained from Trp.

When these rotamers were simply placed in the binding site of TrpRS, they had very bad clash with the backbone of TrpRS. The reason was that these rotamers were not aligned optimally with the binding site for Trp. However, the alignment is significantly better after optimization. In order to optimize the orientation of the side chain of bpy-Ala, the following procedure was adopted to get the conformation with lowest backbone clash energy. First a mutant TrpRS with all Gly was generated, i.e., all the side chains of the protein were removed. A grid of conformations for bpy-Ala was then generated by changing the χ1 and χ2 angles (as usual, χ1 was defined as the dihedral angle of N—Cα-Cβ-Cγ, and χ2 was defined as the dihedral angle of Cα-Cβ-Cγ-Cδ). Finally the bpy-Ala analog was put into the binding site of the all Gly TrpRS, and the energy of the complex was calculated after 10 steps of steepest descents minimization of the analog with the protein fixed. The energy was plotted in a two-dimensional energy surface plot. The minimum in the energy surface plot for rotamer 1 of bpy-Ala had χ1=−78.5o and χ2=150o, and for rotamer 2 the minimum was at χ1=−76o and χ2=160o. The conformation of the two rotamers was adjusted to these values. These two rotamers were then placed in the binding site of the wild-type TrpRS, and the binding contribution from each residue in the binding site was calculated using Equation 1. The same was done for the Trp ligand, and the difference was calculated between bpy-Ala (both rotamers) and Trp for each residue.

These calculations indicate that both rotamers still had clash with the backbone of the protein. However, the backbone clash was not very severe, and usually several steps of minimization could relieve these clashes significantly. Rotamer 1 had 8 residues that need to be mutated if the cutoff value of 0.5 kcal/mol is used. Therefore, rotamer 2 was chosen for design first since it had less main chain clash and only 5 mutations to do. The 5 mutation residues were F5, D132, I133, V40, and M129. A cutoff of 0.5 kcal/mol was used. Applicants then tried to mutate each of the five residues to all 20 natural amino acids one by one. The mutated residue conformation was chosen from a rotamer library with the lowest energy rotamer being selected. A score was calculated as 95% of the differential interactiop energy of the mutated residue with bpy-Ala and Trp, plus 5% of the constraint energy between the mutated residue and the rest of the protein. Mutations with positive score were chosen. For F5, G was chosen; for D132, G and A were chosen; for I133, T, S, M, A, G, C were chosen; for V40, A and G were chosen; for M129, M, L, I, F, H, C, V were chosen.

The mutants were generated by combining the mutations from each residue, and a total number of 1×2×6×2×7=168 mutants were generated. Each mutant was scored by the binding energy to bpy-Ala using Equation 2. A cutoff of 25 kcal/mol was used to select good binding mutants to calculate binding energies to competitors from natural amino acids. Here we assumed Trp, Tyr and Phe as competitors because of their similar sizes. The difference of the binding energy between bpy-Ala and the competitor with the best binding energy was calculated and used to select best mutants. Finally a stability check for each mutation was performed for each mutant. Those mutants with mutations making unfavorable protein-protein interactions were discarded due to their possible problem with folding.

In all, 14 of those mutants with binding energy to bpy-Ala at least 5 kcal/mol better than any of the competitors are chosen. The top seven are: (1) [F5G, D132A, I133T, V40A, M129C]; (2) [F5G, D132A, I133A, V40A, M129C]; (3) [F5G, D132A, I133S, V40A, M129C]; (4) [F5G, D132G, I133T, V40A, M129C]; (5) [F5G, D132G, I133A, V40A, M129C]; (6) [F5G, D132G, I133S, V40A, M129C]; (7) [F5G, D132A, I133M, V40A, M129C].

Among the seven mutants designed by COP, three residues have the same mutation in all of them. These mutations are F5G, V40A and M129C. D132 is mutated to either G or A. I133, which has slight clash with bpy-Ala in main chain, can be mutated into A, S, T, or M. In mutant F5G-D132A-I133T-V40A-M129C, it can be seen that the extra six-member ring takes the space opened by mutation F5G. D132A also opens some space for the extra six-member ring in bpy-Ala. Other mutations contribute to the binding of bpy-Ala by shaping the binding site according to the orientation assumed by bpy-Ala upop its binding. V40A makes the orientation of the Cβ-Cγ bond possible. I133T seems to form a weak hydrogen bond with the nitrogen atom in the second six-member ring in bpy-Ala. The distance between the nitrogen atom and the Oγ1 in T133 is 3.2 \acute{Å}. For more details of these two designs, see Ph.D. thesis of DeQiang Zhang (http://etd.caltech.edu/etd/available/etd-12182002-190040/, incorporated by reference).

### Example 6

### Designing Mutatant Phe-tRNA Synthetase for Recognizing Naphthyl-Ala

The incorporation of non-natural amino acids in vivo is mostly determined by the recognition of the non-natural amino acids by aminoacyl-tRNA synthetases. Manipulation of the activity of aminoacyl-tRNA synthetases provides a way to significantly increase the number of non-natural amino acids that can be incorporated in vivo. Applicants report here a mutant phenylAlanyl-tRNA synthetase designed by our protein design method COP to recognize naphthyl-Alanine, an analog of phenylAlanine with significant difference in size. The mutant has three mutations (V261G, F2581, and A314G), and computationally shows both a good binding affinity to naphthyl-Alanine (31.25 kcal/mol) and a good differentiation toward tryptophan (6.08 kcal/mol), the closest competitor from natural amino acids. This mutant was selected from 54 mutants Applicants tried using the COP method.

Introduction

The aminoacyl-tRNA synthetases (AARSs) catalyze the esterification of amino acids to their cognate tRNAs. The accuracy of the reaction is essential due to its nature of protein biosynthesis fidelity. On the other hand, protein biosynthesis is a great tool to make biomaterials with precise control over sequence, structure and function. In natural the monomer pool is limited to the 20 natural amino acids. It has been shown that the monomer pool of amino acids can be increased by incorporating some non-natural amino acids using the wild-type AARS apparatus. These bioderived polymers are controlled by a genetic sequence, but have novel yet well controlled architectures. However, the number of non-natural amino acids incorporated into protein using wild-type AARSs is small, and the functionalities carried by these non-natural amino acids are very limited. Typically these non-natural amino acids are analogs of natural amino acids with little difference in the side chain. There are many non-natural amino acids that have desired chemical or physical properties cannot be incorporated this way. The most important reason is that these amino acid analogs are very different from their natural amino acid counterpart. Therefore, they are rejected by the AARSs in the esterification to tRNAs.

In order to overcome the limitations, it is desirable to manipulate the activity of AARSs to make them recognize non-natural amino acids. Another reason for doing AARS activity manipulation is the promise of expanding the genetic codes by developing novel tRNA:AARS pairs orthogonal to existing such pairs in cells. It is typically done by evolving the suppressor tRNA with nonsense codon to pair with a cross-species mutant AARS, which recognizes a non-natural amino acid instead of one of the natural amino acids. The design of mutant AARS has been the bottleneck in this process due to the lack of an effective mutant screening method. The current technique screens a library of AARS mutants, in which several positions are replaced by all 20 amino acids. Five such positions will generate 5²⁰ mutants. There has been some success, but it is very time-consuming and cumbersome.

We have previously developed a Clash Opportunity Progressive (COP) protein design tool for the purpose of mutant AARS design, and used it to design AARSs to recognize non-natural amino acids. Here COP has been applied to design a mutant phenylAlanyl-tRNA synthetase (PheRS) to recognize naphthyl-Alanine (naph-Ala), which is an analog of Phe with significant increase in size on its side chain. A total of 54 mutants were generated and only one was selected from them. The designed mutant has three mutations (V261G, F2581, and A314G), and computationally shows both a good binding affinity to naph-Ala (31.25 kcal/mol) and a good differentiation toward Trp (6.08 kcal/mol), the closest competitor from natural amino acids.

Methods

The first step in COP is to prepare the structures of protein and ligands (both the natural amino acid Phe and non-natural amino acid naph-Ala here). The crystal structure of PheRS from Thermus thermbphilus (PDB: 1B70, resolution 2.7 Å) was downloaded from Protein Databank. Hydrogen was added using Biograf (Accelrys, San Diego, Calif.) and the potential energy of the structure was subsequently minimized with DREIDING force field (see Mayo et al., J. Phys. Chem. 1990, 94:8897) in MPSim (supra). The termination criterion was 0.1 kcal/mol/Å in RMS force or maximum 2000 steps, and the minimization method was conjugate gradient. CHARMM22 charges (Brooks et al. J Comput Chem 4:187-217, 1983; MacKerell et al., J. Phys. Chem. 102: 3586-3616, 1998) were used for protein and Mulliken charges were used for ligands (naph-Ala and all natural amino acids used here). Mulliken charges were derived from quantum mechanics using Jaguar (Schrodinger, Portland, Oreg.). The quantum mechanics calculation was done at HF level with 6-31G** basis set, and Poisson-Boltzmann dielectric continuum solvent was included to simulate solvation in water (supra).

Two rotamers of naph-Ala were built from the ligand Phe in the minimized protein ligand complex structure. Panel 1 of FIG. 4 shows the structure of the two rotamers.

Each of the two rotamers of naph-Ala was matched into the binding site of PheRS, and the interaction energy of residue k (Eₖ) with naph-Ala was calculated using Equation 1 (Eq. 1):

$${E}_{k} = {\sum }_{i , j} ( \frac{{q}_{i} {q}_{j}}{4 πɛ {r}_{ij}} + {D}_{e} ( {( \frac{{r}_{m}}{{r}_{ij}} )}^{12} - 2 {( \frac{{r}_{m}}{{r}_{ij}} )}^{6} ) + {D}_{HB} ( 5 {( \frac{{r}_{HB}}{{r}_{ij}} )}^{12} - 6 {( \frac{{r}_{HB}}{{r}_{ij}} )}^{10} ) {cos}^{4} θ ) \qquad ( Eq . 1 )$$

where i and j sum over all atoms in the ligand and protein residue k, of interest, qᵢ and qⱼ are partial charges on atoms i and j, respectively. rᵢⱼ is the distance between atoms i and j, and rₘ and Dₑ are van der Waals distance and well depth of atoms i and j, r_{HB} and D_{HB} are hydrogen bond distance and well depth, respectively. θ is the hydrogen bond angle between atoms i, j and their bridging hydrogen atom. Please note that the hydrogen bond term is only evaluated for hydrogen bond pair atoms. When there is no bridging hydrogen atom for i and j, the hydrogen bond term is turned off.

The same is done for the wild-type amino acid ligand, and the difference of the interaction energy with the analog and the wild-type amino acid is calculated for each residue. Those residues that have an unfavorable binding contribution to the analog will show positive differential interaction energies. These residues either clash with the analog or do not make enough interactions with the analog, thus they represent the opportunity to improve the binding to the analog. These residues will be mutated into all 20 natural amino acids to find choices that can either relieve the clash or improve the interaction with the analog. A score is calculated by considering the differential interaction energy of the mutated residue with the analog and the wild-type amino acid, the constraint energy of the mutated residue with the rest of the protein. Mutations with positive scores are selected for use in making mutants in the combined stage.

The mutant proteins are then generated by combining the choices for each mutation site, and optimized by minimization. The binding energy of the analog to the mutant is calculated as Equation 2 (Eq. 2):
−ΔΔG_{binding}=ΔG(protein)+ΔG(ligand)−ΔG(protein+ligand) (Eq. 2)

where ΔG₍ₚᵣₒₜₑᵢₙ₎, ΔG_{(ligand)} and ΔG_{(protein+ligand)} are the free energies of the protein, the ligand, and the protein ligand complex, respectively. Analytical Volume Generalized Born (AVGB) continuum solvation was included in all calculations to account for the solvation effect for protein/ligands.

The binding energies of competitors from natural amino acids are also calculated, and the differential binding energy between the analog and the best competitor is used as a criterion to select mutants. These mutants will be checked by stability of each mutated residues to make sure that they make enough interactions with the rest of the protein so that the fold is stable.

Results and Discussion

The PheRS/Phe complex was minimized as described in the methods section. The RMSD for the complex between and after minimization was 0.20 Å. This showed that our force field was compatible with the parameters used in the original structure determination. FIG. 5 is the minimized:PheRS in ribbon representation with Phe shown as ball model.

Both rotamers of naph-Ala shown in Panel 1 of FIG. 4 were matched into the binding site of PheRS, and clashes were calculated using Equation 1. This was also done for the wild-type ligand Phe. Table 4 shows these interaction energies and the difference between naph-Ala and Phe.

It is seen that A314 main chain clashes with rotamer 1 of naph-Ala, while there is no main chain clash with rotamer2. Also E220 is one of the recognizing residue for the N terminal, and shows clash with rotamer 1. Hence only rotamer 2 was used for further consideration in following design steps.

TABLE 4A
Interaction energies (in kcal/mol) of each residue in the AARS
binding site with NBD-Ala (rotamer 1) and Phe.

| Residue | Naph-Ala (r1) | Phe | Difference |
|---|---|---|---|
| G284 | −1.70 | −0.51 | −1.19 |
| A283 | −2.79 | −1.72 | −1.08 |
| F260 | −4.45 | −3.52 | −0.93 |
| G282 | −2.78 | −1.92 | −0.86 |
| Q183 | −1.12 | −0.53 | −0.59 |
| G221 | −0.70 | −0.16 | −0.54 |
| S180 | −1.86 | −1.54 | −0.32 |
| L222 | −0.34 | −0.07 | −0.27 |
| M285 | −0.38 | −0.12 | −0.26 |
| F313 | −0.27 | −0.04 | −0.23 |
| F315 | −2.04 | −1.82 | −0.22 |
| V286 | −0.27 | −0.06 | −0.21 |
| G316 | −1.36 | −1.57 | 0.21 |
| V261 | 45.89 | −1.34 | 47.23* |
| E220 | 84.76 | −2.06 | 86.82* |
| A314 | 42990.79** | −1.25 | 42992.05** |

**main chain clash
The binding site is defined as with 6 Å of the side chain of NBD-Ala.
Residues labeled with * have at least 1 kcal/mol less favorable interactions with NBD-Ala compared to Phe.

TABLE 4B
Interaction energies (in kcal/mol) of each residue in the
AARS binding site with NBD-Ala (rotamer 2) and Phe.

| Residue | Naph-Ala (r2) | Phe | Difference |
|---|---|---|---|
| F260 | −5.06 | −3.52 | −1.54 |
| G282 | −3.02 | −1.92 | −1.10 |
| E262 | −1.40 | −0.41 | −0.99 |
| A283 | −2.54 | −1.72 | −0.82 |
| M285 | −0.65 | −0.12 | −0.53 |
| F315 | −2.34 | −1.82 | −0.51 |
| A265 | −0.57 | −0.17 | −0.40 |
| P263 | −0.46 | −0.08 | −0.38 |
| P259 | −0.45 | −0.16 | −0.30 |
| G264 | −0.17 | −0.01 | −0.16 |
| V286 | −0.22 | −0.06 | −0.15 |
| G284 | −0.37 | −0.51 | 0.14 |
| G316 | −1.21 | −1.57 | 0.36 |
| A314 | 2.79 | −1.25 | 4.04* |
| F258 | 17.69 | −3.99 | 21.68* |
| V261 | 2972007.99 | −1.34 | 2972009.33* |

From the difference of the interaction energy with rotamer 2 of naph-Ala and Phe, three residues (V261, F258, and A314) show at least 0.5 kcal/mol preference for binding Phe over naph-Ala. Therefore we choose to mutate these residues to change the binding preference of PheRS.

In the following step each of the 20 natural amino acids was tried at these three positions, one at a time. As described in the methods, a backbone dependent rotamer library was used to mutate a residue to another amino acid, and place the rotamer with the lowest energy with the rest of the protein. The mutated residue was minimized, and the interaction energies with naph-Ala, Phe and the rest of the protein were all calculated using Equation 1. Finally, a score for each mutation was calculated. Table 5 shows the results of the interaction energy of each mutation with naph-Ala and Phe, and the score of each mutation.

TABLE 5
Calculated interaction energies (in kcal/mol) of each mutation residue
with Phe and naph-Ala. A score is calculated as the difference of the
interaction energy between Phe and naph-Ala, plus 10% of the
clashing energy between the mutated residue and the rest of protein.

| V261 | Phe | naph-ala | score |
|---|---|---|---|
| G | −0.18 | −0.82 | −0.66 |
| A | −0.45 | 14.10 | 14.31 |
| S | −0.68 | 15.10 | 15.93 |
| C | −0.93 | 17.62 | 18.35 |
| N | −1.35 | 27.47 | 29.64 |
| D | −1.95 | 21.04 | 31.21 |
| Q | −1.31 | 30.20 | 32.69 |
| E | −2.03 | 24.30 | 35.13 |
| T | −1.10 | 34.69 | 35.92 |
| P | −1.30 | 35.25 | 39.64 |
| M | −1.47 | 39.83 | 42.08 |
| K | −0.92 | 36.77 | 43.36 |
| R | −0.94 | 44.29 | 50.94 |
| V | −1.17 | 68.41 | 69.55 |
| H | −1.70 | 74.77 | 79.36 |
| I | −1.57 | 197.60 | 205.87 |
| F | 34.38 | 329.71 | 302.19 |
| Y | 4.02 | 430.12 | 436.36 |
| L | 140.30 | 1074.13 | 957.24 |
| W | 303.54 | 1371.93 | 1136.81 |
| F258 | Phe | naph-Ala | score |
| R | 1261.58 | 1171.07 | −37.10 |
| G | −0.34 | −1.06 | −0.11 |
| A | −1.01 | 4.98 | 6.48 |
| S | −1.31 | 4.55 | 6.79 |
| C | −1.74 | 4.93 | 7.19 |
| M | −2.01 | 5.36 | 7.63 |
| T | −1.58 | 5.53 | 7.85 |
| N | 1.13 | 7.64 | 7.90 |
| I | 2.17 | 11.09 | 9.17 |
| V | −1.18 | 7.65 | 9.18 |
| W | −5.16 | 4.19 | 9.65 |
| H | −3.15 | 6.27 | 10.44 |
| Y | −3.84 | 7.04 | 11.30 |
| K | −1.53 | 5.03 | 11.34 |
| P | −1.42 | 11.17 | 12.99 |
| D | −0.34 | 5.33 | 14.13 |
| Q | −1.18 | 12.81 | 15.34 |
| E | 9.63 | 16.75 | 16.30 |
| F | 1.61 | 22.44 | 21.16 |
| L | 12.78 | 37.66 | 25.87 |
| A314 | Phe | naph-Ala | score |
| G | −0.81 | −0.53 | 0.16 |
| A | −1.21 | −0.10 | 0.82 |
| S | −1.62 | −0.56 | 1.21 |
| C | −1.40 | 3.51 | 4.78 |
| P | −1.57 | 3.03 | 5.28 |
| N | −1.36 | 2.89 | 5.28 |
| T | −1.49 | 3.95 | 5.48 |
| Q | −1.63 | 3.31 | 6.01 |
| W | −1.79 | 1.41 | 6.26 |
| I | 1.60 | 7.96 | 6.71 |
| V | 1.67 | 8.66 | 6.85 |
| L | −1.68 | 5.66 | 7.51 |
| H | −1.39 | 2.68 | 8.14 |
| D | −1.98 | −0.86 | 9.13 |
| M | −2.32 | 6.22 | 9.20 |
| F | −1.71 | 3.09 | 10.10 |
| K | −1.21 | 4.86 | 11.26 |
| E | −2.12 | 1.02 | 11.38 |
| R | −1.27 | 4.64 | 11.74 |
| Y | −1.65 | 2.52 | 12.75 |

Mutations with the lowest score were selected for generating mutants combinatorially. A negative score generally means that the mutation prefers the binding of naph-Ala over Phe, and the mutation does not cause a lot of clashing energies inside the protein. From Table 5, we chose G and A for V261; for F258, G, A, S, C, M, T, N, I, and V were selected; for A314, G, A, and S were chosen. Some of these mutations had a positive score, however, they were included because they are within 5 kcal/mol of the best mutation and the combined mutations sometimes have better selectivity for the non-natural amino acid. Also the selection depends on the total number of mutants being tractable.

In the next step, 2×9×3=54 mutants were generated using SCRWL. These mutants were first optimized by minimizing the mutated residues while the rest of protein was fixed. This was followed by minimizing the whole protein with AVGB implicit solvation. Finally the binding energies of the mutant with naph-Ala and its competitors from the natural amino acid pool (Phe, Tyr and Trp were considered here) were calculated using Equation 2. These results were listed in Table 6. The mutants were sorted by the differential binding energy between naph-Ala and the competitor with the best binding energy. The stability-check procedure was applied to determine if the mutant can fold correctly. This check was only done for those mutants showing at least 5 kcal/mol differential binding energy to naph-Ala.

TABLE 6
All PheRS mutants designed by COP for recognizing naph-Ala selectively.

| | | | naph- | | | | | |
|---|---|---|---|---|---|---|---|---|
| V261 | F258 | A314 | Ala | Trp | Phe | Tyr | Difference | Stability |
| G | G | G | 37.50 | 19.36 | 26.16 | 21.42 | 11.33 | N |
| G | N | G | 31.95 | 8.71 | 20.92 | 10.65 | 11.02 | N |
| G | V | G | 33.26 | 19.26 | 20.29 | 22.23 | 11.02 | N |
| G | A | G | 35.46 | 15.22 | 24.55 | 22.20 | 10.90 | N |
| G | G | A | 36.60 | 20.16 | 26.17 | 21.44 | 10.42 | N |
| G | M | G | 32.47 | 20.69 | 8.33 | 22.50 | 9.96 | N |
| G | A | A | 33.63 | 13.70 | 24.56 | 22.31 | 9.06 | N |
| G | S | G | 35.18 | 27.14 | 24.05 | 21.63 | 8.04 | N |
| G | V | A | 30.18 | 20.76 | 20.34 | 22.30 | 7.87 | N |
| G | N | A | 30.06 | 10.03 | 20.88 | 23.40 | 6.65 | N |
| G | I | G | 31.25 | 25.17 | 23.91 | 15.77 | 6.08 | Y |
| G | M | A | 28.40 | 22.35 | 8.38 | 22.41 | 5.98 | N |
| G | G | S | 32.20 | 20.28 | 26.22 | 14.78 | 5.97 | N |
| G | M | S | 22.57 | 16.85 | 7.54 | 14.91 | 5.72 | N |
| G | S | A | 33.31 | 28.12 | 24.00 | 21.71 | 5.19 | N |
| G | N | S | 24.52 | 8.16 | 20.02 | 17.11 | 4.49 | |
| A | V | G | 26.15 | 19.70 | 19.87 | 22.05 | 4.09 | |
| G | T | A | 33.01 | 29.19 | 15.97 | 20.08 | 3.82 | |
| A | A | G | 28.26 | 13.06 | 24.54 | 22.30 | 3.71 | |
| A | M | G | 25.66 | 13.63 | 8.12 | 22.45 | 3.20 | |
| A | A | A | 27.15 | 14.02 | 24.50 | 22.33 | 2.64 | |
| G | C | S | 26.72 | 18.48 | 24.18 | 14.82 | 2.53 | |
| G | C | G | 35.76 | 33.23 | 24.33 | 21.72 | 2.53 | |
| A | N | G | 22.81 | 4.66 | 20.91 | 11.71 | 1.89 | |
| A | S | G | 27.85 | 26.17 | 23.92 | 21.62 | 1.68 | |
| A | V | A | 23.75 | 20.94 | 19.81 | 22.18 | 1.56 | |
| G | S | S | 25.16 | 15.30 | 23.93 | 14.94 | 1.22 | |
| G | C | A | 33.84 | 32.76 | 24.42 | 21.89 | 1.08 | |
| A | N | A | 21.44 | 19.37 | 20.51 | 12.02 | 0.92 | |
| G | A | S | 25.36 | 19.19 | 24.57 | 15.59 | 0.78 | |
| A | M | S | 15.32 | 12.68 | 6.87 | 14.85 | 0.46 | |
| A | M | A | 22.66 | 14.70 | 8.12 | 22.37 | 0.28 | |
| G | I | A | 26.14 | 26.04 | 24.17 | 15.85 | 0.10 | |
| G | V | S | 21.65 | 21.55 | 19.74 | 15.50 | −0.10 | |
| A | S | A | 26.79 | 26.98 | 23.98 | 21.69 | −0.19 | |
| G | T | G | 34.95 | 36.02 | 15.87 | 19.95 | −1.07 | |
| A | C | G | 28.59 | 30.45 | 24.27 | 21.79 | −1.86 | |
| A | C | S | 21.73 | 18.26 | 24.22 | 14.80 | −2.50 | |
| A | S | S | 21.30 | 14.09 | 23.82 | 14.87 | −2.53 | |
| A | A | S | 21.64 | 18.47 | 24.51 | 15.59 | −2.88 | |
| A | G | S | 23.27 | 23.12 | 26.18 | 14.76 | −2.92 | |
| A | T | G | 27.79 | 30.84 | 15.97 | 19.91 | −3.05 | |
| A | G | G | 22.80 | 16.37 | 26.35 | 21.52 | −3.56 | |
| A | C | A | 27.21 | 31.31 | 24.26 | 21.81 | −4.10 | |
| A | G | A | 22.05 | 16.86 | 26.25 | 21.60 | −4.21 | |
| G | T | S | 25.85 | 30.24 | 15.71 | 13.03 | −4.39 | |
| A | V | S | 17.10 | 22.72 | 19.50 | 15.35 | −5.62 | |
| A | N | S | 20.06 | 17.50 | 26.32 | 17.02 | −6.27 | |
| A | T | A | 26.47 | 34.42 | 15.81 | 20.10 | −7.95 | |
| A | T | S | 21.15 | 31.98 | 15.78 | 13.08 | −10.83 | |
| A | I | G | 8.75 | 7.94 | 27.25 | 15.93 | −18.51 | |
| G | I | S | 6.32 | 7.54 | 24.96 | 9.34 | −18.65 | |
| A | I | A | 3.76 | 17.72 | 26.06 | 15.89 | −22.31 | |
| A | I | S | 5.39 | 6.50 | 27.44 | 9.24 | −32.84 | |

Among the 54 mutants designed by COP, 15 of them showed good binding and selectivity toward naph-Ala. However, only one mutant passed the stability check. Thus COP designed only one mutant in this case, and it is V261G-F258I-A314G.

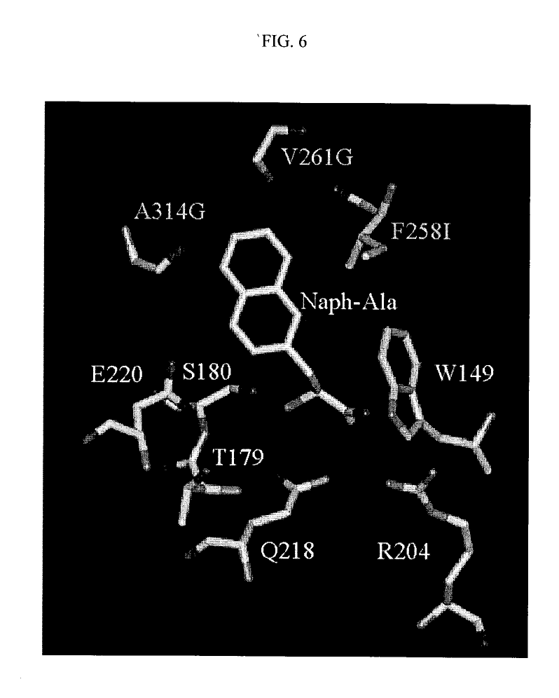

Previously a V261G-A314G PheRS mutant has been designed computationally and tested experimentally to recognize p-keto-Phe selectively. Naph-Ala is a little bulkier than p-keto-Phe, therefore it requires one more mutation (F258I) for PheRS to bind it. FIG. 6 shows the binding site of the designed mutant PheRS along with naph-Ala. The three mutations open the space for the extra six-member ring in the naphthyl group. The recognizing forces for the zwitterion part of the ligand remain the same as in wild-type PheRS. The NH₃⁺ group interacts with E220, T179, S180 and Q218, while the COO⁻ forms several hydrogen bonds with W149, R204 and Q218.

In summary, we have used our protein design tool COP to redesign a mutant PheRS for recognizing naph-Ala from 54 mutants we tried. This V261G-F258I-A314G mutant shows both a good binding energy (31.25 kcal/mol) and a differential binding energy (6.08 kcal/mol) to naph-Ala. We predict that the COP designed mutant should allow sufficient incorporation of naph-Ala in vivo.

### REFERENCES

1. Petka, W. A., et al., Reversible hydrogels from self-assembling artificial proteins. Science, 1998.281 (5375)”p. 389-92.
2. Tang, Y., Ghirlanda, G., Petka, W. A., Nakajima, T., DeGrado, W. F. & Tirrell, D. A., Fluorinated coiled-coil proteins prepared in vivo display enhanced thermal and chemical stability. Angewandte Chemie-International Edition, 2001. 40(8): p. 1494-1498.
3. Noren, C. J., et al., A general method for site specific incorporation of unnatural amino acids into proteins. Science, 1989. 244 (4901): p. 182-8.
4. Henrickson, W. A., J. R. Horton, and D. M. LeMaster, Selenomethionyl proteins produced for analysis by multiwavelength anomalous diffraction (MAD): a vehicle for direct determination of three-dimensional structure. EMBO J, 1990. 9(5): p. 1665-72.
5. Richmond, M. H., Random Replacement of PhenylAlanine by p-Fluorophenylanine in alkaline Phosphatase(s) Formed during Biosynthesis by E. coli. J. Mol. Biol., 1963. 6 p. 284-294.
6. Yoshikawa, E. Fournier, M. J., Mason, T. L., & Tirrell, D. A., Genetically Engineered Fluoropolymers. Synthesis of Repetitive Polypeptides Contained p-FiuorophenylAlanine Residues. Macromolecules, 1994. 27: p. 5471-5475.
7. van Hest, J. C. M., Kuck, K. L., & Tirrell, D. A., Efficient Incorporation of Unsaturated Methionine Analogues into Proteins in vivo, J. Am. Chem. Soc., 2000. 122(7): p. 1282-1288.
8. Kothakota, S., Mason, T. L., Tirrell, D. A., & Fournier, M. J., Biosynthesis of a Periodic Protein Containing 3-Thienylanine: A Step Toward Genetically Engineered Conducting Polymers. J. Am. Chem. Soc., 1995. 117: p. 536-537.
9. Cowie, D. B., & Cohen, G. N., Biosynthesis by Escherichia coli of Active altered Proteins Containing Selenium Instead of Sulfur. Biochim, Biophys. Acta, 1957.26: p. 252-261.
10. Budisa, N., Steipe, B., Demange, P. Eckerskorn, C., Kellermann, J., & Huber, R., High-Level Biosynthetic Substitution of Methionine in Proteins by its Analogs 2—Aminohexanoic Acid, Selenomethionine, Telluromethionine, and Ethionine in E. coli. Eur. J. Biochem., 1995, 230: p. 788-796.
11. Duewel, H., Daub, E., Robinson, V., & Honek, J. F., Incorporation of Trifuluormethionine into a Phage Lysozyme: Implications and a New Marker for Use in Protein 19F NMR. Biochemistry, 1997. 36: p. 3404-3416s.
12. Ibba, M., & Hennecke, H., FEBS Lett., 1995. 364: p. 272-275.
13. Ibba, M., & Hennecke, H., FEBS Lett., 1995. 364: p. 272-275.
14. Liu, D. R., et al., Engineering a tRNA and aminoacyl-tRNA synthetase for the site-specific incorporation of unnatural amino acids into proteins in vivo. Proc Natl Acad Sci USA, 1997. 94(19): p. 10092-7.
15. Kowal, A. K., C. Kohrer, and U. L. RajBhandary, Twenty-first aminoacyl-tRNA synthetase-suppressor tRNA pairs for possible use in site-specific incorporation of amino acid analogues into proteins in eukaryotes and in eubacteria. Proc Natl Acad Sci USA. 2001. 98(5): p. 2268-73.
16. Wang, L., et al., Expanding the genetic code of Escherichia coli. Science, 2001. 292(5516): P. 498-500.
17. Bower, M. J., F. E. Cohen, and R. L. Dunbrack, Jr., Prediction of protein side-chain rotamers from a backbone-dependent rotamer library: a new homology modeling tool. J. Mol. Biol, 1997. 267(5): p. 1268-82.

The practice of the present invention will employ, unless otherwise indicated, conventional techniques of molecular biology, cell biology, cell culture, microbiology and recombinant DNA, which are within the skill of the art. Such techniques are explained fully in the literature. See, for example, Molecular Cloning: A Laboratory Manual, 2^{nd} Ed., ed. By Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al.; U.S. Pat. No. 4,683,195; Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987).

The contents of all cited references (including literature references, issued patents, published patent applications as cited throughout this application) are hereby expressly incorporated by reference.

Equivalents

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

What is claimed is:

1. A method for designing a mutant of a polypeptide, said mutant preferentially binds an analog ligand (AL) over at least one inactive ligand (IL) of said polypeptide, comprising:
(a) providing (i) an atomic coordinate model for said polypeptide, which model includes coordinates for at least the binding pocket residues for said IL; (ii) an IL rotamer of the most favorable solution conformation, and (iii) one or more AL rotamers, each more stable in solution than a pre-determined level of solution conformation energy;
(b) docking said IL rotamer and each said AL rotamers into said binding pocket, wherein the backbone of said IL rotamer and said AL rotamers remain unchanged with respect to one another;
(c) for each said AL rotamers docked into said binding pocket, identifying, in said binding pocket, varying residues which have less favorable interactions with said AL rotamer than with said IL rotamer;
(d) identifying a subset of mutations for each of said varying residues identified in (c), wherein each of said mutations alone allows said polypeptide with fixed backbone to form a more favorable interaction with said AL rotamer than with said IL rotamer;
(e) for each said AL rotamer, generating candidate combinatorial mutations including mutations in at least two of said varying residues, and selecting combinatorial mutations which form a more favorable interaction with said AL rotamer than with said IL rotamer;
(f) for each said AL rotamer, selecting stable combinatorial mutations by optimizing each combinatorial mutations selected in (e) with no ligand in said binding pocket.

2. The method of claim 1, further comprising:
(g) for each stable combinatorial mutation selected in (f), identifying one or more specific combinatorial mutations that preferentially bind said AL rotamer over a pool of ILs structurally similar to said IL.

3. The method of claim 1 or 2, further comprising:
(h) generating each combinatorial mutation finally selected in (f) or (g) and testing in vivo and/or in vitro for selectively incorporating said AL into proteins over said IL, or said pools of ILs structurally similar to said IL.

4. The method of claim 1, wherein said polypeptide is an Aminoacyl tRNA Synthetase (AARS), said IL is a natural ligand of said AARS, and said AL is an analog of said natural ligand.

5. The method of claim 4, wherein said natural ligand is one of the twenty amino acids usually found in natural proteins.

6. The method of claim 4, wherein said AARS is a phenoAlanyl-tRNA Synthetase (PheRS) or a tyrosyl-tRNA Synthetase (TyrRS).

7. The method of claim 4, wherein said AARS is from a bacteria.

8. The method of claim 4, wherein said AARS is from a eukaryote.

9. The method of claim 4, wherein said AL is a derivative of at least one of the 20 natural amino acids, with one or more functional groups not present in natural amino acids.

10. The method of claim 9, wherein said functional group is selected from the group consisting of: bromo-, iodo-, ethynyl-, cyano-, azido-, aceytyl, aryl ketone, a photolabile group, a fluorescent group, and a heavy metal.

11. The method of claim 9, wherein said AL is a derivative of Phe or Tyr.

12. The method of claim 1, wherein said polypeptide is a G-Protein Coupled Receptor (GPCR) selected from: an adrenergic receptor (AR), an endothelial differential gene (EDG), an Olfactory Receptor (OR), or a Sweet Receptor (SR).

13. The method of claim 1, wherein said binding pocket residues comprise residues having at least one atom within a pre-defined distance from any atom of said AL or said IL.

14. The method of claim 13, wherein said pre-defined distance is 6 \acute{Å}.

15. The method of claim 1, wherein said AL rotamers are provided in (iii) by generating various candidate rotamers of said AL over a grid of dihedral angles, and calculating stability of all candidate rotamers.

16. The method of claim 15, wherein the stability of said one or more AL rotamers are calculated using quantum mechanics, or a suitable force field with molecular mechanics, or both.

17. The method of claim 1, wherein said IL rotamer or said AL rotamers is/are docked into said binding pocket based on a known three-dimensional complex structure of said IL and said polypeptide.

18. The method of claim 1 or 4, wherein said IL rotamer or said AL rotamers is/are docked into said binding pocket based on a docking algorithm.

19. The method of claim 18, wherein said docking algorithm is HIERDOCK.

20. The method of claim 1, wherein step (c) is effectuated by calculating and comparing non-bond energy contribution towards said AL rotamer and said IL rotamer for each binding pocket residues.

21. The method of claim 20, wherein said non-bond energy contribution is calculated using a force field.

22. The method of claim 21, wherein said force field is selected from: AMBER, AMBER94, AMBER/OPLS, OPLS, OPLS-AA, CHARMM, CHARMM22, Discover, ECEPP/2, GROMOS, MM2, MM3, MM4, MMFF, MMFFs, MMFF94, or UFF.

23. The method of claim 21, wherein said force field is a DREIDING force field.

24. The method of claim 23, wherein said DREIDING force field considers function forms for Coulomb, van der Waals, and hydrogen bond interactions.

25. The method of claim 24, wherein the dielectric constant for said Coulomb functional form is distance dependent or distance independent.

26. The method of claim 24, wherein the charge for either said binding pocket residues, or said AL/IL rotamer, or both can be varied in said Coulomb functional form.

27. The method of claim 26, wherein said charge includes charge from experiment, or charge based on a model selected from: QEq, Del Re, MPEOE, or Gasteiger/PEOE.

28. The method of claim 24, wherein the functional form of said van der Waals interaction uses a Leonard-Jones potential.

29. The method of claim 28, wherein said Leonard-Jones potential is a 6-12 or 6-10 Leonard-Jones potential.

30. The method of claim 24, wherein the functional form of said van der Waals interaction uses a Morse potential.

31. The method of claim 24, wherein the functional form of said hydrogen bond interaction uses a three-body form.

32. The method of claim 24, wherein the functional form of said hydrogen bond interaction uses a two-body form or a four-body form.

33. The method of claim 20, wherein said non-bond energy contribution is calculated using quantum mechanics (QM).

34. The method of claim 20, wherein in step (c), said AL rotamer docked into said binding pocket has less than a threshold level of clash with the backbone of said polypeptide.

35. The method of claim 34, wherein said threshold is 50%.

36. The method of claim 34, wherein said varying residues are clash residues.

37. The method of claim 36, wherein for each said varying residue, step (d) is effectuated by substituting the wild-type amino acid at said varying residue with all 19 natural amino acids, one at a time, and selecting all substitutions that favor binding to said AL rotamer over said IL rotamer.

38. The method of claim 37, wherein the side-chain conformation of each of the 19 substituted natural amino acids is generated over a grid of dihedral angles.

39. The method of claim 37, wherein the side-chain conformation of each of the 19 substituted natural amino acids is generated from a backbone-dependent rotamer library.

40. The method of claim 39, wherein the wild-type amino acid at said varying residue is substituted with all 19 natural amino acids, one at a time, using SCWRL.

41. The method of claim 37, wherein the wild-type amino acid at said varying residue is substituted with all 19 natural amino acids, one at a time, using SCAP.

42. The method of claim 37, wherein the wild-type amino acid at said varying residue is substituted with all 19 natural amino acids, one at a time, using a side-chain modeling method based on branch-and-bound or dead-end-elimination algorithm.

43. The method of claim 37, further comprising optimizing the side-chain of each of the 19 substituted natural amino acids after substitution but before selection.

44. The method of claim 43, wherein said optimization is carried out by energy minimization.

45. The method of claim 44, wherein said energy minimization utilizes a force field.

46. The method of claim 45, wherein said force field is a DREIDING force field.

47. The method of claim 43, wherein said optimization is carried out by Molecular Dynamics or by using Monte Carlo techniques.

48. The method of claim 37, wherein substitutions that favor binding to said AL rotamer are selected based on a score for each substitution, said score comprising a weighed sum of (I) the differential non-bond interaction energy of the substituted varying residue with said IL rotamer and said AL rotamer, and (II) the differential non-bond interaction energy of the substituted varying residue with the remaining residues of said polypeptide.

49. The method of claim 48, wherein said differential non-bond interaction energy of (I) and said differential non-bond interaction energy of (II) are independently calculated using a force field or quantum mechanics.

50. The method of claim 48, wherein said weighed sum comprises 75-100% of said differential non-bond interaction energy of (I).

51. The method of claim 48, wherein said score includes desolvation penalty for said AL rotamer and said IL rotamer.

52. The method of claim 51, wherein said desolvation penalty is calculated using a continuum implicit solvent model.

53. The method of claim 52, wherein said continuum implicit solvent model is a Surface Generalized Born (SGB) model, a Solvent Accessible Surface Area (SASA)/Analytical Volume Generalized Born (AVGB) model, or a Poisson-Boltzmann (PB) model.

54. The method of claim 53, wherein said desolvation penalty calculation further includes adding explicit solvent molecules.

55. The method of claim 34, wherein said varying residues are opportunity residues.

56. The method of claim 55, wherein said opportunity residues are identified after identification of clash residues.

57. The method of claim 56, wherein said opportunity residues stabilizes said AL rotamer or destabilizes said IL rotamer or both.

58. The method of claim 57, wherein said opportunity residues takes advantage of hydrogen bond donor or acceptor atoms that are different between said AL rotamer and said IL rotamer.

59. The method of claim 57, wherein said opportunity residues are identified by their proximity to a large void space in said binding pocket after identifying and mutating clash residues.

60. The method of claim 56, wherein for each said varying residue, step (d) is effectuated by substituting the wild-type amino acid at said varying residue with all 19 natural amino acids, one at a time, and selecting all substitutions that favor binding to said AL rotamer over said IL rotamer.

61. The method of claim 1, wherein in step (e), side-chains of each generated combinatorial mutation are optimized to generate optimal side-chain conformation for each combinatorial mutation.

62. The method of claim 61, wherein said optimization is carried out using a DREIDING force field with conjugate gradient minimization.

63. The method of claim 61, wherein after side-chain optimization, each of said combinatorial mutation is globally optimized both with said AL rotamer and with said IL rotamer.

64. The method of claim 63, wherein said global optimization both with said AL rotamer and with said IL rotamer is independently carried out using a force field, Molecular Dynamics, or Monte Carlo techniques.

65. The method of claim 63, wherein the backbone of each said combinatorial mutation is not fixed during global optimization.

66. The method of claim 1, wherein in step (e), combinatorial mutations which form a more favorable interaction with said AL rotamer than with said IL rotamer are selected based on the differential binding energy of said combinatorial mutations with said AL rotamer and said IL rotamer.

67. The method of claim 66, wherein said differential binding energy is calculated with a force field or quantum mechanics.

68. The method of claim 67, wherein said differential binding energy is calculated with a DREIDING force field with SGB salvation or AVGB salvation.

69. The method of claim 68, wherein said differential binding energy is calculated with Equation 2.

70. The method of claim 1, wherein the binding energies for said interactions in steps (c)-(e) are calculated using Potential of Mean Force (PMF), average dynamic free energy, Free Energy Perturbation (FEP), or methods based on thermodynamic cycles.

71. The method of claim 1, wherein in step (f), for each combinatorial mutations selected in (e), the varying residue side-chains are reselected from a backbone-dependent rotamer library, and each said combinatorial mutation is globally optimized with no ligand, using a force field or quantum mechanics with a continuum implicit solvent model.

72. The method of claim 71, wherein said global optimization include explicit water in said binding pocket.

73. The method of claim 71, further including calculating the differential binding energy for the globally optimized combinatorial mutation with said AL rotamer and said IL rotamer, using a force field or quantum mechanics with a continuum implicit solvent model.

74. The method of claim 73, wherein the global optimization and the differential binding energy are calculated using Equation 2.

75. A peptide or polypeptide incorporating one or more amino acid analogs, which peptide or polypeptide was produced using a mutant AARS designed by the method of claim 4.

76. A method for conducting a biotechnology business comprising:
(i) identifying one or more mutant forms of an AARS sequence, by the method of claim 4, said mutant preferentially binds to an amino acid analog of a natural amino acid substrate of said AARS;
(ii) providing a translation system including:
(a) a transcript, or means for generating a transcript that encodes a peptide or polypeptide,
(b) an mutant AARS having said identified mutant AARS sequence(s), and
(c) said amino acid analog,
under circumstances wherein said mutant AARS catalyzes incorporation of said amino acid analog in said peptide or polypeptide.

77. The business method of claim 76, further comprising the step of providing a packaged pharmaceutical including the peptide or polypeptide, and instructions and/or a label describing how to administer or use said peptide or polypeptide.

78. A recombinant AARS protein generated by the method of claim 4, said AARS protein comprising an optimized protein sequence that incorporates an amino acid analog of a natural amino acid substrate of said AARS into a protein in vivo.

79. A nucleic acid sequence encoding a recombinant AARS protein according to claim 78.

80. An expression vector comprising the nucleic acid sequence of claim 79.

81. A host cell comprising the nucleic acid sequence of claim 79.

82. An apparatus for designing a mutant of a polypeptide, said mutant preferentially binds an analog ligand (AL) over at least one inactive ligand (IL) of said polypeptide, said apparatus comprising:
(A) means for providing (i) an atomic coordinate model for said polypeptide, which model includes coordinates for at least the binding pocket residues for said IL; (ii) an IL rotamer of the most favorable solution conformation, and (iii) one or more AL rotamers, each more stable in solution than a pre-determined level of solution conformation energy;
(B) means for docking said IL rotamer and each said AL rotamers into said binding pocket, wherein the backbone of said IL rotamer and said AL rotamers remain unchanged with respect to one another;
(C) for each said AL rotamers docked into said binding pocket, means for identifying, in said binding pocket, varying residues which have less favorable interactions with said AL rotamer than with said IL rotamer;
(D) means for identifying a subset of mutations for each of said varying residues identified in (C), wherein each of said mutations alone allows said polypeptide with fixed backbone to form a more favorable interaction with said AL rotamer than with said IL rotamer;
(E) for each said AL rotamer, means for generating candidate combinatorial mutations including mutations in at least two of said varying residues, and selecting combinatorial mutations which form a more favorable interaction with said AL rotamer than with said IL rotamer; and,
(F) for each said AL rotamer, means for selecting stable combinatorial mutations by optimizing each combinatorial mutations selected in (E) with no ligand in said binding pocket.

83. The method of claim 82, wherein said polypeptide is an Aminoacyl tRNA Synthetase (AARS), said IL is a natural ligand of said AARS, and said AL is an analog of said natural ligand.

84. A computer system for designing a mutant of a polypeptide, said mutant preferentially binds an analog ligand (AL) over at least one inactive ligand (IL) of said polypeptide, said computer system comprising computer instructions for:
(a) providing (i) an atomic coordinate model for said polypeptide, which model includes coordinates for at least the binding pocket residues for said IL; (ii) an IL rotamer of the most favorable solution conformation, and (iii) one or more AL rotamers, each more stable in solution than a pre-determined level of solution conformation energy;
(b) docking said IL rotamer and each said AL rotamers into said binding pocket, wherein the backbone of said IL rotamer and said AL rotamers remain unchanged with respect to one another;
(c) for each said AL rotamers docked into said binding pocket, identifying, in said binding pocket, varying residues which have less favorable interactions with said AL rotamer than with said IL rotamer;
(d) identifying a subset of mutations for each of said varying residues identified in (c), wherein each of said mutations alone allows said polypeptide with fixed backbone to form a more favorable interaction with said AL rotamer than with said IL rotamer;
(e) for each said AL rotamer, generating candidate combinatorial mutations including mutations in at least two of said varying residues, and selecting combinatorial mutations which form a more favorable interaction with said AL rotamer than with said IL rotamer;
(f) for each said AL rotamer, selecting stable combinatorial mutations by optimizing each combinatorial mutations selected in (e) with no ligand in said binding pocket.

85. The method of claim 84, wherein said polypeptide is an Aminoacyl tRNA Synthetase (AARS), said IL is a natural ligand of said AARS, and said AL is an analog of said natural ligand.

86. A computer-readable medium storing a computer program executable by a plurality of server computers, the computer program comprising computer instructions for:
(a) providing (i) an atomic coordinate model for said polypeptide, which model includes coordinates for at least the binding pocket residues for said IL; (ii) an IL rotamer of the most favorable solution conformation, and (iii) one or more AL rotamers, each more stable in solution than a pre-determined level of solution conformation energy;
(b) docking said IL rotamer and each said AL rotamers into said binding pocket, wherein the backbone of said IL rotamer and said AL rotamers remain unchanged with respect to one another;
(c) for each said AL rotamers docked into said binding pocket, identifying, in said binding pocket, varying residues which have less favorable interactions with said AL rotamer than with said IL rotamer;
(d) identifying a subset of mutations for each of said varying residues identified in (c), wherein each of said mutations alone allows said polypeptide with fixed backbone to form a more favorable interaction with said AL rotamer than with said IL rotamer;
(e) for each said AL rotamer, generating candidate combinatorial mutations including mutations in at least two of said varying residues, and selecting combinatorial mutations which form a more favorable interaction with said AL rotamer than with said IL rotamer;
(f) for each said AL rotamer, selecting stable combinatorial mutations by optimizing each combinatorial mutations selected in (e) with no ligand in said binding pocket.

87. The method of claim 86, wherein said polypeptide is an Aminoacyl tRNA Synthetase (AARS), said IL is a natural ligand of said AARS, and said AL is an analog of said natural ligand.

88. A computer data signal embodied in a carrier wave, comprising computer instructions for:
(a) providing (i) an atomic coordinate model for said polypeptide, which model includes coordinates for at least the binding pocket residues for said IL; (ii) an IL rotamer of the most favorable solution conformation, and (iii) one or more AL rotamers, each more stable in solution than a pre-determined level of solution conformation energy;
(b) docking said IL rotamer and each said AL rotamers into said binding pocket, wherein the backbone of said IL rotamer and said AL rotamers remain unchanged with respect to one another;
(c) for each said AL rotamers docked into said binding pocket, identifying, in said binding pocket, varying residues which have less favorable interactions with said AL rotamer than with said IL rotamer;
(d) identifying a subset of mutations for each of said varying residues identified in (c), wherein each of said mutations alone-allows said polypeptide with fixed backbone to form a more favorable interaction with said AL rotamer than with said IL rotamer;
(e) for each said AL rotamer, generating candidate combinatorial mutations including mutations in at least two of said varying residues, and selecting combinatorial mutations which form a more favorable interaction with said AL rotamer than with said IL rotamer;
(f) for each said AL rotamer, selecting stable combinatorial mutations by optimizing each combinatorial mutations selected in (e) with no ligand in said binding pocket.

89. The method of claim 88, wherein said polypeptide is an Aminoacyl tRNA Synthetase (AARS), said IL is a natural ligand of said AARS, and said AL is an analog of said natural ligand.

90. An apparatus comprising a computer readable storage medium having instructions stored thereon for:
(i) accessing a datafile representative of the coordinates for a plurality of different rotamers of one or more amino acids and one or more analogs of said amino acids;
(ii) accessing a datafile representative of a set of structure coordinates for amino acid residues that define a binding pocket for an aminoacyl tRNA synthetase;
(iii) a set of modeling routines for
(a) calculating differential non-bond interaction energies between residues of the binding pocket with both the amino acids and the amino acid analogs;
(b) altering one or more amino acid residues in the binding pocket for the aminoacyl tRNA synthetase (AARS) to produce one or more sets of altered AARS structure coordinates defining altered binding pockets that differentially favor binding of the amino avid analogs over the amino acids;
(c) generating a list representative of optimized AARS sequences having preferential interactions with said amino acid analogs over said amino acids.

91. A method for synthesizing a peptide or protein incorporating one or more amino acid analogs, comprising providing a translational system including:
(i) a transcript, or means for generating a transcript, that encodes a peptide or polypeptide, and
(ii) one or more mutant AARS having a mutated binding pocket, each said mutant AARS catalyze preferential incorporation of an amino acid analog of the natural amino acid substrate of said AARS into said peptide or protein under the conditions of the translation system.

92. The method of claim 91, wherein said translation system is a whole cell that expresses said one or more AARS.

93. The method of claim 91, wherein said translation system is a cell lysate or reconstituted protein preparation that is translation competent.

94. The method of claim 91, wherein at least one of said mutant AARS catalyzes incorporation of said amino acid analog with a Kcat at least 10 fold greater than the wild-type AARS.

95. The method of claim 91, wherein at least one of said mutant AARS catalyzes incorporation of said amino acid analog with a K_{cat} at least 10 fold greater than the K_{cat} of the incorporation of natural amino acid substrate of the wild-type AARS.

96. The method of claim 91, wherein at least one of said mutant AARS catalyzes incorporation of said amino acid analog with a K_{cat} at least 2 fold greater than the K_{cat} of the incorporation of natural amino acid substrate of the wild-type AARS.

97. The method of claim 91, wherein at least one of said mutant AARS has a sequence identified by the method of claim 4.
